SEQUENCE LISTING (6 sequences)

SEQ ID NO: 1 - DNA, 20 bp, Artificial Sequence
  Fw primer chrX
attttgatcggcttagacca

SEQ ID NO: 2 - DNA, 18 bp, Artificial Sequence
  Rev primer chrX
ggttgatcaaagccagtc

SEQ ID NO: 3 - DNA, 20 bp, Artificial Sequence
  Fw primer nested PCR
gtccagctttgtcctgtatt

SEQ ID NO: 4 - DNA, 20 bp, Artificial Sequence
  Rev primer nested PCR
gtcatggctggtcaagatag

SEQ ID NO: 5 - DNA, 54 bp, Artificial Sequence
  Fwd primer nested PCR including Illumina sequencing adapters and an index sequence
gtgactggagttcagacgtgtgctcttccgatctgtccagctttgtcctgtatt

SEQ ID NO: 6 - DNA, 53 bp, Artificial Sequence
  Rev primer nested PCR including Illumina sequencing adapters and an index sequence
acactctttccctacacgacgctcttccgatctgtcatggctggtcaagatag

(19) United States
(12) Patent Application Publication
(10) Pub. No.: US 2023/0170042 A1
(43) Pub. Date: Jun. 1, 2023

(54) STRUCTURAL VARIATION DETECTION IN CHROMOSOMAL PROXIMITY EXPERIMENTS

(71) Applicant: KONINKLIJKE NEDERLANDSE AKADEMIE VAN WETENSCHAPPEN, Amsterdam (NL)

(72) Inventors: Wouter Leonard De Laat, Utrecht (NL); Amin Allahyar, Utrecht (NL); Erik Cornelis Splinter, Utrecht (NL)

(21) Appl. No.: 17/919,970

(22) PCT Filed: Apr. 23, 2021
(86) PCT No.: PCT/NL2021/050268

§ 371 Date: Oct. 19, 2022

(30) Foreign Application Priority Data
Apr. 23, 2020 (EP) 20171092.8
Nov. 2, 2020 (EP) 20205208.0

(51) Int. Cl. G16B 20/00; C12Q 1/6827

(52) U.S. Cl. CPC G16B 20/00; C12Q 1/6827

(57) ABSTRACT

The present invention relates to the field of molecular biology and more in particular to DNA technology. The invention relates to strategies for assessing structural integrity of DNA sequences of a genomic region of interest, which has clinical applications in diagnostics and personalized cancer therapy. In particular, the invention provides a method of detecting a chromosomal rearrangement involving a genomic region of interest.

### CROSS REFERENCE TO RELATED APPLICATIONS

This application is a national phase entry under 35 U.S.C. § 371 of International Patent Application PCT/NL2021/050268, filed Apr. 23, 2021, designating the United States of America and published in English as International Patent Publication WO 2021/215927 on Oct. 28, 2021, which claims the benefit under Article 8 of the Patent Cooperation Treaty to European Patent Application Serial No. 20171092.8, filed Apr. 23, 2020, and which claims the benefit to European Patent Application Serial No. 20205208.0, filed Nov. 2, 2020 the entireties of which are hereby incorporated by reference.

### FIELD OF THE INVENTION

The present invention relates to the field of molecular biology and more in particular to DNA technology. The invention relates to strategies for assessing structural integrity of DNA sequences of a genomic region of interest, which has clinical applications in diagnostics and personalized cancer therapy.

In particular a method of detecting a chromosomal rearrangement for DNA reads and a genomic region of interest is provided. An observed proximity score is assigned (101) to genomic fragments. An expected proximity score is assigned (102) to each of at least one genomic fragment of the plurality of genomic fragments, based on the observed proximity scores of the plurality of genomic fragments, wherein the expected proximity score is an expected value of the proximity score of the at least one of the plurality of genomic fragments. An indication is generated (104) of a likelihood that said at least one genomic fragment of the plurality of genomic fragments is involved in a chromosomal rearrangement, based on the observed proximity score of said at least one genomic fragment of the plurality of genomic fragments and the expected proximity score of said at least one genomic fragment of the plurality of genomic fragments.

### BACKGROUND

There are a series of techniques (3C, 4C, 5C, Hi-C, ChIA-PET, HiChIP, Targeted Locus Amplification (TLA), capture-C, promoter-capture HiC, to name a few (see Denker & de Laat, Genes & Development 2016) that are based on proximity-ligation in 3D space of the nucleus: the fragmentation and subsequent re-ligation of DNA inside the cell nucleus (in situ). In most proximity-ligation assays, prior to fragmentation chromatin is first crosslinked to help preserving the original 3D conformation, but there are also crosslinking-free in situ fragmentation and proximity ligation technologies (e.g. Brant et al., Mol Sys Biol 2016). These procedures give ligation products between spatially proximal (i.e. interacting) DNA fragments and as such can be used to analyze chromosome folding inside the cell nucleus. In addition to proximity ligation methods there are other nuclear proximity methods such as SPRITE (split-pool recognition of interactions by tag extension) (Quinodoz et at, Cell 2018) that depend on crosslinking but not on ligation to identify nuclear proximal DNA sequences. However, the dominant signal contributing to proximity in the nuclear (cellular) space is linear proximity. Linearly adjacent DNA fragments on a chromosome will inevitably be physically proximal which in turn increases their likelihood to be found together in proximity-ligated products or other nuclear proximity assays. In general, this propensity decays exponentially with increased linear distance between pairs of fragments on the chromosome.

This feature enables nuclear proximity methods, including the proximity ligation assays to sensitively detect chromosomal rearrangements that cause changes in the linear structure of the chromosomes. For example, performing such a proximity ligation assay and analyzing ligation products formed with DNA fragments near a translocation site (close to where two different chromosomes are fused) would give very frequent ligation products between the two fused partners.

De Laat and Grosveld disclosed in WO2008084405 that rearrangements can be detected based on (a) ‘the difference in interaction frequency between the DNA sequences of diseased cells and non-diseased cells’ and/or (b) ‘a transition from low to high interaction frequencies’.

### SUMMARY OF THE INVENTION

In one aspect, the disclosure provides a method for confirming the presence of a chromosomal breakpoint junction, fusing a candidate rearrangement partner to a position within a genomic region of interest, said method comprising:

a. performing a proximity assay on a DNA comprising sample to generate a plurality of proximity linked products;
b. enriching for proximity linked products that comprise genomic fragments comprising sequences flanking the 5′ end of the genomic region of interest,
wherein said proximity linked products further comprise genomic fragments being in proximity to said genomic fragments comprising sequences flanking the 5′ end of the genomic region of interest; sequencing said proximity linked products to produce sequencing reads,
mapping to a reference sequence the sequences of the genomic fragments that are in proximity to said genomic fragments comprising sequences flanking the 5′ end of the genomic region of interest;
c. enriching for proximity linked products that comprise genomic fragments comprising sequences flanking the 3′ end of the genomic region of interest,
wherein said proximity linked products further comprise genomic fragments being in proximity to said genomic fragments comprising sequences flanking the 3′ end of the genomic region of interest; sequencing said proximity linked products to produce sequencing reads,
mapping to a reference sequence the sequences of the genomic fragments that are in proximity to said genomic fragments comprising sequences flanking the 3′ end of the genomic region of interest;
d. identifying, as a candidate rearrangement partner, at least one genomic fragment based on the proximity frequency of said genomic fragment with the genomic region of interest or genomic fragments comprising sequences flanking the genomic region of interest,
e. determining whether genomic fragments of the candidate rearrangement partner that are in proximity to said genomic fragments comprising sequences flanking the 5′ end of the genomic region of interest and genomic fragments of the candidate rearrangement partner that are in proximity to said genomic fragments comprising sequences flanking the 3′ end of the genomic region of interest are overlapping or linearly separated,
wherein linear separation of said candidate rearrangement partner genomic fragments is indicative of a chromosomal breakpoint junction within the genomic region of interest.
Preferably, the proximity assay is a proximity ligation assay that generates a plurality of proximity ligated products.
Preferably, step d) comprises assigning (101) an observed proximity score to each of a plurality of genomic fragments of a genome, the observed proximity score of each genomic fragment being indicative of a presence in the dataset of at least one sequencing read in proximity to the genomic region of interest and comprising a sequence corresponding to the genomic fragment;
assigning (102) an expected proximity score to each of at least one genomic fragment of the plurality of genomic fragments, based on the observed proximity scores of the plurality of genomic fragments, wherein the expected proximity score comprises an expected value of the proximity score of the at least one of the plurality of genomic fragments; and generating (103) an indication of a likelihood that said at least one genomic fragment of the plurality of genomic fragments is involved in a chromosomal rearrangement, based on the observed proximity score of said at least one genomic fragment of the plurality of genomic fragments and the expected proximity score of said at least one genomic fragment of the plurality of genomic fragments and identifying said genomic fragment as a candidate rearrangement partner. Preferred embodiments of step d) are described further herein as embodiments of PLIER.
Preferably, step b) comprises performing oligonucleotide probe hybridization or primer-based amplification to enrich for proximity linked products that comprise genomic fragments comprising sequences flanking the 5′ end of the genomic region of interest and/or step c) comprises performing oligonucleotide probe hybridization or primer-based amplification to enrich for proximity linked products that comprise genomic fragments comprising sequences flanking the 3′ end of the genomic region of interest.
Preferably, step b) comprises providing at least one oligonucleotide probe or primer that is at least partly complementary to sequences flanking the 5′ region of the genomic region of interest, and/or step c) comprises providing at least one oligonucleotide probe or primer that is at least partly complementary to sequences flanking the 3′ region of the genomic region of interest.
Preferably, the method comprises determining the position of the chromosomal breakpoint junction fusing the candidate rearrangement partner to a position within the genomic region of interest, said method comprising:
enriching for proximity linked products that comprise i) at least part of the genomic region of interest and ii) genomic fragments being in proximity to the genomic region of interest sequencing said proximity linked products and mapping the chromosomal breakpoint, wherein the mapping comprises detecting I) proximity linked products comprising at least a first part of the genomic region of interest and genomic fragments of a rearrangement partner and II) proximity linked products comprising at least a second part of the genomic region of interest and genomic fragments of a rearrangement partner, wherein the rearrangement partner genomic fragments from I) and II) are linearly separated.
   Preferably, the method comprises performing oligonucleotide probe hybridization or primer-based amplification to enrich for proximity linked products that comprise i) at least part of the genomic region of interest and ii) genomic fragments being in proximity to the genomic region of interest.
   Preferably, the method comprises generating a matrix for at least a subset of the sequencing reads, wherein one axis of the matrix represents the sequence location of the genomic region of interest and/or the region flanking the genomic region of interest and the other axis represent the sequence location of the candidate rearrangement partner, wherein the matrix is generated by superimposing the sequencing reads over the matrix such that each element within the matrix represents the frequency of a proximity linked product identified that comprises a genomic fragment of the genomic region of interest or flanking the region of interest and a genomic fragment from the rearrangement partner.
   Preferably, the matrix is a butterfly plot.
   Preferably, the method comprises determining the sequence of a genomic region spanning the breakpoint, said method comprising identifying proximity linked products comprising i) breakpoint-proximal genomic fragments of the genomic region of interest and ii) rearrangement partner genomic fragments.
   Preferably, step d) comprises assigning (101) an observed proximity score to each of a plurality of genomic fragments of a genome, the observed proximity score of each genomic fragment being indicative of a presence in the dataset of at least one sequencing read in proximity to the genomic region of interest and comprising a sequence corresponding to the genomic fragment;
   assigning (102) an expected proximity score to each of at least one genomic fragment of the plurality of genomic fragments, based on the observed proximity scores of the plurality of genomic fragments, wherein the expected proximity score comprises an expected value of the proximity score of the at least one of the plurality of genomic fragments; and
   generating (103) an indication of a likelihood that said at least one genomic fragment of the plurality of genomic fragments is involved in a chromosomal rearrangement, based on the observed proximity score of said at least one genomic fragment of the plurality of genomic fragments and the expected proximity score of said at least one genomic fragment of the plurality of genomic fragments and identifying said genomic fragment as a candidate rearrangement partner. Preferred features from step d) are described further herein. For example, in some embodiments, the assigning (102) the expected proximity score to said at least one genomic fragment comprises:
   determining (303) a plurality of related proximity scores based on the observed proximity scores of a plurality of related genomic fragments, wherein the related genomic fragments are related to said at least one genomic fragment according to a set of selection criteria; and
   determining (304) the expected proximity score of said at least one genomic fragment based on the plurality of related proximity scores. Preferably, wherein the determining (303) the plurality of related proximity scores comprises:
   generating (401) a plurality of permutations of the observed proximity scores, thereby identifying a corresponding plurality of permuted observed proximity scores of each of the genomic fragments, wherein generating a permutation comprises swapping the observed proximity scores of randomly chosen genomic fragments that are related to each other according to the set of selection criteria.
   Preferably, wherein
   determining (303) each related proximity score of said at least one genomic fragment further comprises aggregating (402) the permuted observed proximity scores of a permutation by aggregating the permuted observed proximity scores of the genomic fragments in a genomic neighborhood of said at least one genomic fragment within the permutation to obtain an aggregated permuted observed proximity score of the genomic fragment for each permutation.
   further comprising aggregating (101a) the observed proximity scores of the genomic fragments in the genomic neighborhood of said at least one genomic fragment, to obtain an aggregated observed proximity score of said at least one genomic fragment,
   wherein the generating (103) the indication of whether said at least one genomic fragment of the plurality of genomic fragments is involved in a chromosomal rearrangement is performed based on the aggregated observed proximity score of the at least one genomic fragment and the expected proximity score of the at least one genomic fragment. Preferably, further comprising aggregating (101a) the observed proximity scores of the genomic fragments in the genomic neighborhood of each genomic fragment, to obtain an aggregated observed proximity score of each genomic fragment, wherein the permutations are generated (401) based on the aggregated observed proximity score of each genomic fragment, and wherein the generating (103) the indication of whether said at least one genomic fragment of the plurality of genomic fragments is involved in a chromosomal rearrangement is performed based on the aggregated observed proximity score of the at least one genomic fragment and the expected proximity score of the at least one genomic fragment. Preferably, the steps of aggregating the proximity scores (101a), assigning (102) the expected proximity score, and generating (103) the indication of a likelihood that said at least one genomic fragment of the plurality of genomic fragments is involved in a chromosomal rearrangement are iterated (502) for a plurality of different scales (501), wherein in each iteration (101a′, 102′, 103′) a size of the genomic neighborhood is based on the scale. Preferably, determining (304) the expected proximity score of said at least one genomic fragment comprises combining the plurality of related proximity scores of said at least one genomic fragment to determine for example an average and/or a standard deviation. Preferably, the assigning (101) the observed proximity score to each of the plurality of genomic fragments comprises:
   assigning (201) an observed proximity frequency to a plurality of genomic fragments of a genome, the observed proximity frequency being indicative of a presence in the dataset of at least one DNA read of the corresponding genomic fragment; and
   computing (202) each observed proximity score by combining the observed proximity frequencies in a genomic neighborhood of each genomic fragment, for example by binning the observed proximity frequencies. Preferably, the observed proximity frequency comprises a binary value indicating whether the DNA read corresponding to the genomic fragment is present in the dataset or a value indicative of a number of DNA reads corresponding to the genomic fragment in the dataset.
   In some embodiments a method is provided for confirming the presence of a chromosomal breakpoint junction, fusing a candidate rearrangement partner to a position within a genomic region of interest, said method comprising:
defining a genomic region of interest;
performing a proximity assay on a DNA comprising sample to generate a plurality of proximity linked products;
enriching for proximity linked products that comprise genomic fragments comprising sequences flanking the 5′ end of the genomic region of interest,
   wherein said proximity linked products further comprise genomic fragments being in proximity to said genomic fragments comprising sequences flanking the 5′ end of the genomic region of interest; sequencing said proximity linked products to produce sequencing reads,
   mapping to a reference sequence the sequences of the genomic fragments that are in proximity to said genomic fragments comprising sequences flanking the 5′ end of the genomic region of interest;
enriching for proximity linked products that comprise genomic fragments comprising sequences flanking the 3′ end of the genomic region of interest,
   wherein said proximity linked products further comprise genomic fragments being in proximity to said genomic fragments comprising sequences flanking the 3′ end of the genomic region of interest;
   sequencing said proximity linked products to produce sequencing reads, mapping to a reference sequence the sequences of the genomic fragments that are in proximity to said genomic fragments comprising sequences flanking the 3′ end of the genomic region of interest;
enriching for proximity linked products that comprise i) at least part of the genomic region of interest and ii) genomic fragments being in proximity to the genomic region of interest;
   sequencing said proximity linked products to produce sequencing reads, mapping to a reference sequence the sequences of the genomic fragments that are in proximity to the genomic region of interest;
identifying, as a candidate rearrangement partner, at least one genomic fragment based on the proximity frequency of said genomic fragment with the genomic region of interest or genomic fragments comprising sequences flanking the genomic region of interest, (preferred embodiments of this step are described further herein as embodiments of PLIER),
determining whether genomic fragments of the candidate rearrangement partner that are in proximity to said genomic fragments comprising sequences flanking the 5′ end of the genomic region of interest and genomic fragments of the candidate rearrangement partner that are in proximity to said genomic fragments comprising sequences flanking the 3′ end of the genomic region of interest are overlapping or linearly separated,
   wherein linear separation of said candidate rearrangement partner genomic fragments is indicative of a chromosomal breakpoint junction within the genomic region of interest;
mapping the location of the chromosomal breakpoint, comprising detecting I) proximity linked products comprising at least a first part of the genomic region of interest and genomic fragments of a rearrangement partner and II) proximity linked products comprising at least a second part of the genomic region of interest and genomic fragments of a rearrangement partner, wherein the rearrangement partner genomic fragments from I) and II) are linearly separated.
   In some embodiments a computer program product is provided for detecting a chromosomal breakpoint fusing a rearrangement partner to a position within a genomic region of interest, said computer program product comprising computer-readable instructions that, when executed by a processor system, cause the processor system to:
generate a matrix for at least a subset of sequencing reads, wherein the sequencing reads correspond to the sequences of proximity linked products, said products comprising genomic fragments from the genomic region of interest or flanking the region of interest and wherein at least a subset of proximity linked products comprises a genomic fragment of a candidate rearrangement partner,
   wherein one axis of the matrix represents the sequence location of the genomic region of interest and/or region flanking the genomic region of interest and the other axis represent the sequence location of the candidate rearrangement partner, wherein the matrix is generated by superimposing the sequencing reads over the matrix such that each element within the matrix represents the frequency of a proximity linked product that comprises a genomic segment of the genomic region of interest or flanking the region of interest and a genomic segment from the rearrangement partner, and
search the matrix to detect one or more coordinates on the axis representing the sequence location of the genomic region of interest and/or region flanking the genomic region of interest that shows a transition in proximity frequency of the genomic segments from the candidate rearrangement partner.
   In some embodiments, the processor system searches the matrix to detect one or more elements that divides at least a part of the matrix into four quadrants, such that the differences in frequency between adjacent quadrants is maximized and the differences between opposing quadrants is minimized.
   Preferably, the processor system
compares the four quadrants identified and
classifies the chromosomal breakpoint as resulting in a reciprocal rearrangement when two opposing quadrants exhibit minimal difference in frequency and the adjacent quadrants exhibit maximal differences in frequency or classifies the chromosomal breakpoint as resulting in a non-reciprocal rearrangement when a single quadrant exhibits the maximal difference in frequency compared to the other three quadrants.
   Preferably, the computer program product is used in any of the methods disclosed herein.

It would be advantageous to be able to detect chromosomal rearrangements more accurately. To better address this concern, a method of detecting a chromosomal rearrangement involving a genomic region of interest is provided. This method, also referred to herein as “PLIER” (Proximity Ligation-based IdEntification of Rearrangements), comprises:

providing a dataset of DNA reads, obtained from a proximity assay (e.g., a nuclear proximity assay), the dataset comprising DNA reads representing genomic fragments being in proximity (e.g., nuclear/linear/chromosomal proximity) to the genomic region of interest;

assigning an observed proximity score to each of a plurality of genomic fragments of a genome, the observed proximity score of each genomic fragment being indicative of a presence in the dataset of at least one DNA read in nuclear proximity to the genomic region of interest and comprising a sequence corresponding to the genomic fragment;

assigning an expected proximity score to each of at least one genomic fragment of the plurality of genomic fragments, based on the observed proximity scores of the plurality of genomic fragments, wherein the expected proximity score comprises an expected value of the proximity score of the at least one of the plurality of genomic fragments; and

generating an indication of a likelihood that said at least one genomic fragment of the plurality of genomic fragments is involved in a chromosomal rearrangement, based on the observed proximity score of said at least one genomic fragment of the plurality of genomic fragments and the expected proximity score of said at least one genomic fragment of the plurality of genomic fragments.

This method and the preferred embodiments described below are useful for identifying, as a candidate rearrangement partner, at least one genomic fragment based on the proximity frequency of said genomic fragment with the genomic region of interest or genomic fragments comprising sequences flanking the genomic region of interest, as described further herein.

The expected proximity score forms a particularly suitable comparison material to compare to the observed proximity score to identify rearrangements.

The assigning the expected proximity score to said at least one genomic fragment may comprise determining a plurality of related proximity scores based on the observed proximity scores of a plurality of related genomic fragments, wherein the related genomic fragments are related to said at least one genomic fragment according to a set of selection criteria; and determining the expected proximity score of said at least one genomic fragment based on the plurality of related proximity scores. This allows for a context-specific expected proximity score, which may be better suited to detect chromosomal rearrangements.

The determining the plurality of related proximity scores may comprise generating a plurality of permutations of the observed proximity scores, thereby identifying a corresponding plurality of permuted observed proximity scores of each of the genomic fragments, wherein generating a permutation comprises swapping the observed proximity scores of randomly chosen genomic fragments that are related to each other according to the set of selection criteria. The permutations may provide an improved accuracy of the determined expected proximity score.

The determining each related proximity score of said at least one genomic fragment may comprise aggregating the permuted observed proximity scores of a permutation by aggregating the permuted observed proximity scores of the genomic fragments in a genomic neighborhood of said at least one genomic fragment within the permutation to obtain an aggregated permuted observed proximity score of the genomic fragment for each permutation. This helps to make the permuted proximity scores more realistic by reducing outliers. In addition, or alternatively, it allows to determine the expected proximity scores at a certain genomic length scale.

The method may comprise aggregating the observed proximity scores of the genomic fragments in the genomic neighborhood of said at least one genomic fragment, to obtain an aggregated observed proximity score of said at least one genomic fragment, wherein the generating the indication of whether said at least one genomic fragment of the plurality of genomic fragments is involved in a chromosomal rearrangement is performed based on the aggregated observed proximity score of the at least one genomic fragment and the expected proximity score of the at least one genomic fragment. This may help to improve the accuracy of the detection. In addition, or alternatively, it allows to determine the observed proximity scores at a certain genomic length scale, which may be the same genomic length scale used to aggregate the permuted observed proximity scores.

Alternatively, the method may comprise comprising aggregating the observed proximity scores of the genomic fragments in the genomic neighborhood of each genomic fragment, to obtain an aggregated observed proximity score of each genomic fragment, and wherein the permutations are generated based on the aggregated observed proximity score of each genomic fragment, and wherein the generating the indication of whether said at least one genomic fragment of the plurality of genomic fragments is involved in a chromosomal rearrangement is performed based on the aggregated observed proximity score of the at least one genomic fragment and the expected proximity score of the at least one genomic fragment. This is another approach to improve accuracy of the detection and/or determine observed and permuted proximity scores at a certain genomic length scale.

The aggregating the observed proximity scores may be performed according to a length scale, and the aggregating the permuted observed proximity scores may be performed according to the same length scale. This allows to determine the significance score indicative of the rearrangement on a particular length scale.

The steps of aggregating the proximity scores, assigning the expected proximity score, and generating the indication of a likelihood that said at least one genomic fragment of the plurality of genomic fragments is involved in a chromosomal rearrangement may be iterated for a plurality of different scales, wherein in each iteration a size of the genomic neighborhood is based on the scale. This way, a multi-scale approach may be provided, to identify a chromosomal rearrangement across multiple scales.

The determining the expected proximity score of said at least one genomic fragment may comprise combining the plurality of related proximity scores of said at least one genomic fragment to determine for example an average and/or a standard deviation. This may provide a value for the expected proximity score that allows to provide a reliable significance score for the rearrangement detection.

The assigning the observed proximity score to each of the plurality of genomic fragments may comprise assigning an observed proximity frequency to a plurality of genomic fragments of a genome, the observed proximity frequency being indicative of a presence in the dataset of at least one DNA read of the corresponding genomic fragment; and computing each observed proximity score by combining the observed proximity frequencies in a genomic neighborhood of each genomic fragment, for example by binning the observed proximity frequencies. This can improve the result by, for example, averaging out noise in the raw proximity frequency data, such as raw ligation frequency data.

The proximity frequency of a genomic fragment may comprise a binary value indicating whether the DNA read corresponding to the genomic fragment is present in the dataset. This allows for example independently ligated fragments.

The proximity frequency of a genomic fragment may comprise a value indicative of a number of DNA reads corresponding to the genomic fragment in the dataset. This allows for example use of untargeted assays.

The providing the dataset of DNA reads may comprise determining a genomic region of interest in the reference genome; performing a proximity assay to generate a plurality of proximity ligated/linked fragments (also referred to as proximity linked products); sequencing the proximity linked products; mapping the sequenced proximity linked products to a reference genome; selecting a plurality of the sequenced proximity linked products that include a genomic fragment that is mapped to the genomic region of interest; and detecting genomic fragments that are ligated to the genomic region of interest in at least one of the selected sequenced proximity linked products. Preferably, the providing the dataset of DNA reads may comprise determining a genomic region of interest in the reference genome; performing a proximity ligation assay to generate a plurality of proximity ligated fragments; sequencing the proximity ligated fragments; mapping the sequenced proximity ligated fragments to a reference genome; selecting a plurality of the sequenced proximity ligated fragments that include a genomic fragment that is mapped to the genomic region of interest; and detecting genomic fragments that are ligated to the genomic region of interest in at least one of the selected sequenced proximity ligated fragments. These are suitable ways to provide the DNA reads. As described further herein, the proximity assay may comprise enriching for proximity linked products that comprise genomic fragments comprising sequences flanking the 5′ end of the genomic region of interest and enriching for proximity linked products that comprise genomic fragments comprising sequences flanking the 3′ end of the genomic region of interest.

The set of selection criteria for identifying the plurality of related genomic fragments that are related to the genomic fragment may comprise at least one of: whether a candidate related genomic fragment localizes in the reference genome in cis to the same chromosome that also harbors the genomic region of interest; whether the candidate related genomic fragment localizes in the reference genome in cis to a specific part of the same chromosome that also harbors the genomic region of interest; and whether the candidate related genomic fragment localizes in the reference genome in trans to a chromosome that does not harbor the genomic region of interest. These criteria may help to improve the quality of the expected proximity score.

The set of selection criteria for identifying the plurality of related genomic fragments that are related to the genomic fragment may comprise at least one of: whether the candidate related genomic fragment localizes to a genomic part of a same or similar three-dimensional nuclear compartment as the genomic region of interest; whether the candidate related genomic fragment localizes to a genomic part that has a same or a similar epigenetic chromatin profile as the genomic region of interest; whether the candidate related genomic fragment localizes to a genomic part that has a similar transcriptional activity as the genomic region of interest; whether the candidate related genomic fragment localizes to a genomic part that has a similar replication timing as the genomic region of interest; whether the candidate related genomic fragment localizes to a genomic part that has a related density of experimentally created fragments as the genomic region of interest; and whether the candidate related genomic fragment localizes to a genomic part that has a related density of non-mappable fragments or fragment ends as the genomic region of interest. This helps to make the expected proximity score more context-aware. In all these examples, “same or similar” may be assessed based on a set of predetermined matching criteria; for example, a ‘cost function’ or ‘error function’ that is larger for less similar situations, and smaller (closer to zero) for more similar situations.

The set of selection criteria for identifying the plurality of related genomic fragments may comprise a requirement that the proximity score of the candidate related genomic fragment has a value indicative of a non-zero number of DNA reads. This may improve the quality of the significance score indicative of a rearrangement.

The generating the indication of the likelihood that said at least one genomic fragment is related to a chromosomal rearrangement may comprise generating a first indication of the likelihood that said at least one genomic fragment is related to a chromosomal rearrangement using a set of selection criteria excluding the requirement that the proximity score of the candidate related genomic fragment has a value indicative of a non-zero number of DNA reads; generating a second indication of the likelihood that said at least one genomic fragment is related to a chromosomal rearrangement using the set of selection criteria including the requirement that the proximity score of the candidate related genomic fragment has a value indicative of a non-zero number of DNA reads; and generating a third indication of the likelihood that said at least one genomic fragment is related to a chromosomal rearrangement, based on the first indication and the second indication. This combination may allow to derive a more reliable likelihood as compared to performing either one of the proposed methods in isolation.

According to another aspect of the invention, a computer program product may be provided, which may be stored on an intangible computer readable media. The computer program comprises computer-readable instructions that, when executed by a processor system, cause the processor system to:

assign an observed proximity score to each of a plurality of genomic fragments of a genome, the observed proximity score of a genomic fragment being indicative of a presence in a dataset of at least one DNA read corresponding to the genomic fragment, wherein the dataset comprises DNA reads, obtained from a proximity assay (e.g., a nuclear proximity assay), the DNA reads representing genomic fragments being in proximity (e.g., nuclear/linear/chromosomal proximity) to a genomic region of interest;

assign an expected proximity score to each of at least one genomic fragment of the plurality of genomic fragments, based on the observed proximity scores of the plurality of genomic fragments, wherein the expected proximity score is an expected value of the proximity score of the at least one of the plurality of genomic fragments; and

generate an indication of a likelihood that said at least one genomic fragment of the plurality of genomic fragments is involved in a chromosomal rearrangement, based on the observed proximity score of said at least one genomic fragment of the plurality of genomic fragments and the expected proximity score of said at least one genomic fragment of the plurality of genomic fragments.

The methods and computer program described above are preferably applied in a method for confirming the presence of a chromosomal breakpoint junction in order to identify candidate rearrangement partners, as described herein.

The person skilled in the art will understand that the features described above may be combined in any way deemed useful. Moreover, modifications and variations described in respect of the method may likewise be applied to an apparatus or to the computer program product.

### BRIEF DESCRIPTION OF THE DRAWINGS

In the following, aspects of the invention will be elucidated by means of examples, with reference to the drawings. The drawings are diagrammatic and may not be drawn to scale. Throughout the drawings, similar items may be marked with the same reference numerals.

FIG. 1 shows a flowchart illustrating a method of detecting a chromosomal rearrangement.

FIG. 2 shows a flowchart illustrating a method to determine a proximity score for a plurality of DNA fragments.

FIG. 3 shows a flowchart illustrating a method of determining an expected proximity score for at least one DNA fragment.

FIG. 4 shows a flowchart illustrating a method of determining a plurality of related proximity scores for specific genomic fragments.

FIG. 5 shows a flowchart illustrating a method of scale-invariant detection of a chromosomal rearrangement.

FIG. 6 shows an illustrative example of detecting a chromosomal rearrangement using an embodiment of PLIER. A. In a given FFPE-TLC dataset that contains mapped fragments (i.e. proximity-ligation products), B. PLIER initially splits the reference genome into equally spaced genomic intervals and then C. calculates for every interval a “proximity frequency” that is defined by the number of segments within that genomic interval that are covered by at least a fragment (or a proximity-ligation product). D. By Gaussian smoothing of proximity frequencies across each chromosome, E. observed “proximity scores” are calculated to remove very local and abrupt increase (or decrease) in proximity frequencies that are most likely spurious. F. An expected (or average) proximity score and a corresponding standard deviation are estimated for genomic intervals with similar properties (e.g. genomic intervals present on trans chromosomes) by in silico shuffling of observed proximity frequencies across the genome followed by a Gaussian smoothing across each chromosome. H. Finally, a z-score is calculated for every genomic interval using its observed proximity score and the related expected proximity scores and standard deviation thereof. Taken together, PLIER objectively searches for genomic intervals with significantly increased concentrations of captured fragments and considers them as prime candidates for rearrangements.

FIG. 7 shows a block diagram of an apparatus for detecting a chromosomal rearrangement.

FIG. 8A shows a schematic overview of the FFPE-TLC workflow. (1) Through sample fixation, spatially proximal sequences (red) are preferentially crosslinked. Next, paraffin is removed and the sample section is permeabilized to allow enzymes to access the DNA. (2) The DNA is fragmented using NlaIII and then (3) ligated, which results in concatenates of co-localizing DNA fragments. (4) After crosslink reversal and DNA purification, (5) the DNA is subjected to next-generation sequencing library preparation. (6) Sequences of interest are enriched using hybrid capture probes. (7) The prepared library is paired-end Illumina sequenced. B. Genome-wide coverage of fragments retrieved from a typical FFPE-TLC experiment targeting MYC, BCL2 and BCL6. Shown in blue is the coverage seen at the (+/−5 Mb) genomic intervals targeted by the capture probes. The rearranged region to the MYC gene (in green) is identified by the concentration of fragments clustered around the GRHPR gene (chr9:31mb-42mb), shown in red. C. The probe sets used in FFPE-TLC not only retrieve the probe-complementary genomic sequences (in blue), but also mega bases of its flanking sequences (i.e. the proximity-ligation products), shown for MYC (pink), BCL2 (brown) and BCL6 (orange). In case of a rearrangement (MYC-GRHPR in this case), the corresponding capture probes also retrieve fragments originating from the rearrangement partner (GRHPR, in red). This is not the case for regions that do not harbor any rearrangement (e.g. BCL2 in brown or BCL6 in orange), as shown for the GRHPR locus.

FIG. 9. A. Overview of structural variant identification by PLIER. B. Schematic explanation of how butterfly plots of proximity-ligation products (green arches on top of chromosomes) between the target gene and the PLIER-identified rearrangement partner can help distinguish true target rearrangements (breakpoints 1-3, inside the probe targeted region) from non-target rearrangements (breakpoint 4, outside the probe targeted region). In a reciprocal rearrangement inside the target locus, the locus should reveal a 5′ part (section a) that preferentially forms proximity-ligation products with one side of the partner locus and that separates from a 3′ part (section b) that preferentially contacts and ligates the other part of the partner locus. If a breakpoint is present in cis outside the probe-targeted region (breakpoint 4), a 5′ (a) and 3′ (b) part of the target gene cannot be distinguished. C. Three examples of reciprocal rearrangements uncovered by butterfly plots, involving MYC, BCL2 and BCL6, respectively. D. Rearrangements can be non-reciprocal, such that only one part of a target locus fuses to a partner, as exemplified using butterfly plots of MYC, BCL2 and BCL6. E. An example of identified amplification events. Such events are apparent from the elevated number of ligation products that are captured by all target genes (shown for MYC, BCL2 and BCL6 genes).

FIG. 10. A. Circos plots showing the rearrangement partners identified in this study, for translocations with MYC (pink), BCL2 (brown) and BCL6 (orange). Partners found by more than one target gene are indicated in bold. The frequency at which a given partner is found in our study is indicated in parentheses. Additionally, over the circumference of each Circos plot (highlighted in light blue), dots indicate the target genes (i.e. MYC with pink dots, BCL2 with brown dots, BCL6 with orange dots) that are found to be rearranged with each partner in our study. B. Example of a non-reciprocal translocation event that fused the different parts of BLC6 to different genomic partners (chr3 and chr5). C. Example of a complex, three-way rearrangement involving IGH, MYC, BCL2 as well as regions on chr8 and chr10, shown in butterfly plots as well as schematically. D. An example in which both alleles of BCL6 are independently involved in rearrangements. E. Overview of breakpoint positions identified in the MYC locus in our study. Such breakpoints are discerned in base pair resolution by mapping fusion-reads captured by FFPE-TLC.

FIG. 11. A. Overview of PLIER identified rearrangements in diluted samples. Green check marks indicate successful identification of translocations by PLIER without any false-positive calls across the genome. Red crosses indicate failure of PLIER in detecting the rearrangement, either by missing the rearrangement or because of false-positive calls on other regions B. Visualization of ligation products as well as PLIER-computed enrichment scores across dilutions for sample F46 that harbors a BCL2-IGH rearrangement. C. Butterfly visualization of F16 and F221 that were negative for breaks in MYC by FISH. FFPE-TLC revealed that they in fact harbor a MYC rearrangement within the same chromosome. D. Butterfly visualization of three BCL6 rearrangements (F38, F40, F49) that were missed by FISH. In two instances (F38, F40), FISH failed to identify the rearrangements as the percentages of cells with breaks were below threshold. E. In F49, FFPE-TLC revealed that a 1.35 Mb section of the TBL1XR1 locus was inserted into the BCL6 locus. F. BCL6 FISH image of F46 showing no breaks at initial inspection. With hindsight, the zoomed-in view (orange boxes) reveals some split signals (white arrows) that indicate the existence of a translocation, as detected by FFPE-TLC.

FIG. 12. A. Comparison of FISH, Capture-NGS and FFPE-TLC results showing rearrangements identified in MYC, BCL2 and BCL6 genes across 19 samples. Each circle is a sample that is analyzed for rearrangements in a particular gene. Filled-in circles indicate correspondence with FISH diagnosis and empty (red) circles indicate discordance with FISH diagnosis. B. Example of false-negative call by Capture-NGS. As the region around the breakpoint (red arrowhead) lacked capture probes and therefore NGS reads, the breakpoint could not be identified for sample F190. SV identification by FFPE-TLC and PLIER is fusion read independent and correctly called the translocation (z-score of 82.4). C. FFPE-TLC capabilities in detecting translocations even if breakpoints occur far away from the probed regions. Each plot demonstrates this ability for a particular gene for two samples, from left to right: BCL2-IGH (shown for F46 and F73), BCL6-IGL (shown for F37 and F45) and MYC-IGH (shown for F50 and F59). The X-axis in each plot indicates the minimum distance between the last probe and the breakpoint position. The Y-axis shows enrichment scores that are computed by PLIER. In all tested cases, PLIER confidently identifies the translocation. even when the probes are located 50 kb away from the breakpoint. D. Diagram showing the fraction of breakpoint sequences from this study that cannot be mapped uniquely on the reference sequence at varying mapping lengths. E. Example of false positive call by Capture-NGS. Breakpoint spanning reads linking the MYC locus to the X chromosome were found, but no translocation peak was called by PLIER for sample F189. PCR using primers on chrX and sequencing confirmed the integration of a 240 bp fragment from chr8, as shown schematically.

FIG. 13. Comparison between FISH diagnoses and FFPE-TLC results. Quantitative overview of samples with FISH diagnosis horizontally and FFPE-TLC calls (using PLIER) vertically. Note that ‘inconclusive’ FISH results refer to samples carrying an unusual or uneven number of FISH signals.

FIG. 14. Schematic view of read structure in FFPE-TLC samples. FFPE-TLC samples were Illumina sequenced in paired-end mode. Probed fragments (shown in light green) may be represented on one read-end only, or on both reads-ends. Apart from such fragments, proximity-ligation fragments (shown in blue) can be present. Such fragments are recognizable through a restriction site recognition sequence (shown as a vertical line in orange) that links them to the probed fragments. Proximity-ligation fragments may originate from the surroundings of the probed area, or from the neighborhood of the rearranged partner if a rearrangement is present either inside the probed area or in its vicinity. If a rearrangement is present, FFPE-TLC reads can also carry fragments that are produced through fusion of probed (or proximity-ligation) fragments to sequences from the rearranged partner (shown in red). Such reads can depict the rearrangement event in base pair resolution and therefore provide even further detail about the occurred structural variant.

FIG. 15. Example of PLIER calls that are later identified as not relevant using butterfly plots. A. In sample F209 when looking from BLC6, PLIER identified a significant increase of enrichment score around chr10:91mb near the PTEN gene (top plot). However, when looking from PTEN, no reciprocal peak at BCL6 was seen, but ˜4.5 Mb away from BCL6. This observation confirms that the rearrangement did not occur within the region of interest (BCL6 in this case). B. The existence of not relevant cases can be further validated in a butterfly visualization of the same case (i.e. F209 looking from BCL6) that is depicted in the left most butterfly plot. As shown, no transition (or breakpoint) of coverage can be seen. Instead a vertical pattern of coverage is visible. We observed two more cases with similar characteristics. One case was seen in F262 when looking from BCL6 and was very similar to the already described case in F209. The other case was in F233 and also looking from BCL6, but this time the increased vertical coverage was seen around chr10:104. All three cases were therefore considered as not relevant calls of PLIER.

FIG. 16. Overview of breakpoints found in BCL2, BCL6 and IGH using captured fusion-reads in FFPE-TLC.

Fusion-reads in FFPE-TLC can map the occurred breakpoints of rearrangements at base pair resolution. This plot visualizes the identified breakpoints seen from BCL2, BCL6 and IGH MYC? locus, across all samples in our study.

FIG. 17. Dilutions coverage vs. enrichment score

FIG. 18. Probe details

### DETAILED DESCRIPTION OF EMBODIMENTS

Certain exemplary embodiments will be described in greater detail hereinafter, with reference to the accompanying drawings. The matters disclosed in this description and drawing, such as detailed construction and elements, are provided to assist in a comprehensive understanding of the exemplary embodiments. Accordingly, it is apparent that the exemplary embodiments can be carried out without those specifically defined matters. Also, well-known operations or structures are not described in detail, since they would obscure the description with unnecessary detail.

### Definitions

In the following description and examples, a number of terms are used. In order to provide a clear and consistent understanding of the specification and claims, including the scope to be given by such terms, the following definitions are provided. Unless otherwise defined herein, all technical and scientific terms used have the same meaning as commonly understood by one of ordinary skill in the art to which this invention belongs. The disclosures of all publications, patent applications, patents and other references mentioned in this specification are incorporated herein in their entirety by reference.

Methods of carrying out the conventional techniques that may be used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al., Molecular Cloning. A Laboratory Manual, 2nd Edition, Cold Spring Harbor Laboratory Press, Cold Spring Harbor, N.Y., 1989; Ausubel et al., Current Protocols in Molecular Biology, John Wiley & Sons, New York, 1987 and periodic updates; and the series Methods in Enzymology, Academic Press, San Diego.

As used herein, the singular forms “a,” “an” and “the” include plural referents unless the context clearly dictates otherwise. For example, a method for isolating “a” DNA molecule, as used above, includes isolating a plurality of molecules (e.g. 10's, 100's, 1000's, 10's of thousands, 100's of thousands, millions, or more molecules).

The expression “genomic region of interest”, as used herein, refers to a DNA sequence of a chromosome of an organism of which it is desirable to assess (at least part of) its structural integrity. For instance, a genomic region which is suspected of comprising a translocation associated with a disease can be defined as a genomic region of interest. A genomic region of interest may be a single DNA fragment, a gene, a genomic locus containing a gene, a part of a chromosome, etc.

In some embodiments, the genomic region of interest corresponds to a “Topologically associating domain” (TAD). TADs are defined by DNA-DNA interaction frequencies and their boundaries are regions across which relatively few DNA-DNA interactions occur. TADs average 0.8 Mb and may contain several protein-coding genes. The TAD boundaries are generally shared by the different cell types of an organism and are enriched for the insulator binding protein CTCF. The expression of genes within a TAD is somewhat correlated, and thus some TADs tend to have active genes and others tend to have repressed genes (see, e.g., Dixon et al. Nature. 2012 May 17; 485(7398): 376-380).

The term ‘gene’, as used herein, refers to an open reading frame and all genetic elements associated with this open reading frame. These genetic elements may include introns, exons, start codons, stop codons, 5′ untranslated regions, 3′ untranslated regions, terminators, enhancer sites, silencer sites, promoters, alternative promoters, TATA boxes and/or CAAT boxes. In prokaryotic contexts, ‘gene’ may also refer to an operon and may comprise multiple open reading frames. In some embodiments, the genomic region of interest refers to the sequences of a gene starting at the 5′ untranslated region (5′UTR) and ending at the 3′ UTR. Methods for predicting open reading frames as well the genetic elements referred to above are well-known to a skilled person. These methods, also referred to as structural annotation, may utilize a number of different databases and computer algorithms reviewed in Ejigu and Jung (Biology 2020, 9(9), 295; https://doi.org/10.3390/biology9090295).

The expression ‘open reading frame’, as used herein, refers to the genetic elements between and including a start codon and a stop codon.

The expression ‘breakpoint cluster region’, as used herein, also referred to as ‘breakpoint clustering region’, refers to a subsequence of an open reading frame or gene from which it is known by the person skilled in the art that chromosomal rearrangements occur or have occurred in a significant number of patients, organisms or specimens. As is known to a skilled person, some genomic regions comprise several breakpoint cluster regions which may be further defined as major breakpoint cluster regions and minor breakpoint cluster regions.

As used herein, the term “allele(s)” means any of one or more alternative forms of a gene at a particular locus. In a diploid cell of an organism, alleles of a given gene are located at a specific location, or locus (loci plural) on a chromosome. One allele is present on each chromosome of the pair of homologous chromosomes. Thus, in a diploid cell, two alleles and thus two separate (different) genomic regions of interest may exist.

The expression “nucleic acid”, as used herein, may refer to any polymer or oligomer of pyrimidine and purine bases, preferably cytosine, thymine, and uracil, and adenine and guanine, respectively (See Albert L. Lehninger, Principles of Biochemistry, at 793-800, Worth Pub. 1982). The present invention contemplates any deoxyribonucleotide, ribonucleotide or peptide nucleic acid component, and any chemical variants thereof, such as methylated, hydroxy methylated or glycosylated forms of these bases, and the like. The polymers or oligomers may be heterogeneous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The expression “sample DNA”, as used herein, refers to a sample that is obtained from an organism or from a tissue of an organism, or from tissue and/or cell culture, which comprises genomic DNA. Genomic DNA encodes the genome of an organism that is the biological information of heredity which is passed from one generation of an organism to the next. A sample DNA from an organism may be obtained from any type of organism, e.g. micro-organisms, viruses, plants, fungi, animals, humans and bacteria, or combinations thereof. For example, a tissue sample from a human patient suspected of a bacterial and/or viral infection may comprise human cells, but also viruses and/or bacteria. The sample may comprise cells and/or cell nuclei. The sample DNA may be from a patient or a subject who may be at risk or suspected of having a particular disease, for example cancer or any other condition which warrants the investigation of the DNA of the organism.

The expression “crosslinking”, as used herein, refers to reacting DNA at two different positions, such that these two different positions connect to each other as a covalent bond between DNA strands. Two DNA strands may be crosslinked directly using UV-irradiation, forming covalent bonds directly between DNA strands. The connection between the two different positions may be indirect, via an agent, e.g. a crosslinker molecule. A first DNA section may be covalently connected to a first reactive group of a crosslinker molecule comprising two reactive groups, that second reactive group of the crosslinker molecule may be covalently connected to a second DNA section, thereby crosslinking the first and second DNA section indirectly via the crosslinker molecule. A crosslink may also be formed indirectly between two DNA strands via more than one molecule. For example, a typical crosslinker molecule that may be used is formaldehyde. Formaldehyde induces covalent protein-protein and DNA-protein crosslinks. Formaldehyde thus may crosslink different DNA strands to each other via their associated proteins. For example, formaldehyde can react with a protein and DNA, covalently connecting a protein and DNA via the crosslinker molecule. Hence, two DNA sections may be crosslinked using formaldehyde forming a connection between a first DNA section and a protein, the protein may form a second connection with another formaldehyde molecule that connects to a second DNA section, thus forming a crosslink which may be depicted as DNA1-crosslinker-protein-crosslinker-DNA2. In any case, it is understood that crosslinking according to the invention may comprise forming covalent connections (directly or indirectly) between strands of DNA that are in physical proximity of each other. DNA strands may be in physical proximity of each other in the cell, as DNA is highly organized, while being separated from a sequence point of view e.g. by 100 kb. As long as the crosslinking method is compatible with subsequent fragmenting and ligation steps, such crosslinking may be contemplated.

The expression “sample of crosslinked DNA”, as used herein, refers to a sample DNA which has been subjected to crosslinking. Crosslinking the sample DNA has the effect that the three-dimensional state of the genomic DNA within the sample remains largely intact. This way, DNA strands that are in physical proximity of each other remain in each other's vicinity. A “sample of crosslinked DNA” may be formalin fixed and paraffin embedded: it may be a tissue or tumor section or biopsy that is preserved and stored as formalin fixed paraffin embedded (FFPE) material. A “sample of crosslinked DNA” may be a an FFPE sample or tumor sample as routinely collected for pathological studies. A “sample of crosslinked DNA” may also be reconstituted chromatin that has been crosslinked, wherein genomic DNA that has been isolated from a cell (e.g. a tissue sample or a DNA sample) is subjected to chromatin reconstitution or otherwise packaged or coated by proteins or molecules that facilitate crosslinking, and subsequent crosslinking. A sample of crosslinked DNA comprises genomic DNA. The sample may be a derived from cells or tissue samples. In some embodiments, the crosslinked DNA is from crosslinked chromatin from a cell, tissue, or nuclei sample. While in a preferred embodiment the sample is from a human patient, DNA from other organisms may also be used.

The expression “Reversing crosslinking”, as used herein, comprises breaking the crosslinks such that the DNA that has been crosslinked is no longer crosslinked and is suitable for subsequent steps such as ligation, amplification and/or sequencing steps. For example, performing a protease K treatment on a sample DNA that has been crosslinked with formaldehyde will digest the protein present in the sample. Because the crosslinked DNA is connected indirectly via protein, the protease treatment in itself may reverse the crosslinking between the DNA. The protein fragments that remain connected to the DNA may hamper subsequent sequencing and/or amplification. Hence, reversing the connections between the DNA and the amino acids in the protein may also result in “reversing crosslinking”. The DNA-crosslinker-protein connection may be reversed through a heating step for example by incubating at 70° C. As in a crosslinked DNA large amounts of protein can be present, it is often desirable to digest the protein with a protease in addition. Hence, any “reversing crosslinking” method may be contemplated wherein the DNA strands that are connected in a crosslinked sample no longer are connected and become suitable for sequencing and/or amplification.

The expression “Fragmenting DNA”, as used herein, refers to any technique that, when applied to DNA (which may be crosslinked DNA or not), results in DNA “fragments”. Well known techniques to fragment the DNA are sonication, shearing and/or enzymatic restriction, but other techniques can also be envisaged.

The expression “restriction endonuclease” or “restriction enzyme”, as used herein, may be an enzyme that recognizes a specific nucleotide sequence (recognition site) in a double-stranded DNA molecule, and will cleave both strands of the DNA molecule at or near every recognition site, leaving a blunt or a 3′- or 5′-overhanging end. The specific nucleotide sequence which is recognized may determine the frequency of cleaving, e.g. a nucleotide sequence of 6 nucleotides occurs on average every 4096 nucleotides, whereas a nucleotide sequence of 4 nucleotides occurs much more frequently, on average every 256 nucleotides.

The expression “Ligating”, as used herein, involves the concatenation of separate DNA fragments. The DNA fragments may be blunt ended or may have compatible overhangs (sticky overhangs) such that the overhangs can hybridize with each other. The ligation of the DNA fragments may be enzymatic, with a ligase enzyme (i.e. DNA ligase). However, a non-enzymatic ligation may also be used, as long as DNA fragments are concatenated, i.e. forming a covalent bond. Typically, a phosphodiester bond between the hydroxyl and phosphate group of the separate strands is formed.

The expression “Oligonucleotide primers” or “primers” in general, as used herein, refer to strands of nucleotides which can prime the synthesis of DNA. DNA polymerase cannot synthesize DNA de novo without primers. A primer hybridizes to the DNA, i.e. base pairs are formed. Nucleotides that can form base pairs, that are complementary to one another, are e.g. cytosine and guanine, thymine and adenine, adenine and uracil, guanine and uracil. The complementarity between the primer and the existing DNA strand does not have to be 100%, i.e. not all bases of a primer need to base pair with the existing DNA strand. From the 3′-end of a primer hybridized with the existing DNA strand, nucleotides are incorporated using the existing strand as a template (template directed DNA synthesis). We may refer to the synthetic oligonucleotide molecules which are used in an amplification reaction as “primers”.

The expression “oligonucleotide probes” or “probes” in general, as used herein, refers to strands of (modified) RNA and/or (modified) DNA nucleotides, which are complementary to and can hybridize, pulldown and extract the sequences of a genomic region of interest ligated/linked to the fragments that were in proximity in the nucleus to the sequences of a genomic region of interest, as done for example in capture-C, promoter-capture C, Targeted Chromatin Capture (T2C), Tiled-C and promoter-capture Hi-C methods (Hughes et al., 2014; Kolovos et al., 2014; Cairns et al., 2016; Martin et al., 2015; Javierre et al., 2016; Dao et al., 2017; Choy et al., 2018; Mifsud et al., 2015; Montefiori et al., 2018; Jager et al., 2015; Orlando et al., 2018; Chesi et al., 2019; Oudelaar et al., 2019). Modified probes include, e.g., xGen Lockdown Probes (5′-biotinylated oligos).

The term “hybridization” as used herein refers to the binding of two nucleic acid strands through base pairing. Nucleic acid sequences such as from probes and primers preferably have a contiguous sequence (e.g. between 15-100 bp) that is at least 90, 95, or 100% identical to their target sequence. As is known to a skilled person selective or specific hybridization is dependent on, e.g., salt and temperature conditions. Preferably stringent hybridization conditions are used such that a probe or primer binds only to its target sequence.

The expression “primer-based amplification”, as used herein, refers to a polynucleotide amplification reaction, namely, a population of polynucleotides that are replicated from one or more starting sequences, i.e. a primer. A suitable primer may have a sequence length of, for example, 15-30 nucleotides. Amplifying may refer to a variety of amplification reactions, including but not limited to polymerase chain reaction (PCR), linear polymerase reactions, nucleic acid sequence-based amplification, rolling circle amplification, isothermal amplification, and the like. Suitable primer-based amplification methods further include Region-Specific Extraction (RSE) (Dapprich et al. BMC Genomics. 2016; 17: 486), molecular inversion probe circularization (Porreca et al. at Methods 2007 November; 4(11):931-6) and loop mediated isothermal amplification (LAMP) (see, e.g., Notomi et al. Nucleic Acids Res 2000 Jun. 15; 28(12):E63)

The expression “sequencing”, as used herein, refers to determining the order of nucleotides (base sequences) in a nucleic acid sample, e.g. DNA or RNA. Many techniques are available such as Sanger sequencing and “High throughput sequencing” technologies, also referred to in the art as next generation sequencing, such as have been offered by Roche, Illumina and Applied Biosystems, or also referred to in the art as third generation sequencing, as described by David J Munroe & Timothy J R Harris in Nature Biotechnology 28, 426-428 (2010) and such as have been offered by Pacific Biosciences and Oxford Nanopore Technologies, may also be used. Such technologies allow from one sample DNA multiple sequence reads in a single run. For example, the number of sequence reads may range from several hundred up to billions of reads in a single run of a high throughput sequence technology. High throughput sequencing technologies may be performed according to the manufacturer's instructions (as have been provided by e.g. Roche, Illumina or Applied Biosystems). Both long-read and short-read sequencing methods are contemplated herein. The technology may involve the preparation of DNA before carrying out a sequencing run. Such preparation may include ligation of adaptors to DNA. Adaptors may include identifier sequences to distinguish between samples. Depending on the size of DNA that is suitable or compatible with the high throughput sequencing technology used, the DNA that is to be sequenced may be subjected to a fragmenting step. An “adapter” is a short double-stranded oligonucleotide molecule with a limited number of base pairs, e.g. about 10 to about 30 base pairs in length, which are designed such that they can be ligated to the ends of fragments. Adaptors are generally composed of two synthetic oligonucleotides which have nucleotide sequences which are partially complementary to each other. Such adapters may be used in combination with PCR based enrichment strategies and/or for the sequencing of proximity ligated molecules.

The expression “sequencing reads”, as used herein, refers to the piece of DNA that is sequenced (“read”) by a nucleic acid sequencer, such as a massively parallel array sequencer (e.g.,

Illumina or Pacific Biosciences of California). A sequencing read may include a portion of a genomic fragment or proximity ligated molecule. The sequencing reads may be mapped to a reference sequence and/or combined in silico through, for example, alignment, to yield a contiguous sequence. In some embodiments, the methods produce at least 1,000, at least 5,000, or at least 10,000 sequencing reads. The number of sequencing reads may refer to the number of sequencing reads corresponding to proximity ligated molecules comprising sequences flanking the 5′ end of the genomic region of interest; proximity ligated molecules comprising sequences flanking the 3′ end of the genomic region of interest; or both proximity ligated molecules comprising sequences flanking the 5′end and the 3′ end of the genomic region of interest. The number of sequencing reads may also refer to proximity ligated molecules comprising fragments of the genomic region of interest. As is clear to a skilled person, the mapping of such extensive sequencing reads requires the use of computer programs, which are known in the art.

With the terms “aligning” and “alignment”, as used herein, is meant the comparison of two or more nucleotide sequences based on the presence of short or long stretches of identical or similar nucleotides. Methods and computer programs for alignment are well known in the art. One computer program which may be used or adapted for aligning is “Align 2”, authored by Genentech, Inc., which was filed with user documentation in the United States Copyright Office, Washington, D.C. 20559, on Dec. 10, 1991.

The expression “reference genome” (also known as a reference assembly), as used herein, refers to a digital nucleic acid sequence database, assembled by e.g. scientists as a representative example of a species' set of genes. As they are often assembled from the sequencing of DNA from a number of donors, reference genomes do not accurately represent the set of genes of any single person. Instead a reference provides a haploid mosaic of different DNA sequences from each donor. For example, GRCh37, the Genome Reference Consortium human genome (build 37) is derived from thirteen anonymous volunteers from Buffalo, N.Y. Other examples of a reference genome include GRCh19 and CRCh38. As will be appreciated by a skilled person, a reference sequence may also be used in the methods described herein. Suitable reference sequences include a reference genome as well as a subset of sequences from a reference genome.

The expression “independently ligated DNA fragment”, as used herein, refers to a DNA fragment that is ligated to a fragment originating from the genomic region of interest of a given allele of a given cell. In proximity-ligation assays an independently ligated fragment may be PCR amplified prior to sequencing and may therefore be sequenced multiple times. Also, in some proximity ligation methods the proximity ligated products obtained after crosslinking (optional), fragmentation and ligation, may be further fragmented, for example for the purpose of efficient PCR amplification, oligonucleotide bait capture pulldown and/or sequencing, in which case different parts of the same independently ligated fragment may be sequenced. In all such instances that an independently ligated fragment contributes multiple reads to the sequencing dataset, filtering may be performed to generate a dataset that most optimally represents the collection of independently ligated fragments.

The expression “chromosomal rearrangements” or “structural variation”, as used herein, refers to the set of hereditary and somatic genetic aberrations comprising chromosomal deletions, chromosomal inversions, chromosomal duplications and chromosomal translocations, wherein chromosomal deletions and inversions occur within the same chromosome (in cis), chromosomal duplications occur within the same chromosome (in cis) or between two or more different chromosomes (in trans) or result in extra-chromosomal copies of a locus, and wherein translocations occur between two different chromosomes (in trans). Chromosomal rearrangement also includes rearrangements resulting from insertions of foreign DNA, such as transgenes and transposons. In some embodiments, the rearrangement partner is foreign DNA.

The expression ‘reciprocal rearrangement’, as used herein, may refer to an exchange of parts of nonhomologous chromosomes, wherein no genetic elements are lost and wherein genetic elements of one chromosome end up being fused to a second chromosome while genetic elements of the second chromosome end up being fused to the first chromosome, and wherein each chromosome involved in the rearrangement has one breakpoint per rearrangement event. ‘Reciprocal rearrangement’ may alternatively refer to the product as a result of an exchange of parts of nonhomologous chromosomes, wherein no genetic elements are lost and wherein genetic elements of one chromosome end up being fused to a second chromosome while genetic elements of the second chromosome end up being fused to the first chromosome, and wherein each chromosome involved in the rearrangement has at least one breakpoint per rearrangement event. Reciprocal rearrangement may be the result of a natural or artificial process and can be identified in a matrix wherein elements of the matrix represent the proximity frequency of a genomic segment in the genomic region of interest and its rearrangement partner.

The expression ‘non-reciprocal rearrangement’, as used herein, refers to the transfer of genetic elements from one chromosome to another, nonhomologous, chromosome, wherein no genetic elements from the second chromosome are transferred to the first chromosome. ‘Non-reciprocal rearrangement’ may alternatively refer to the product as a result of the transfer of genetic elements from one chromosome to another, nonhomologous, chromosome, wherein no genetic elements from the second chromosome are transferred to the first chromosome. ‘Non-reciprocal rearrangement’ may also refer to the insertion of foreign DNA. Non-reciprocal rearrangement may be the result of a natural or artificial process and can be identified in a matrix wherein elements of the matrix represent the proximity frequency of a genomic segment in the genomic region of interest and its rearrangement partner.

The expression “cis-chromosome”, as used herein, refers to the chromosome that according to the reference genome contains the genomic region of interest. Typically, in proximity-ligation technologies, the independently ligated fragments are most likely to come from the cis-chromosome.

In turn, the independently ligated fragments originating from the cis-chromosome are more likely sequences located in close linear proximity to the genomic region of interest than sequences that locate at larger distances from the genomic region of interest.

The expression “trans-chromosome”, as used herein, refers to any chromosome in the organism of interest that is not cis-chromosome.

The term ‘cis-interaction’, as used herein, refers to the close physical proximity of a genetic element originating from a cis-chromosome with respect to the target element. The term ‘trans-interaction’, as used herein, refers to the close physical proximity of a genetic element originating from a trans-chromosome with respect to the target element.

The expressions “ligation frequency”, “linkage frequency”, “interaction frequency”, and “proximity frequency” of a DNA fragment, as used herein, may refer to the number of ligated/linked fragments of that DNA fragment and a genomic region of interest, or, alternatively, to the number of independently ligated/linked fragments of that DNA fragment and the genomic region of interest. The “ligation frequency”, “linkage”, “interaction frequency”, and “proximity frequency”, may refer to the number of cis- and/or trans-interactions of DNA fragments with a given DNA segment that originate from practical or theoretical restriction digestion of DNA, or may alternatively refer to a value that is an indication of the number of cis- and/or trans-interactions of DNA fragments with a given DNA segment that originate from practical or theoretical restriction digestion of DNA. It may also refer to the number of segments originating from practical or theoretical restriction digestion of DNA, within a given genomic interval, that are covered by at least a ligation product, or to a value representing the number of segments originating from practical or theoretical restriction digestion of DNA, within a given genomic interval, that are covered by at least a linkage product. Typically, in proximity-linkage/ligation technologies, the interaction frequency from cis-interactions is higher than the interaction frequency from trans-interactions. The “ligation frequency”, “linkage frequency”, “interaction frequency”, and “proximity frequency” may also refer to a value that is inherently related to either the number of ligated/linked fragments or the number of independently ligated/linked fragments. For example, a p-value representing the probability that the DNA fragment is ligated to the genomic region of interest may also be considered to be a ligation frequency. Such a p-value may, for example, be calculated using a binomial test. The frequency may be a normalized value of the number of interactions detected. Such normalization may include normalizing for differences between samples, including sample quality; as well as normalizing for GC content, mappability, and restriction site frequency.

The expression “Genomic bin” or “bin”, as used herein, refers to a chromosomal interval, in size typically between 5 kb and 1 Mb and preferably between 10 kb and 200 kb, that can replace the DNA fragment as the unit to which ligation frequencies are assigned. The assignment of a ligation frequency to a given bin relies on an operator (summation, mean, median, minimum, maximum, standard deviation, triangular kernels, Gaussian kernels, half-Gaussian kernels or any other type of weighted and parameterized operators) that aggregates the ligation frequency of the DNA fragments contained within that bin.

The expression “Genomic neighborhood” of a fragment or of a bin, as used herein, refers to a defined linear chromosomal interval surrounding the given fragment or bin in the reference genome. The genomic neighborhood of a fragment or a bin can be between 10 kilobases and 5 mega bases and preferably is between 200 kilobases and 3 mega bases. The genomic neighborhood can also be defined based on the number of fragments surrounding the fragment or bin of interest, in which it typically spans between 50-15 k fragments.

The expression “Observed aggregated ligation score”, as used herein, refers to a score that is given to each fragment or bin according to its own ligation frequency and the ligation frequencies of fragments or bins residing in its genomic neighborhood.

The expression “Expected aggregated ligation score”, as used herein, refers to a dual score (i.e. mean and standard deviation) that is given to each fragment or bin according to a background modelled by in silico permutation and aggregation of the ligation frequencies from the same experiment, to represent for each fragment or bin the most probable observed aggregated ligation score (mean) as well as the corresponding variation (standard deviation).

The expressions “related fragments”, “related bins”, “comparable fragments”, and “comparable bins”, as used herein, refer to fragments or bins that are related according to certain matching criteria. These matching criteria may be predetermined and may depend on the experiment at hand. For example, related fragments of a given fragment may be fragments or bins originating from trans chromosomes, the same trans chromosome, the cis chromosome, or to fragments (or bins with fragments) of similar length, or to fragments (or bins with fragments) of similar crosslinking efficiency, digestion efficiency, ligation efficiency, and/or mapping efficiency, or to fragments or bins with similar epigenetic marks, or to fragments or bins with similar GC content or nucleotide composition or degree of conservation, or to fragments or bins residing in the same spatial nuclear compartment (as determined for example by Hi-C methods), or combinations hereof

The expression “context-aware expected aggregated ligation score”, as used herein, refers to an expected aggregated ligation score generated by permuting related fragments or related bins.

The expression “significance score”, as used herein, refers to a score that may be calculated by comparing the observed aggregated ligation score for each fragment or bin to either the expected aggregated ligation score or the context-aware expected aggregated ligation score.

The expression “nuclear proximity assay”, as used herein, refers to any method that enables identifying the DNA fragments that in the nucleus are in proximity to a genomic region of interest. Examples of nuclear proximity assays are “proximity ligation assays” and nuclear proximity assays that do not rely on proximity ligation. Nuclear proximity may also be referred to as chromosomal proximity or physical proximity. In particular, proximity refers to linear proximity, i.e., proximity along the cis-chromosome.

The expression “proximity ligation assay”, as used herein, refers to an assay that relies on ligation of proximal DNA fragments to identify the DNA fragments that in the nucleus are in proximity to a genomic region of interest. Proximity ligation assays are also known in the field, and may be used herein, as chromosome conformation capture assays and include methods like circular chromosome conformation capture or chromosome conformation capture combined with sequencing (4C) technology (Simonis et al., 2006; van de Werken et al., 2012) and variants of 4C technology (e.g. UMI-4C (Schwartzman et al., 2016) and MC-4C (Allahyar et al., 2018)), Hi-C (Lieberman-Aiden et al., 2009), in situ Hi-C(Rao et al., 2014) and targeted locus amplification (TLA) (de Vree et al., 2014). Proximity ligation methods as referred to herein may also include methods that use complementary oligonucleotide probes (composed of (modified) RNA and/or (modified) DNA nucleotides) for hybridization, pulldown and enrichment of sequences of a genomic region of interest ligated to the fragments that were in proximity in the nucleus to the sequences of a genomic region of interest, as done for example in capture-C, promoter-capture C and promoter-capture Hi-C methods (Hughes et al., 2014; Cairns et al., 2016; Martin et al., 2015; Javierre et al., 2016; Dao et al., 2017; Choy et al., 2018; Mifsud et al., 2015; Montefiori et al., 2018; Jager et al., 2015; Orlando et al., 2018; Chesi et al., 2019). Proximity ligation methods further include methods that use immunoprecipitation, or other protein- or RNA-directed strategies to pulldown and enrich for sequences of interest proximity ligated to a genomic region of interest carrying or associated with that particular protein or RNA molecule, such as ChIA-PET (Li et al., 2012) and Hi-ChIP (Mumbach et al., 2017). Examples of proximity ligated assays and chromosome conformation methods are given in (Denker and de Laat, 2016). Proximity ligation assays can be performed with and without crosslinking prior to ligation (Brant et al., 2016).

Nuclear proximity assays (chromosomal/physical proximity assays) to identify the DNA fragments that in the nucleus are in proximity to a genomic region of interest can also be performed without relying on ligation of proximal DNA fragments to a genomic region of interest: an example of a nuclear proximity assay that is not dependent on ligation but that identifies DNA fragments that in the nucleus are in proximity to a genomic region of interest is SPRITE (split-pool recognition of interactions by tag extension) (Quinodoz et al., 2018).

The term “proximity linked products” as used herein, refers to two or more genomic fragments, in proximity to each other, which are linked. Genomic fragments may be linked directly or indirectly. For example, said genomic fragments may be cross-linked and linkage may be determined based on, e.g., barcodes or tags (e.g., SPRITE). In addition, said genomic fragments may be ligated to each other (e.g., as the result of a proximity ligation assay). Such proximity linked products are referred to herein as proximity ligated products. A skilled person will appreciate that the term proximity ligated products as used herein may also generally include proximity linked products, unless specified otherwise.

The expression “contact profile of the genomic region of interest”, as used herein, refers to the genomic map that visualizes the DNA fragments identified as being in nuclear proximity to the genomic region of interest, plotted on a reference genome.

The expression ‘chromosomal breakpoint junction’ and the term ‘breakpoint’, as used herein, refer to the location on a chromosome or on a chromosomal sequence, where two parts of a chromosome and/or DNA product have been fused together as a result of a natural or artificial process. Particularly relevant breakpoint junctions in the present disclosure are those which do not normally occur in healthy or typical patients, organisms or specimens.

The term ‘matrix’, as used herein, refers to a table of numbers, values or expressions, comprised of two axes. The numbers, values or expressions may be represented by a variety of elements, such as colors or grayscale tones.

The expression ‘butterfly plot’, as used herein, refers to a matrix that displays the distribution of a variable for two populations. For example, one axis of the matrix may represent the sequence location of the genomic region of interest and/or the region flanking the genomic region of interest and the other axis represent the sequence location of a candidate rearrangement partner.

### Embodiments

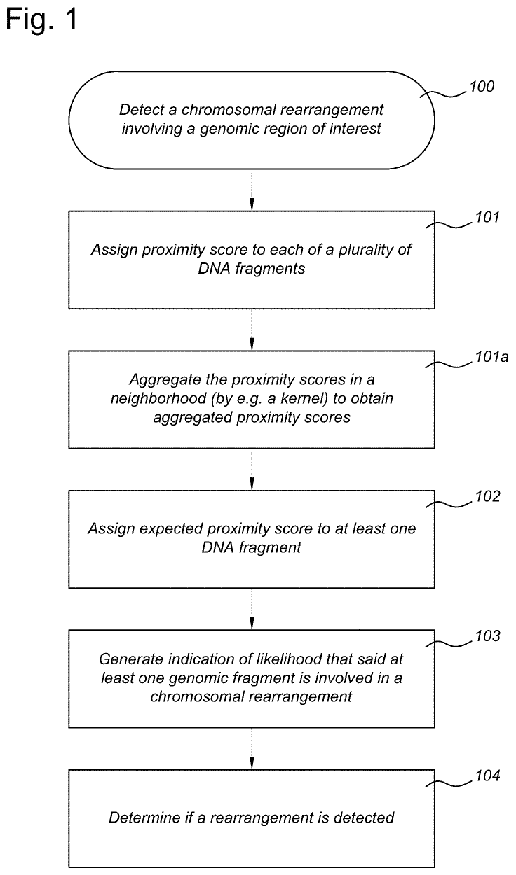

FIG. 1 illustrates a method 100 of detecting a chromosomal rearrangement involving a genomic region of interest. To that end, the method 100 contains a number of steps to analyze a dataset of DNA reads, which may be obtained from a nuclear proximity assay, the dataset comprising DNA reads representing genomic fragments being in nuclear proximity to the genomic region of interest.

The method 100 starts in step 101 with determining a proximity score for each of a plurality of DNA fragments. The proximity score may represent an indication of a likelihood that a DNA fragment is in genomic proximity to a particular genomic region of interest. For example, the proximity score may be related to a collection of DNA reads of fragments that are ligated/linked to a particular genomic region of interest. More generally, the reads are a plurality of reads mapped to DNA fragments that were detected by a detection method to be in close proximity to the genetic region of interest. The proximity score of a DNA fragment indicates the likelihood of that DNA fragment to be in close proximity of region of interest within the nucleus. For example, the proximity score comprises a proximity frequency indicative of a number of reads of that DNA fragment among the reads. Alternatively, the proximity score comprises an indication of whether at least one read of that DNA fragment is present among the reads. Yet alternatively, the proximity score comprises an indication of a likelihood that at least one read of that DNA fragment is present among the reads. For example, the proximity scores can be determined by accessing a database comprising the proximity scores. Moreover, the proximity frequencies may be subjected to a processing step, such as binning, so that the proximity scores relate to bins of genomic fragments.

In aggregation step 101a, the proximity scores of step 101 may be aggregated as another optional step, to obtain aggregated proximity scores. For example, the proximity scores of step 202 may be subjected to a moving average or a weighted moving average along the genome. A weighted moving average may be implemented by convoluting the proximity scores of a genome with a suitable kernel, such as a Gaussian kernel (e.g. sampled Gaussian kernel or discrete Gaussian kernel). This is also called a sliding window approach, which may alternatively involve, for example, sliding Gaussian windows or kernels, half-Gaussian windows or kernels, triangular windows or kernels, rectangular windows or kernels, or other kinds of windows or kernels. The result of the aggregation step 101a may be used as the proximity score of the DNA fragments in step 103. In case the aggregation step 101a is omitted, the proximity score of step 202 may be used, for example.

In step 102, an expected proximity score for at least one DNA fragment is determined. This expected proximity score may be calculated based on the observed proximity scores of the other DNA fragments in the database. For example, an average and standard deviation of all the DNA fragments in the database relating to a particular experiment and/or chromosome may be computed to determine the expected proximity score. Alternatively, a random selection of DNA fragments may be averaged. Yet alternatively, a set of related DNA fragments may be determined, and the proximity scores of only those related fragments may be averaged. The related fragments may be selected based, for example, on their proximity to the genomic region of interest, or on other similarity criteria. Examples of such similarity criteria are disclosed elsewhere in this description.

In step 103, the proximity score of at least one DNA fragment determined in step 101 is compared with the expected proximity score for that at least one DNA fragment. For example, the proximity score of the DNA fragment is compared with the expected proximity score determined in step 102. This results in an indication of a likelihood that the at least one DNA fragment is involved in a chromosomal rearrangement. This indication may be in form of a significance score, for example. In certain implementations, a standard deviation determined in step 102 may be involved in the comparison to determine a statistical significance of any deviation of the observed proximity score versus the expected proximity score. In case a significant deviation is found, a chromosomal rearrangement may be considered to have been detected. The statistical significance may be expressed as a significance score. It will be understood that this significance score may be calculated for each genomic fragment for which both the observed proximity score and the expected proximity score are available.

In step 104, it is decided if a rearrangement is detected. This may be a Boolean decision, i.e., the available significance scores may be evaluated to come at a yes/no decision for each genomic fragment, or the decision may be a soft decision that includes a probability or a likelihood, or a certainty that the genomic fragment is involved in a rearrangement with the genomic region of interest. This decision may be based on the significance score computed in step 103. In certain embodiments, the significance score of step 103 is equal to the soft decision output in step 104.

However, in certain other embodiments, more input variables are taken into account in making the decision, to generate an enhanced significance score indicative of a possible rearrangement. For example, the density of non-mappable experimentally created fragments in the genomic neighbourhood of a mapped target-proximity ligated/linked fragment may be determined. The decision in step 104 may be further based on this density, wherein preferably the enhanced significance score scales positively with the density of non-mappable experimentally created fragments in the genomic neighbourhood of the mapped target-proximity ligated/linked fragment. Moreover, the density of mappable experimentally created fragments in the genomic neighbourhood of a mapped target-proximity ligated/linked fragment may be determined. The decision in step 104 may be further based on this density, wherein preferably the enhanced significance score scales negatively with the expected aggregated proximity score of the given fragment.

After it has been detected at step 104 that there may be a genomic rearrangement involving the particular genomic region of interest and another particular genomic fragment, then the presence of this rearrangement may, optionally, be further verified by performing the whole procedure 100 from the start, using the other particular genomic fragment as “the particular genomic region of interest”. If that procedure confirms the genomic rearrangement, it is even more certain that the rearrangement is real.

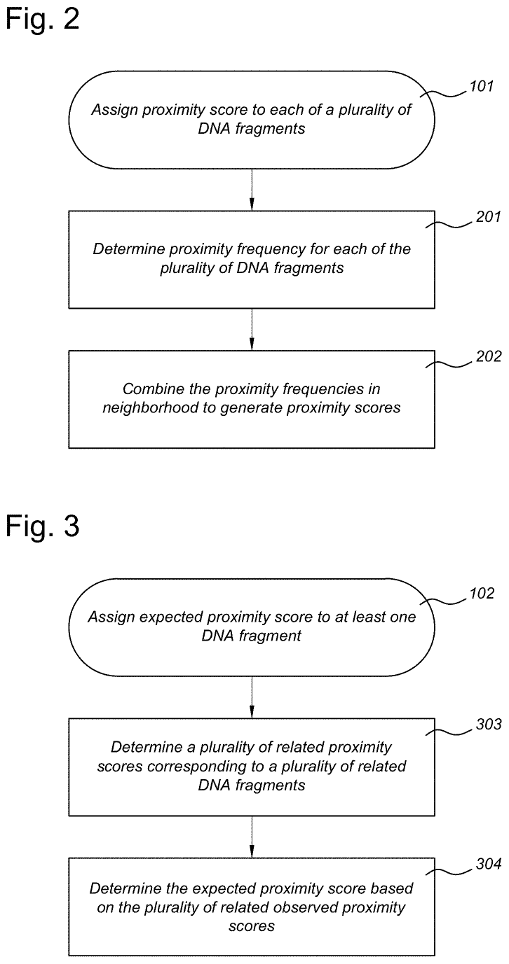

FIG. 2 illustrates a possible method to determine the proximity scores of a plurality of DNA fragments, as performed in step 101 of method 100.

In step 201, a proximity frequency is determined for each of a plurality of DNA fragments. Preferably, a large number of consecutive DNA fragments in the genome is used for this, to facilitate aggregation later on. For example, the proximity frequency of a DNA fragment may be the number of reads of that DNA fragment. Depending on the assay it may be preferable to perform a binarization of the proximity frequency, for example by setting the proximity frequency to 1 if the DNA fragment is found among the reads and 0 if the DNA fragment is not found among the reads.

In step 202, the proximity frequencies of step 201 may be combined as an optional step to generate proximity scores. If step 202 is not performed, the proximity frequencies themselves may be the proximity scores, for example. Step 202 may involve, for example, binning of the proximity frequencies of step 201. For example, bins of a number of consecutive bases each may be defined, and the proximity frequencies may be combined within each bin. The bin size may be chosen, for example, between 5 kilobases and 1 mega base, preferably between 10 kilobases and 200 kilobases. The bins may, for example, have a size of 25 kilobases, although any suitable size of bins may be chosen. The proximity frequencies within each bin may be combined by summing or averaging them, for example. Alternatively, a binomial test may be performed resulting in, for example, a likelihood that the genomic fragments within the bin occur among the reads in the database. Such a binomial test may be particularly suitable in case of binarized proximity frequencies. After binning, the resulting proximity score may be said to be relating to a larger genomic fragment covering the genomic fragments included in the bin.

It will be understood that in certain embodiments, only one aggregation step may be performed (i.e., either the step 202 or the aggregation step 101a, possibly in conjunction with step 402) or no aggregation step at all. However, it may be advantageous to include both aggregation steps. Moreover, in an alternative implementation it is possible to use a kernel filter for the step 202 and binning for the aggregation step 101a.

FIG. 3 illustrates an embodiment of a method implementing step 102 of determining an expected proximity score for at least one DNA fragment. For example, the analysis may be limited to one DNA fragment, or to a certain region within the genome, or to an entire chromosome. Alternatively, the analysis may be performed for the entire genome.

In step 303, a plurality of related proximity scores is generated for each genomic fragment that is to be analyzed. The proximity scores may be the scores resulting from step 101. In this respect, it is noted that a genomic fragment may be considered to be a “bin” of genomic fragments, in case binning is performed in the combining step 202.

In this disclosure, related proximity scores may be proximity scores of genomic fragments that are related to the genomic fragment for which the expected proximity score is being determined. In this regard, genomic fragments may be related to each other when they satisfy certain matching criteria. For example, fragments on the same chromosome may be considered to be related to each other, or fragments within a certain distance on the genome, or fragments known to contribute to a certain function or protein, or fragments that are otherwise comparable. Other matching criteria are disclosed elsewhere in this description. In certain implementations, all the genomic fragments obtained in an experiment are set to be related fragments.

The plurality of related proximity scores may comprise all the proximity scores of the related genomic fragments. Alternatively, for computational efficiency, the collection of related proximity scores may be built up of a random selection of the available related proximity scores. For example, the proximity scores of 1000 (or any other predetermined number of) randomly selected related genomic fragments may be collected.

In step 304, the plurality of related proximity scores are subjected to statistical calculations, so that for example an average and standard deviation are computed as an expected proximity score. Alternatively, for example the median of the related proximity scores may be determined instead of the average, or the variance may be determined instead of the standard deviation. Other statistical methods may be used to calculate an expected proximity score or for example parameters of a probability density function for the proximity score.

This expected proximity score may be calculated for each genomic fragment, as desired.

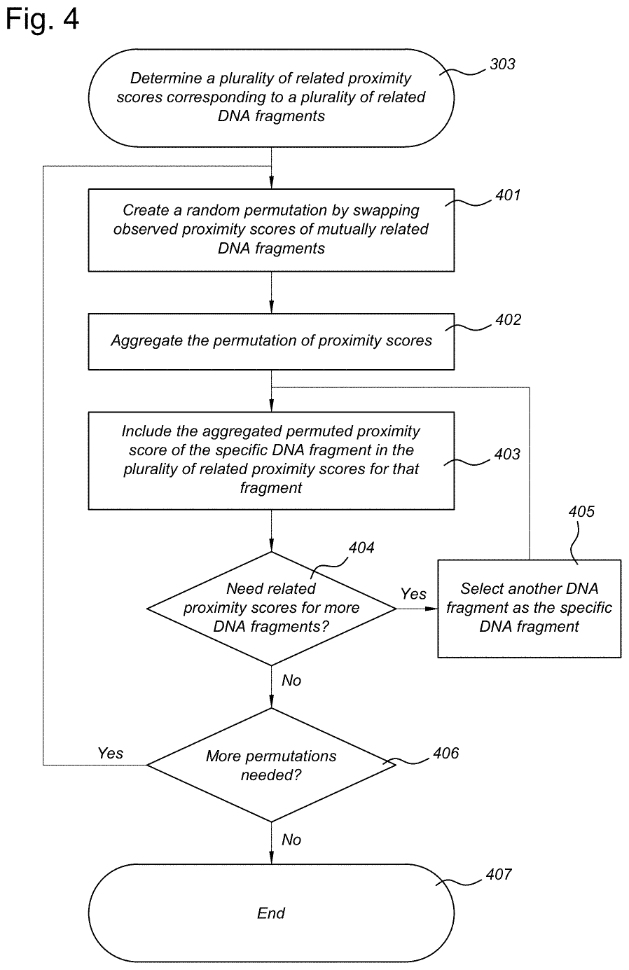

FIG. 4 illustrates an embodiment of a method implementing step 303 of determining a plurality of related proximity scores corresponding to the plurality of related DNA fragments. As observed hereinabove in respect of step 303, the proximity scores determined in step 101 may be used as the starting point for this method.

In step 401, the observed proximity scores of related genomic fragments are permuted. As described above, genomic fragments may be considered to be “related” with each other when they satisfy certain matching criteria. Therefore, in this step, the proximity score of a first fragment may be swapped with the proximity score of a second fragment that is related to the first fragment according to the matching criteria. Each of the proximity scores may thus be swapped with another proximity score. The particular genomic fragments that are swapped may be selected randomly. To create a random permutation, each genomic fragment may be swapped with another randomly chosen related genomic fragment. Alternatively, any number (for example, a fixed number) of swaps between randomly chosen pairs of related genomic fragments may be performed. This step provides permuted proximity scores.

In step 402, the permuted proximity scores of step 401 may be aggregated. Preferably, this aggregation step involves the same operations as the aggregation step 101a that is performed on the observed proximity scores. That way, it is easy to compare the aggregated observed proximity scores to the expected aggregated proximity scores. For example, as discussed above at step 101a, a moving average or a discrete Gaussian kernel may be applied. This step provides aggregated permuted proximity scores.

In step 403, the aggregated permuted proximity scores of step 402 may be collected in a collection associated with a specific DNA fragment, so that later on the expected proximity score may be calculated in step 304. Alternatively, certain statistics corresponding to the specific DNA fragment may be updated based on the aggregated permuted proximity scores of step 402. As illustrated at steps 404 and 405, the aggregated permuted proximity scores of any desired genomic fragments may be collected. This way, the genomic rearrangements/discontinuities may be detected for any number of genomic fragments. In many cases, it may be most useful to collect the aggregated permuted proximity scores of all the genomic fragments on the genome under study.

In step 406, it is decided whether the collection(s) of aggregated permuted proximity scores are sufficiently large. This step may be implemented by an iteration counter, for example. This step may ensure that the expected proximity score will have sufficient statistical relevance. For example, a predetermined number of permutations may be performed; such as 1000 permutations or 100.000 permutations.

If more permutations are needed to enlarge the collections of permuted proximity scores up to the desired number of permutations, in step 406, the process continues from step 401. Otherwise, the collections of related proximity scores are complete at step 407.

It will be understood that in certain embodiments, it is not necessary to store the actual values of the permuted proximity scores in a collection. Instead, it is possible to combine steps 403 and 304 in one step, by updating certain parameters. For example, if only the mean μ and standard deviation σ of the estimated proximity score are desired, it is sufficient to update the sum of the permuted proximity scores Σxᵢ and the sum of squares of permuted proximity scores Σxᵢ², and the number n of permuted proximity scores. After updating these parameters in step 403, the actual values xᵢ of the permuted proximity scores may be discarded. The mean may be calculated afterwards in step 304 as:

$$μ = \frac{\sum {x}_{i}}{n} ,$$

and the standard deviation may be calculated as

$$σ = \sqrt{\frac{\sum {x}_{i}^{2}}{n} - {( \frac{\sum {x}_{i}}{n} )}^{2}} .$$

In certain embodiments, the aggregation steps may implement a length scale. For example, the second aggregation step 101a of the observed proximity scores and the aggregation step 402 of the permuted proximity scores may be used to compare the observed proximity scores to the expected proximity scores at a certain scale. The scale may be considered, for example, the standard deviation of a Gaussian kernel filter, when the aggregation step is implemented by means of a Gaussian filter. Other kinds of filters may have a similar notion of a scale. For example, the window size of a sliding window approach may vary according to the scale. The whole procedure of FIGS. 1 to 4 may be performed a number of times, using different scales. This may lead to different significance findings for different scales. The results for different scales may be combined to obtain a scale-invariant result. For example, the maximum, minimum, or mean of the significance scores obtained from the different scales is used as the final, scale-invariant significance score. Similarly, in certain embodiments, the first aggregation step 202 may be performed at different scales. For example, in case of binning, different bin sizes may be used.

In certain embodiments, the step 101a of aggregating the observed proximity scores in a neighborhood to obtain aggregated proximity scores, and the step 402, of aggregating the permutation of proximity scores, may be performed by processing each DNA fragment as follows. A plurality of neighbor DNA fragments of the DNA fragment is identified. The (observed or permuted) proximity scores of the DNA fragment and the neighbor DNA fragments are selected. The selected proximity scores are combined using an aggregator operator such as a moving average, for example a weighted moving average, for example a Gaussian weighted moving average, or another type of operator along the genome, to produce the aggregated proximity score for the DNA fragment. In certain embodiments, the neighbor DNA fragments may be identified as follows. A distance measure may be chosen to identify neighbor DNA fragments. A first example of a distance measure is a genomic distance. In that case, DNA fragments are selected that are close in terms of genomic length scales, that is, all the fragments less than a certain number of bases (e.g. 200 kilobases or 750 kilobases) away from the DNA fragment may be the neighbor DNA fragments. A second example of a distance measure is the number of DNA fragments along the genome. In that case, the K closest DNA fragments to the DNA fragment may be the neighbor DNA fragments. For example, K=31 or K=51.

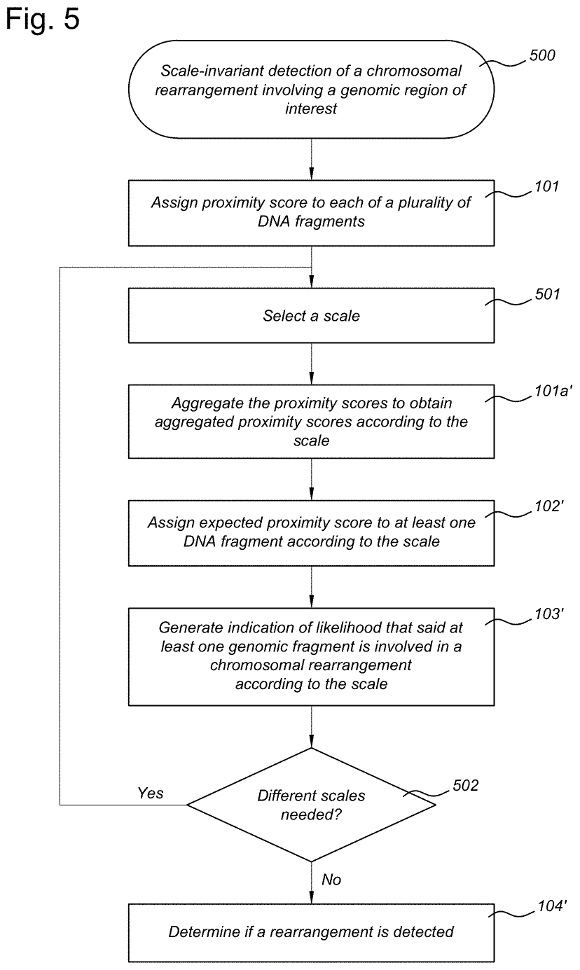

FIG. 5 shows a flowchart of such a scale-invariant detection method of a chromosomal rearrangement involving a genomic region of interest. In FIG. 5, the steps that are similar to the steps of FIG. 1 have been given the same reference numeral as in FIG. 1, provided with an apostrophe. The scale-invariant detection method contains an iteration 502 to determine the significance score in step 103′ at different scales, wherein the scale is set in each iteration in step 501. The final determination of a rearrangement can be made in step 104′ using the significance scores given for the respective scales.

In greater detail, the method starts at step 101 with assigning a proximity score to each of a plurality of DNA fragments in the database with e.g. reads generated by an assay. This step can be identical to step 101 of FIG. 1. An example implementation is shown in FIG. 2.

Next, in step 501, a scale is set. For example, the scale may be expressed as a number of bases. However, this is not a limitation. The scale may be a parameter of an aggregation function that aggregates proximity scores of DNA fragments in a genomic neighborhood. The width of the neighborhood may be determined by the scale. In case the aggregation function is a Gaussian kernel, the scale may be the standard deviation of the Gaussian function used for the Gaussian kernel. The tails of the Gaussian kernel may be optionally cut off at a suitable point. In case the aggregation function is a sliding window, the scale may be the window width of the sliding window. For example, a predetermined set of scales may be selected for the analysis, one scale in each iteration 502. The set of scales can have any number of scales. An example of a set of scales to be used (as e.g. standard deviation or window width) is: {1 kilobase, 1 megabase, 1000 megabases}.

In step 101a′, using the selected scale, the proximity scores are aggregated, using the selected scale as set forth hereinabove. This way, the aggregated proximity scores are obtained. A suitable process for this aggregation step is outlined hereinabove in respect of step 101a.

In step 102′, the expected proximity score for at least one DNA fragment is determined, based on the selected scale. The expected proximity score is assigned to said at least one DNA fragment. The expected proximity score may be assigned to one DNA fragment, for a particular subset of DNA fragments, such as a genomic region, or to the DNA fragments of a whole chromosome or a whole genome. The method to compute the expected proximity score may be implemented, for example, as disclosed hereinabove with reference to FIG. 3 and FIG. 4. In step 402, the permutation of proximity scores may be aggregated using the selected scale. For example, the same aggregation algorithm and aggregation parameters may be used as in step 101a′.

In step 103′, the indication of the likelihood that said at least one genomic fragment is involved in a chromosomal rearrangement, for example a significance score, is determined using the aggregated proximity scores according to the scale of step 101a′ and the expected proximity score according to the scale of step 102′. This way, for each selected scale, a different indication of the likelihood of a chromosomal rearrangement may be obtained.

In step 502, it is verified if all desired scales have been applied. If the calculations are desired for more scales, the process is repeated from step 501, wherein another scale is selected. For example, this process is iterated until all scales of the predetermined set of scales have been selected.

If the process has been performed for all desired scales, the process proceeds in step 104′ to determine if a rearrangement is detected, based on the indications (significance scores) determined in step 103′ for all the selected scales. The indications (significance scores) for the different scales can be combined in one of many possible ways, for example the maximum value, mean value, median value, or minimum value of the available significance scores for the at least one DNA fragment may be determined. A threshold may thereafter be optionally be applied to arrive at a binary determination. After that, the process terminates.

It will be understood that the method described hereinabove with reference to FIGS. 1 to 5 may be implemented as a computer program or as a suitably programmed computer system. The dataset created by means of the proximity assay may serve as the input of such a computer program, and the output may be an indication of a detected rearrangement.

Throughout this disclosure, it may be understood that a ligation frequency is an example of the proximity frequency, and ligation score is an example of proximity score. Although several techniques are illustrated and explained throughout this document using the ligation frequency and ligation score as an example, it will be understood that in general the techniques disclosed herein may be carried out using any proximity frequency and/or proximity score. For example, a nuclear proximity assay may be used that does not rely on “proximity ligation”, such as the SPRITE method, to identify DNA fragments proximal to a genomic region of interest. Therefore, throughout this disclosure, the terms ligation and proximity may be used interchangeably. Specifically, the terms ligation frequency and proximity frequency may be used interchangeably. Similarly, the terms ligation score and proximity score may be used interchangeably.

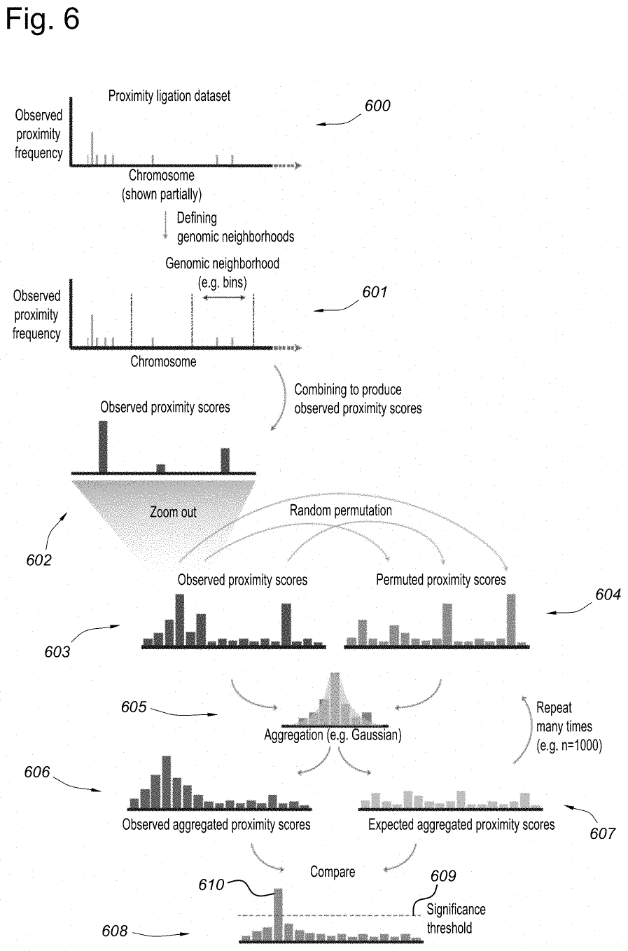

FIG. 6 shows an illustrative example of applying the method set forth herein. As an example, the proximity frequencies can be obtained as a 4C profile or another assay technique. Such an assay may result in a proximity ligation dataset. FIG. 6 shows a graph 600 of the observed proximity frequency (vertical axis) of DNA fragments along a chromosome (shown partially on the horizontal axis). A detail of the graph 600, covering a small portion of the chromosome, is shown in graph 601. The profile is binned using bins having a width of for example 25 kilobases, to obtain a score profile of observed proximity scores. A detail of the score profile is shown in graph 602, and the full score profile is shown in graph 603. The score profile 603 is aggregated using, in this example, a Gaussian kernel 605 to obtain an aggregated, or smoothed, score profile of observed aggregated proximity scores, shown in graph 606. The score profile 603 is permuted to obtain a randomly permuted profile 604, which is also smoothed using the Gaussian kernel 605. The permuting and smoothing are repeated for N times, wherein N is an integer, for example 1000. From all these permuted smoothed profiles, an expected profile of expected aggregated proximity scores is derived, as shown in graph 607. The smoothed profile 606 is compared with the expected profile 607, for example by subtraction (or e.g. by squared difference), to obtain a difference profile, shown in graph 608. A significance threshold 609 is also derived from the permuted smoothed profiles and/or the expected profile. Alternatively, the significance threshold 609 may be set to a configurable value. At a fragment where the comparison profile 608 exceeds the significance threshold 609, as indicated at fragment 610, an indication of a possible rearrangement may be triggered.

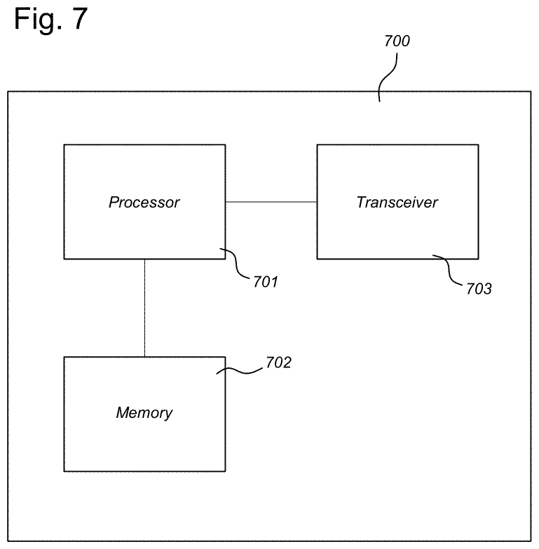

FIG. 7 shows a block diagram of an apparatus for detecting a chromosomal rearrangement. The apparatus may be implemented as a computer system that is configured to perform any method disclosed herein. For example, the steps after having obtained the DNA reads may be performed by the apparatus 700. In particular, the computational steps necessary to detect a chromosomal rearrangement may be performed by the apparatus. For example, the apparatus 700 may comprise a processor 701 that can execute instructions. The processor 701 may comprise a plurality of (sub-) processors that are configured to work cooperatively. The apparatus 700 may further comprise a memory 702, which can be any data storage means, such as a flash memory or a random-access memory, or both. The memory 702 can comprise a non-transitory computer readable media. The memory 702 can store instructions causing the processor 701, when executing the instructions, to perform a method set forth herein. These instructions may collectively form a computer program. The computer program can alternatively be stored on a separate non-transitory computer readable media, such as an optical disc. Further, the memory 702 may be configured to store data relating to an assay, for example a database with DNA reads. The data, such as DNA reads, may be received via a transceiver 703, which may be for example a universal serial bus (USB) or a wireless communication device. Also, the result of the method, for example significance scores indicative of any rearrangement, may be output through the transceiver 703. Peripheral devices may be connected by means of the transceiver 703. Optionally, the apparatus 700 comprises user interface components (not illustrated), such as a display and/or a user input device such as mouse, keyboard, or touch panel. Such user interface components may alternatively be connected via the transceiver 703. Moreover, such user interface components may be used to control the operation of the apparatus and/or output a result of the calculations. The transceiver 703 can also communicate with an external memory, for example. Finally, the apparatus 700 may alternatively be implemented as a distributed computer system that performs a part of the computations or data storage on a cloud server and another part on a client device.

In certain embodiments, nuclear proximity assays known as proximity ligation assays may be used. Moreover, technical and biological biases and variation within and between samples of (crosslinked) DNA may be taken into account to computationally identify structural variation occurring in a genomic region of interest.

In certain embodiments, a method of identifying structural variation occurring in a genomic region of interest may comprise the steps of:
Performing a proximity ligation assay to produce a dataset of independently ligated fragments that are in nuclear proximity to a genomic region of interest.
Using the dataset to assign an observed aggregated ligation score to each fragment.
Using the same dataset to compute a context-aware expected aggregated ligation score for each fragment.
Comparing across different chromosomal length scales the fragment's observed vs. context-aware expected aggregated ligation score and identify per chromosomal length-scale fragments with significantly increased aggregated ligation scores compared to the context-aware expected aggregated ligation score.

In certain embodiments, use is made of a nuclear proximity assay that does not rely on “proximity ligation”, such as the ‘SPRITE’ method, to identify DNA fragments proximal to a genomic region of interest and takes into account technical and biological biases and variation within and between samples of (crosslinked) DNA to computationally identify structural variation occurred in a genomic region of interest, comprising the steps of:
Performing a nuclear proximity assay to produce a dataset of DNA fragments that are in nuclear proximity to a genomic region of interest.
Using the dataset to assign an observed aggregated proximity score to each fragment.
Using the same dataset to compute a context-aware expected aggregated proximity score for each fragment.
Comparing across different chromosomal length scales the fragment's observed vs. context-aware expected aggregated proximity score and identify per chromosomal length-scale fragments with significantly increased aggregated proximity scores.

The techniques disclosed herein are based on the realization that it is desirable to detect chromosomal rearrangements more accurately. This is mainly because in comparison of two given samples (e.g. a diseased and a healthy cell) many differences between proximity-ligation products can be detected that are not instigated by actual structural variations. Furthermore, many transitions from low to high interaction frequencies that can be seen in any proximity-ligation dataset are not caused by structural variations. It is therefore an aspect of the present invention to remedy these shortcomings, to identify genomic structural variations in the genome while accounting for intrinsic technical biases observed in the same dataset.

Translocations (chromosomal rearrangements) underlie different forms of cancer (Schram et al., 2017). They may result in the overexpression of oncogenes or the production of fusion proteins having dysregulated expression or kinase activity. The molecular typing of translocations is routinely performed in the clinic for diagnosis (tumor classification), prognosis, and increasingly also for treatment decisions. For example, non-small cell lung carcinoma's (NSCLC) harboring a translocation in the protein kinase genes ALK and ROS1 are targetable by FDA-approved protein kinase inhibitors (Kwak et al., 2010; Shaw et al., 2014), while potent inhibitors of RET are promising precision medicine drugs for patients with RET translocations (Plenker et al., 2017). Molecular typing of NSCLC tumors (Pisapia et al., 2017) is therefore highly useful to select the optimal treatment and obligatory for stage IV (metastatic) lung cancers in the Netherlands (1000 s per year). Translocation analysis is also performed, among others, for the 1500 patients that are annually diagnosed with diffuse large B-cell lymphoma (DLBCL) and many of the annual ˜700 patients having various forms of sarcomas in the Netherlands.

Already for decades, routine clinical procedure is to store surgically removed tumor biopsies as formalin fixed paraffin embedded (FFPE) specimens. However, DNA or RNA rearrangement detection in FFPE samples is compromised due to the fact that DNA and RNA is crosslinked and fragmented. RNA and DNA-based PCR strategies for rearrangement detection exist but are complicated. First, the breakpoint positions and rearrangement partners of recurrently rearranged genes often differ between patients, which makes it difficult to design PCR primer sets that detect all possible rearrangements. Novel fusion partners are often missed, in which case a conclusive remark regarding rearrangements cannot be formed when a negative result is obtained. Some RNA-based PCR strategies like Archer FusionPlex are agnostic for the rearrangement partner, but again not finding a rearrangement in a heterogeneous tumor biopsy does not rule out its presence. Also, there may be too little RNA or the RNA in FFPE samples can be of too low quality for subsequent analysis of cDNA PCR products. Finally, the so-called position effect rearrangements, that do not create fusions but cause the upregulation of otherwise unaltered oncogenes, are per definition undetectable at the RNA level.

For these reasons, fluorescence in situ hybridization (FISH) is often still the preferred diagnostic method for detecting fusions in FFPE biopsies. FISH however is labor intensive, only partially informative and not always conclusive. Each gene needs to be tested separately in an independent FISH experiment. If the gene of interest promiscuously rearranges with different chromosomal partners, which may be often the case, break-apart FISH (or split-FISH) is used. Split-FISH entails the hybridization of differently colored probes on each side of the target gene: if they break-apart (split), i.e. if they are separated over a larger than expected distance in a given number of cells, the gene is considered to be involved in a translocation, but the rearrangement partner remains obscure. Furthermore, depending on the sample quality and tumor load, FISH may give unclear result. A robust, single, all-in-one, assay that can simultaneously detect rearrangements in all genes of interest, irrespective of their breakpoint location and their translocation partner, is therefore highly desired. Such an assay may be made possible using the rearrangement detection methods disclosed herein.

Methodology for rearrangement detection in DNA samples or crosslinked DNA samples would preferably meet any one or more, ideally all, of the following criteria:

(1) an all-in-one method that enables simultaneous monitoring for rearrangements in all genes relevant for the given disease,

(2) a method that is agnostic for the exact breakpoint position and the rearrangement partner and hence able to find known and new translocations partners,

(3) a method that is sufficiently sensitive to also pick up rearrangements in small (for example, less than 5%) subpopulations of cells, and

(4) a method that provides unbiased detection of rearrangements.

Nuclear proximity assays, such as proximity ligation assays, may be able to meet the first three criteria, as was first illustrated by 4C technology. 4C technology was originally developed by the inventors to study the three-dimensional folding of the genome (Simonis et al., 2006). The method is a variant of 3C technology (Dekker et al, 2002) and allows unbiased genome-wide mapping of all chromosomal segments that are in close proximity to a selected genomic site of interest (the ‘viewpoint sequence’). The technique involves formaldehyde-mediated fixation of cells, which results in crosslinks between physically proximal DNA sequences inside each cell nucleus. Crosslinked DNA is subsequently digested with a restriction enzyme and re-ligated under conditions that favor proximity ligation between crosslinked DNA fragments. Hence, 3C strategies create ligation products between DNA sequences that originally were close together in the nuclear space. In 4C technology, inverse PCR with viewpoint-specific primers is performed on circular ligation products, which results in the amplification of its captured ligation partners; these may subsequently be Illumina sequenced and mapped to the genome to uncover the viewpoint's contact profile.

As expected from polymer physics, irrespective of the 3D conformation, the great majority of 4C captured fragments always originates from the sequences that immediately neighbor the viewpoint on the linear chromosome template. Based on this realization the inventors hypothesized and demonstrated in the past that 4C technology is highly suitable for the detection of chromosomal rearrangements, including translocations, as such chromosomal aberrations disturb the linear chromosome scaffold (Simonis et al., 2009; Homminga et al., 2011). Thus, when a 4C viewpoint is in the vicinity of a rearrangement breakpoint, it will identify the rearrangement and the rearrangement partner based on an altered contact profile of the genomic region of interest (Simonis et al., 2009). The sensitivity of the assay (i.e. its ability to detect translocations also in small subpopulations of cells) increases though when viewpoint and breakpoint are closer together: with viewpoints within 100 kb from the breakpoint, translocations may be readily found even if they are present in less than 5% of the cells (Simonis et al., Nat Methods 2009, and unpublished data). The latter is critical for oncogenetic diagnostics as cancer biopsies are often mixtures of healthy and different clonal cancer cell populations. In summary, 4C offers a sensitive method to investigate whether a candidate gene (e.g. a gene that one would want to monitor for rearrangements in the clinic) is involved in a rearrangement and to identify its rearrangement partner. A further advantage of 4C, as published (Simonis et al., 2009), is that the 4C PCR reaction can easily be multiplexed, implying that the assay can simultaneously monitor multiple genes for rearrangements in each patient sample.

Besides 4C technology, we now know that there are many other proximity ligation methods that based on the same principles can also identify chromosomal rearrangements with a genomic region of interest. Examples of such methodologies are targeted locus amplification (TLA), capture-C or capture-HiC methods, Hi-C and in situ Hi-C, ChIA-PET and Hi-ChIP. In principle, all methods that perform proximity ligation to identify the DNA fragments that are in proximity in the nucleus to a genomic region of interest, enable the detection of chromosomal rearrangements and translocations.

Proximity ligation methods can be used to identify chromosomal rearrangements. State of the art methods aiming to identify structural variations based on proximity ligation methods often rely on visual inspection of the contact profile of the genomic region of interest, to find elsewhere on the genome clustering (or absence of clustering) of DNA fragments proximity ligated to the genomic region of interest in a test sample (e.g. a sample from a patient with a disease) that is clearly different from the clustering of proximity ligated DNA fragments seen at that same genomic locus in a control sample (e.g. a sample from a healthy individual). Examples of translocations and other chromosomal rearrangements found upon such visual inspection of the contact profile of the genomic region of interest are given in (Simonis et al., 2009; de Vree et al., 2014; Harewood et al. 2017 and WO2008084405). In other current experimental designs, nuclear proximity dataset obtained in the test sample produced from disease (e.g. cancer) cells are computationally compared to control nuclear proximity datasets produced from normal (healthy) cells to identify abnormal genomic distributions of nuclear proximity DNA fragments indicative of chromosomal rearrangements (Diaz et al. 2018). Dixon et al. 2018 utilizes an extensive control dataset by combining nuclear proximity datasets produced from nine karyotypically normal cell lines to estimate expected inter-chromosomal interaction frequencies that accounts for elevated interactions of fragments originating from chromosome-ends or small chromosomes. The disadvantage of such a test sample versus control sample correction approach is that it cannot account for sample-specific biases that can easily occur in nuclear-proximity assays such as proximity-ligation assays. For example, the purity, the crosslink-ability, the fragmentation efficiency and (in proximity ligation assays) the ligation efficiency of the sample under study can have a substantial impact on how well the fragments located in the 3D proximity of the genomic region of interest are represented in the produced nuclear proximity dataset. Therefore, correcting these hidden experiment-specific biases is a major obstacle in utilizing nuclear proximity technologies for appraising the structural integrity of susceptible loci, and hence for using these methodologies for clinical applications.

Hence, the current inventors devised strategies for identification of structural variations in the region of interest by accounting for dataset-specific technical as well as experimental biases. These strategies may include building a background model that is computed from the proximity-ligation dataset under investigation (for example, from the test sample obtained from a tumor of a patient) and then utilizing the background model to assess significance of clustering of ligated DNA fragments across the genome of that same test sample. In this data-intrinsic analysis procedure, it may be unnecessary to use a control sample dataset.

The inventors have realized, that fragments that are involved in a structural variation (such as chromosomal rearrangement or translocation) with the region of interest will show higher numbers of independently ligated DNA fragments than would be expected by chance.

Based on the above premise, the involvement of a genomic region of interest in a chromosomal rearrangement may be assessed by means of the method, apparatus, and computer program techniques disclosed herein.

In certain embodiments the involvement of a genomic region of interest in a chromosomal rearrangement may be assessed by:
a. Performing a proximity ligation assay that creates a dataset of independently ligated DNA fragments with a genomic region of interest (also referred to herein as proximity ligated/linked products).
b. Aggregating, for example by summation, the ligation frequency in the genomic neighborhood of each fragment, to assign an “observed aggregated ligation score” to each fragment.
c. Permuting (swapping) the ligation frequency of each DNA fragment (including the DNA fragments with observed ligation frequency equal to zero) by another randomly chosen DNA fragment.
d. Aggregating the permuted ligation frequency of each fragment and its neighbor fragments to compute a randomized aggregated ligation score for each fragment.
e. Repeating step c-d many times (typically n=1000) to form an “expected aggregated ligation score” for each fragment in the dataset.
f. Optionally, set the observed aggregated ligation score of fragments residing nearby the region of interest as zero. These fragments can be located in a chromosomal interval that extends, for example, maximally 10 Mb away from the genomic region of interest. This step f effectively excludes the observed aggregated ligation scores of the genomic region flanking the genomic region of interest, which may likely have a high significance score not because of an involvement in rearrangement but due to linear adjacency to the region of interest in the un-rearranged genome.
g. Comparing the observed aggregated ligation score of each DNA fragment to the expected aggregated ligation score, to identify DNA fragments of high significance (i.e. with significantly larger observed aggregated ligation score than the expected aggregated ligation scores).

In certain embodiments, a process is provided to assess the involvement of the genomic region of interest in a cis-chromosomal rearrangement (e.g. an intra-chromosomal deletion, inversion, or insertion) and a context-aware expected aggregated ligation score is used to account for differences between the expected ligation frequency of fragments originating from cis- vs. trans-chromosomes, by
a. Performing a proximity ligation assay that creates a dataset of independently ligated DNA fragments with a genomic region of interest (also referred to herein as proximity ligated/linked products).
b. Aggregating the ligation frequency of fragments residing in the neighborhood of each fragment in the dataset to form an observed “aggregated ligation score” for each fragment.
c. Permuting the ligation frequency of each fragment originating from cis-chromosome (including the DNA fragments in cis with observed ligation frequency equal to zero) by another randomly chosen fragment originating from the cis-chromosome.
d. Aggregating the permuted ligation frequency of each fragment and its neighbor fragments originating from the cis-chromosome to compute a randomized aggregated ligation score for each fragment originating from cis-chromosome.
e. Repeating steps b-d many times (typically n=1000) to form an expected aggregated ligation score for each fragment in the dataset.
f. Optionally, set the observed aggregated ligation score of fragments residing nearby the region of interest as zero.
g. Comparing the observed aggregated ligation score of each fragment originating from the cis-chromosome to the expected aggregated ligation score, to identify fragments in the cis-chromosome containing the genomic region of interest with high significance (i.e. with significantly increased observed aggregated ligation scores).

In another embodiment, a process is provided to assess the involvement of the genomic region of interest in an inter-chromosomal rearrangement (i.e. a translocation between chromosomes) while using a context-aware expected aggregated ligation score to account for differences between the expected ligation frequency of fragments originating from cis- vs. trans-chromosomes by
a. Performing a proximity ligation assay that creates a dataset of independently ligated DNA fragments with a genomic region of interest (also referred to herein as proximity ligated/linked products).
b. Aggregating the ligation frequency of fragments residing in the neighborhood of each fragment in the dataset to form an observed “aggregated ligation score” for each fragment.
c. Permuting the ligation frequency of each fragment originating from trans-chromosomes (including the DNA fragments in trans with observed ligation frequency equal to zero) by another randomly chosen fragment originating from a trans-chromosome.
d. Aggregating the permuted ligation frequency of each fragment and its neighbor fragments originating from the same trans-chromosome to compute a randomized aggregated ligation score for each fragment originating from a trans-chromosome.
e. Repeating steps b-d many times (typically n=1000) to form an expected aggregated ligation score for each trans DNA fragment in the dataset.
f. Comparing the observed aggregated ligation score of each fragment originating from a trans-chromosome to the expected aggregated ligation score, to identify fragments in the trans-chromosomes with high significance (i.e. with significantly increased observed aggregated ligation scores).

The aggregation of the proximity frequency of neighbor DNA fragments may comprise summation, rolling-mean, rolling-median, minimum, maximum, standard deviation, triangular kernels, Gaussian kernels, half-Gaussian kernels, or any other type of weighted sum, or any other aggregation method, such as average of squared frequency values within a window of DNA fragments around a particular DNA fragment in the genome.

Chromosomal amplifications may typically show relative uniform proximity frequencies across an amplified chromosomal segment. However, rearrangement partners may typically have the highest proximity frequencies near the breakpoint that fuses the partner to the genomic region of interest. Moreover, such rearrangement partners may typically show a smaller proximity frequency for fragments further away from the breakpoint.

In certain embodiments, chromosomal amplifications may be discerned from rearrangement partners by permuting the proximity frequency (e.g. in step c. or step 401) exclusively between fragments that are ligated to the genomic region of interest. That is, only the DNA fragments with a proximity frequency higher than zero are permuted when calculating an expected aggregated proximity score.

In certain embodiments, several of the different calculation methods, as disclosed herein, are performed, to detect a chromosomal rearrangement. To increase the detection accuracy, the outcome of these different calculation methods may be combined. For example, the expected aggregated proximity frequencies may be calculated by using either permutations of DNA fragments including DNA fragments with observed proximity frequency equal to zero, or permutations of exclusively DNA fragments with non-zero observed proximity frequency. However, it is also possible to calculate two versions of the expected aggregated proximity frequency, using both methods, and determine the significance of any deviation from both expected aggregated proximity frequencies, and combine the outcome of both methods. For example, only if both methods lead to a significant deviation, a chromosomal rearrangement may be decided. Alternatively, a likelihood of a chromosomal rearrangement may be determined from both methods, and a final likelihood of a chromosomal rearrangement may be determined by combining the likelihoods of the different applied methods. Such a combined method may be performed, for example, when detecting an inter-chromosomal rearrangement, as disclosed hereinabove.

In certain embodiments, the DNA fragments along the genome may be binned, so that a proximity frequency is detected for a bin of closely related DNA fragments rather than for each DNA fragment individually. In such a case, the permutations may be permutations of bins rather than permutations of individual DNA fragments.

In certain embodiments, the significance score of observed aggregated proximity frequencies of DNA fragments or bins may be computed by comparing the observed aggregated proximity frequency of each DNA fragment or bin to the expected aggregated proximity frequency in view of all DNA fragments or bins considered in the experiment. Such procedure may help mitigating the number of false positive calls.

In certain embodiments, the expected aggregated proximity scores may be context-aware. For example, the permutations of the proximity frequencies of DNA fragments may be restricted to swaps between DNA fragments (or bins) that are related, according to certain criteria. “related fragments” and “related bins” may for example be fragments or bins originating from the same trans chromosome, or be fragments or bins originating from a cis-chromosomal segment that locates at a defined linear distance from the genomic region of interest, or be fragments (or bins with fragments) of similar length, or be fragments (or bins with fragments) of similar crosslinking-, digestion-, ligation and/or mapping efficiency, or be fragments (or bins with fragments) from chromosomal segments with similar crosslinking-, digestion-, ligation and/or mapping efficiency, or be fragments or bins from chromosomal segments with similar epigenetic profiles or similar transcriptional activity or similar replication timing (in the cell type under investigation), or be fragments or bins with similar GC content or nucleotide composition or degree of conservation, or be fragments or bins residing in the same spatial nuclear compartment (for example A and B compartments, as determined for example by Hi-C methods), or combinations hereof. In these criteria, “similar” may be implemented, for example, by setting a maximal difference between the values of the relevant quantity in the two DNA fragments (or bins) that are swapped.

In certain embodiments, different genomic length scales are considered to identify chromosomal rearrangements involving the genomic region of interest, by for example by considering multiple sizes for neighborhood aggregation. For example, the analysis can compute significance scores for three different genomic length scales across genomic neighborhoods that are 200 kb, 750 kb and 3 mb in size. For example, aggregation can involve averaging the proximity frequency of the N nearest DNA fragments, wherein N is an integer corresponding to the genomic length scale. Alternatively, aggregation can involve a weighted sum of the proximity frequencies of neighboring DNA fragments, by applying a kernel. For example, a kernel may correspond to a Gaussian distribution with a standard deviation, wherein the standard deviation corresponds to the genomic length scale. Similarly, other parameterized kernels may be used, wherein the parameter of the kernel may correspond to the genomic length scale.

In certain embodiments, significance scores computed for a plurality of different length scales of genomic neighborhoods may be combined, to produce a “scale-invariant” significance score. The typical operators for significance score combination are minimum and mean, but other operators can be utilized as well.

In certain embodiments, the proximity frequency may be corrected for density of DNA fragments with at least one read mapped to it (k) in the neighborhood of each DNA fragment in a sparse dataset by employing a binomial test that accounts for the total number of fragments in the genome (N), and the chance of a DNA fragment to have at least one read mapped to it

$$( p = \frac{M}{N}$$

where M is total number of DNA fragments having at least one read mapped to it in the dataset). The resulting p-value is then considered as proximity frequency of each fragment (see Eq. 1). The proximity frequency of neighbor fragments is combined into aggregated proximity score.

$$B ( k , N , p ) = P r ( k ; N , p ) = {\sum }_{i = 1}^{k} ( \begin{matrix}n \\ i\end{matrix} ) {{p}^{i} ( 1 - p )}^{n - i} \qquad Eq . 1$$

In certain embodiments, the expected proximity score may be corrected for differences between expected proximity frequency of fragments in cis- vs. trans-chromosomes by employing two independent binomial tests. One of the binomial tests accounts for the total number of cis-fragments in the dataset, and the total number of cis-fragments that are covered by at least one read. The other binomial test accounts for the total number of trans-fragments in the dataset, and the total number of trans-fragments that are covered by at least one read.

### Example of Chromosomal Translocation Detection in the Region of Interest Using Circularized Chromosome Conformation Capture (4C) Data

In this example, a region of interest is selected. The region of interest often encloses an oncogene or tumor suppressor gene and the region is commonly found to be rearranged in a particular type of cancer. Next, a 4C experiment is performed in the region of interest using primers that are designed to flank at least one site that is frequently translocated (Krijger et al. 2019). Optionally, a Unique Molecule Identifier (UMI) can be attached to primers to make sure the ligations are independently captured (Schwartzman et al. 2016). Without the use of UMIs in a 4C(-like) experiment involving PCR amplification of ligation products, the ligation frequencies of fragments are preferably first filtered to remove PCR duplicates, which for example can be done by data binarization in the downstream analysis (i.e. to only distinguish between captured (1) and not captured (0) fragments). Thus, once the produced reads are mapped to the reference genome, the ligation frequency of each fragment can be computed according to the number of reads that are mapped to each fragment. If UMI's are not used, the ligation frequency of fragments that are covered by at least one read are set to one, and the rest are set to zero (i.e. binarization to only consider independently ligated fragments).

The ligation frequency of neighbor fragments may be aggregated, for example by a Gaussian kernel centered on each fragment, to form the observed aggregated ligation score. The neighborhood parameter can be set to 200 kb, 750 kb and 3 mb, or any other suitable value. Herein, kb denotes kilobases and mb denotes mega bases.

Next, the ligation frequency of each fragment originating from cis-chromosome is swapped with another randomly chosen fragment originating from cis-chromosome. In other words, the ligation frequency of a first fragment originating from cis-chromosome is assigned to a second, randomly chosen, fragment originating from cis-chromosome, and the ligation frequency of the second fragment is assigned to the first fragment. By this action, the original ligation frequency of the first fragment and second fragment are overwritten by the ligation frequency of the second fragment and first fragment, respectively.

Similarly, the ligation frequency of each fragment originating from trans-chromosome is swapped with another randomly chosen fragment originating from trans-chromosome.

The swapped ligation frequency of each fragment and its neighbors are aggregated by a Gaussian kernel centered on each fragment to compute a randomized aggregated ligation score for each fragment. The swapping procedure is repeated many times (typically n=1000) to form a collection of expected aggregated ligation scores for each fragment in the dataset. From this collection, a mean and standard deviation for the expected aggregated ligation score can be calculated for each fragment. Finally, the observed aggregated ligation score of each fragment is compared to the mean and standard deviation of the expected aggregated ligation score of the corresponding fragment to calculate a z-score (or a p-value if preferred) for each fragment. The z-score (or p-value) identifies fragments with significantly increased observed aggregated ligation score.

In certain embodiments, a structural variation detection experiment in the region of interest can for example be carried out as follows:
1. Select a region of interest that needs to undergo a structural integrity test.
2. Perform a 4C experiment in the region of interest using primers that are designed to flank site(s) that is/are frequently translocated (Krijger et al. 2019).
3. Optionally, attach UMI to primers to discern independently ligated fragments (Schwartzman et al. 2016).
4. Map the captured reads to the reference genome.
5. Compute the ligation frequency of each fragment according to number of reads that are mapped to each fragment.
6. If UMI's are not used, set the ligation frequency of fragments that are covered by at least one read to one, and the rest of the fragments to zero (i.e. binarization).
7. Aggregate the ligation frequency of neighbor fragments using a Gaussian kernel centered on each fragment to form observed aggregated ligation score. The neighborhood parameter can be set, for example, to 200 kb, 750 kb and 3 mb. However, any desired neighborhood parameter can be considered.
8. Swap the ligation frequency of each fragment originating from cis-chromosome with another randomly chosen fragment originating from cis-chromosome.
9. Swap the ligation frequency of each fragment originating from trans-chromosome with another randomly chosen fragment originating from trans-chromosome.
10. Aggregate the swapped ligation frequency of each fragment and its neighbors using a Gaussian kernel centered on each fragment to compute a randomized aggregated ligation score for each fragment.
11. Repeat the swapping procedure many times (typically n=1000) to form a collection of aggregated ligation scores for each fragment in the dataset.
12. Optionally, set the observed aggregated ligation score of fragments residing nearby the region of interest as zero. The area can be, for example, +/−10 mb away from the region of interest. However, any size of the area may be chosen as desired. This step may be used to exclude the observed aggregated ligation scores that are likely to have high significance scores due to linear adjacency to the region of interest from the analysis.
13. Compute the mean and standard deviation of expected aggregated ligation score for each fragment in the dataset, using the collection of aggregated ligation scores for each fragment in the dataset.
14. Compare the observed aggregated ligation score of each fragment to its mean and standard deviation of expected aggregated ligation score, to calculate a z-score (and/or p-value if preferred).
15. Fragments with z-score above a certain threshold, for example 7, may be considered to be involved in genomic rearrangement with the region of interest. Similarly, fragments with a p-value below a certain threshold, for example 0.1, may be considered to be involved in genomic rearrangement with the region of interest.

### Example of Chromosomal Translocation Detection in the Region of Interest Using Targeted Locus Amplification (TLA) Data

In this example, a region of interest may be selected. The region of interest often encloses an oncogene suppressor or tumor suppressor gene and the region may be commonly found to be rearranged in a particular type of cancer. Next, a TLA experiment is performed in the region of interest using primers that are designed to flank a site that is frequently translocated, or a plurality of sites that are frequently translocated (Hottentot et al. 2017). Once the captured reads are mapped to the reference genome, the ligation frequency of each fragment can be computed according to the number of reads that are mapped to each fragment. The ligation frequency of fragments that are covered by at least one read may be set to one, and the rest may be set to zero (i.e. binarization).

The ligation frequency of neighboring fragments may be aggregated by a Gaussian kernel centered on each fragment to form the observed aggregated ligation score. The neighborhood parameter can be set to 200 kb, 750 kb, 3 mb, or any other value.

Next, the aggregated or unaggregated ligation frequency of a plurality of fragments originating from cis-chromosome is swapped with another randomly chosen fragment originating from cis-chromosome. Similarly, the ligation frequency of a plurality of fragments originating from trans-chromosome is swapped with another randomly chosen fragment originating from trans-chromosome. The swapped ligation frequency of each fragment and its neighbors are aggregated, for example by applying a Gaussian kernel centered on each fragment to compute a randomized aggregated ligation score for each fragment. The swapping procedure is repeated many times (typically n=1000) to form a collection of possible aggregated ligation scores for each fragment in the dataset. From this collection, a mean and standard deviation for the expected aggregated ligation score can be calculated. Finally, the observed aggregated ligation score of each fragment is compared to its respective mean and standard deviation of expected aggregated ligation scores to calculate a z-score (or a p-value if preferred) for each fragment. The z-score (or p-value) identifies fragments with significantly increased observed aggregated ligation score.

In certain embodiments, a structural variation detection experiment in the region of interest can for example be carried out as follows:
1. Select a region of interest that needs to undergo a structural integrity test.
2. Perform a TLA experiment in the region of interest using primers that are designed to flank at least one site that is frequently translocated (Hottentot et al. 2017).
3. Map the captured reads to the reference genome.
4. Set the ligation frequency of fragments that are covered by at least one read to one, and the rest of the fragments to zero (i.e. binarization).
5. Aggregate the ligation frequency of neighbor fragments by means of a Gaussian kernel centered on each fragment to form observed aggregated ligation scores. The neighborhood parameter can be set to 200 kb, 750 kb, 3 mb, or any other value.
6. Swap the ligation frequency of each fragment originating from cis-chromosome with another randomly chosen fragment originating from cis-chromosome.
7. Swap the ligation frequency of each fragment originating from trans-chromosome with another randomly chosen fragment originating from trans-chromosome.
8. Aggregate the swapped ligation frequency of each fragment and its neighbors by a Gaussian kernel centered on each fragment to compute a randomized aggregated ligation score for each fragment.
9. Repeat the swapping procedure many times (typically n=1000) to form an expected aggregated ligation score for each fragment in the dataset.
10. Compute the mean and standard deviation of expected aggregated ligation score for each fragment in the dataset.
11. Set the observed aggregated ligation score of fragments residing nearby the region of interest as zero. The area is typically +/−10 mb away from the region of interest. This excludes the observed aggregated ligation scores that are likely to be elevated due to linear adjacency to the region of interest.
12. Compare the observed aggregated ligation score of each fragment to its mean and standard deviation of expected aggregated ligation score, to calculate a z-score (and p-value if preferred).
13. Fragments with z-score above a certain threshold, for example 7, may be considered to be involved in genomic rearrangement with the region of interest.

### Example of Chromosomal Translocation Detection in the Region of Interest Using Hi-C Data

Hi-C data provides a genome-wide view of the chromatin interactome in the population of cells (Lieberman-Aiden et al. 2009). Instead of depicting 3D interactions that occur between a selected fragment representing the region of interest (the so called “viewpoint”) and any other fragments in the genome (as done in 4C or TLA, also known as one vs. all strategies), Hi-C data represents interactions between each fragment in the genome and any other fragments in the genome (also known as all vs. all). Therefore, Hi-C data could be broken into many regions of interests each of which can be independently analyzed for structural integrity using the techniques disclosed herein. To this end, the Hi-C obtained sequenced reads may be initially mapped to the reference genome. Next, reads that are found to be ligated to the selected region of interest may be selected. Next, using the selected reads, the ligation frequency of each fragment may be computed according to the number of selected reads that are mapped to each fragment.

The ligation frequency of neighbor fragments may be aggregated, for example by a Gaussian kernel centered on each fragment, to form the observed aggregated ligation score. The neighborhood parameter (i.e. the length scale) can be set to 200 kb, 750 kb and 3 mb, but other sizes can also be considered.

Next, the ligation frequency of each fragment originating from cis-chromosome may be swapped by another randomly chosen fragment originating from cis-chromosome. Similarly, the ligation frequency of each fragment originating from trans-chromosome may be swapped by another randomly chosen fragment originating from trans-chromosome. The swapped ligation frequency of each fragment and its neighbors may be aggregated, for example by a Gaussian kernel centered on each fragment, to compute a randomized aggregated ligation score for each fragment.

The above swapping procedure may be repeated many times (typically about n=1000 times) to form a collection of aggregated ligation scores for each fragment in the dataset. From this collection, a mean and standard deviation for the expected aggregated ligation score can be calculated for each fragment. Finally, the observed aggregated ligation score of each fragment is compared to its respective mean and standard deviation of expected aggregated ligation scores to calculate a score, for example a z-score or a p-value, for each fragment. The score identifies fragments with significantly increased observed aggregated ligation score.

In certain embodiments, a structural variation detection experiment in the region of interest can for example be carried out as follows:
1. Perform a Hi-C experiment on cells/tissue of interest (Lieberman-Aiden et al. 2009).
2. Map the sequenced reads to the reference genome.
3. Define the genomic region of interest which is sought to undergo a structural integrity test.
4. Select reads that are found to be ligated to the region of interest.
5. Aggregate the ligation frequency of neighbor fragments, for example by a Gaussian kernel centered on each fragment, to form observed aggregated ligation score. The neighborhood parameter can be set to 200 kb, 750 kb and 3 mb but other similar sizes can also be considered.
6. Swap the ligation frequency of each fragment originating from cis-chromosome is by another randomly chosen fragment originating from cis-chromosome.
7. Swap the ligation frequency of each fragment originating from trans-chromosome by another randomly chosen fragment originating from trans-chromosome.
8. Aggregate the swapped ligation frequency of each fragment and its neighbors, for example by a Gaussian kernel centered on each fragment, to compute a randomized aggregated ligation score for each fragment.
9. Repeat the swapping procedure many times (typically n=1000) to form an expected aggregated ligation score for each fragment in the dataset.
10. Compute the mean and standard deviation of expected aggregated ligation score for each fragment in the dataset.
11. Set the observed aggregated ligation score of fragments residing nearby the region of interest to zero. For example, this applies to a genomic area of typically +/−10 mb away from the region of interest. This optional step may be performed to exclude the observed aggregated ligation scores that are likely to be elevated due to linear adjacency to the region of interest.
12. Compare the observed aggregated ligation score of each fragment to its mean and standard deviation of expected aggregated ligation score, to calculate a score, for example a z-score (and/or p-value if preferred).
   Fragments with a score above a certain threshold, for example a z-score above 7, may be considered to be involved in genomic rearrangement with the region of interest.

### Example of Genome-Wide Chromosomal Translocation Detection Using Hi-C Data

Hi-C data provides a genome-wide view of the chromatin interactome in the population of cells (Lieberman-Aiden et al. 2009). Instead of depicting 3D interactions that occur between a selected fragment representing the region of interest (the so called “viewpoint”) and any other fragments in the genome (as done in 4C or TLA, also known as one vs. all strategies), Hi-C data represents interactions between each fragment in the genome and any other fragments in the genome (also known as all vs. all). Therefore, by modifying the described methods and minor modification, the Hi-C data can be exploited to deliver a complete picture of structural integrity of the entire genome. To this end, the Hi-C obtained sequenced reads may be initially mapped to the reference genome. Next, pairs of ligated fragments can be selected. Next, using the selected fragment pairs, the ligation frequency of each fragment pairs may be computed. This essentially forms a matrix that holds frequency of observing a pair of DNA fragment ligated to each other for every combination of DNA fragment pairs in the genome.

The ligation frequency of neighbor fragments pairs may be aggregated, for example by a 2D Gaussian kernel centered on each fragment pairs, to form the observed aggregated ligation score. The neighborhood parameter (i.e. the length scale) can be set to 200 kb, 750 kb and 3 mb, but other sizes can also be considered.

Next, the ligation frequency of each fragment pair may be swapped by another randomly chosen relevant (see FIG. 4) fragment pair. The swapped ligation frequency of each fragment pair and its neighbors may be aggregated, for example by a Gaussian kernel centered on each fragment pair, to compute a randomized aggregated ligation score for each fragment pair.

The above swapping procedure may be repeated many times (typically about n=1000 times) to form a collection of aggregated ligation scores for each fragment pair in the dataset. From this collection, a mean and standard deviation for the expected aggregated ligation score can be calculated for each fragment pair. Finally, the observed aggregated ligation score of each fragment pair is compared to its respective mean and standard deviation of expected aggregated ligation scores to calculate a score, for example a z-score or a p-value, for each fragment pair. The score identifies fragment pairs with significantly increased observed aggregated ligation score.

In certain embodiments, a structural variation detection experiment can for example be carried out as follows:
1. Perform a Hi-C experiment on cells/tissue of interest (Lieberman-Aiden et al. 2009).
2. Map the sequenced reads to the reference genome.
3. Select ligated fragment pairs.
4. Aggregate the ligation frequency of neighbor fragment pairs, for example by a Gaussian kernel centered on each fragment pair, to form observed aggregated ligation score. The neighborhood parameter can be set to 200 kb, 750 kb and 3 mb but other similar sizes can also be considered.
5. Swap the ligation frequency of each fragment pair with another randomly chosen relevant DNA fragment pair.
6. Aggregate the swapped ligation frequency of each fragment pairs and its neighbors, for example by a 2D Gaussian kernel centered on each fragment pair, to compute a randomized aggregated ligation score for each fragment pair.
7. Repeat the swapping procedure many times (typically n=1000) to form an expected aggregated ligation score for each fragment pair in the dataset.
8. Compute the mean and standard deviation of expected aggregated ligation score for each fragment pair in the dataset.
9. Set the observed aggregated ligation score of fragment pairs residing nearby the region of interest to zero. For example, this applies to a genomic area of typically +/−10 mb away from the region of interest. This optional step may be performed to exclude the observed aggregated ligation scores that are likely to be elevated due to linear adjacency to the region of interest.
10. Compare the observed aggregated ligation score of each fragment pair to its mean and standard deviation of expected aggregated ligation score, to calculate a score, for example a z-score (and/or p-value if preferred).
11. Fragment pairs with a score above a certain threshold, for example a z-score above 7, may be considered to be involved in genomic rearrangement with the region of interest.

### Example of Chromosomal Translocation Detection in the Region of Interest Using Capture Hi-C Data

One can employ a Capture Hi-C experiment (Dryden et al. 2014) or a similar experiment employing capture probes to pulldown and extract the sequences of a genomic region of interest (e.g. spanning an entire gene locus, or the gene locus subdivided into multiple parts) ligated to the fragments that were in proximity in the nucleus to the sequences of a genomic region of interest, to help identifying the probable rearrangement partner and the breakpoint in the genomic region of interest. For example, a reciprocal translocation involving a genomic region of interest will have one part of the region fused to the one derivative chromosome and the other part of the genomic region of interest fused to the other derivate chromosome. As a consequence, the part of the genomic region of interest that is at one side of the rearrangement breakpoint will show significantly increased ligation frequencies at the breakpoint and towards the one side of the fused trans chromosome, while the part of the genomic region of interest that is at the other side of the rearrangement breakpoint will show significantly increased ligation frequencies from the breakpoint towards the other side of the fused trans chromosome. By selectively analyzing, using the techniques disclosed herein, the ligation products of different parts of the genomic region of interest, one can estimate or even determine the breakpoint positions in both rearranged loci.

Once the captured reads are mapped to the reference genome, the ligation frequency of each fragment can be computed according to the number of reads that are mapped to each fragment. If the paired-end sequencing is performed, the sequenced reads can be split into multiple datasets according to the ligated genomic part (or fragments) in the region of interest.

The ligation frequency of neighbor fragments may be aggregated, for example by a Gaussian kernel centered on each fragment, to form the observed aggregated ligation score. The neighborhood parameter can be set to 200 kb, 750 kb and 3 mb, but other sizes can also be considered.

Next, the ligation frequency of each fragment originating from cis-chromosome may be swapped with another randomly chosen fragment originating from cis-chromosome. Similarly, the ligation frequency of each fragment originating from trans-chromosome may be swapped with another randomly chosen fragment originating from trans-chromosome. The swapped ligation frequency of each fragment and its neighbors may be aggregated, for example by a Gaussian kernel centered on each fragment, to compute a randomized aggregated ligation score for each fragment.

The swapping procedure may be repeated many times (for example n=1000 times) to form a collection of permuted aggregated ligation scores for each fragment in the dataset. From this collection, a mean and standard deviation for the expected aggregated ligation score can be calculated.

Finally, the observed aggregated ligation score of each fragment may be compared to its respective mean and standard deviation of expected aggregated ligation scores to calculate a score, such as a z-score or a p-value, for each fragment. This score may identify fragments with significantly increased observed aggregated ligation score.

In certain embodiments, a structural variation detection experiment in the region of interest can for example be carried out as follows:
1. Select a region of interest that needs to undergo structural integrity test.
2. Perform a Capture HiC experiment in the region of interest using set of probes that are designed to cover at least one genomic site that is frequently translocated (Dryden et al. 2014).
3. Map the captured reads to the reference genome.
4. Possibly (in case of paired-end sequencing) split the mapped reads into multiple datasets according to the genomic site of interest they ligated to. Perform the following steps with the dataset of fragments that are ligated to the selected region of interest.
5. Optionally, set the ligation frequency of fragments that are covered by at least one read to one, and the rest of the fragments to zero (i.e. binarization).
6. Aggregate the ligation frequency of neighbor fragments, for example by a Gaussian kernel centered on each fragment, to form an observed aggregated ligation score. The neighborhood parameter can be set to 200 kb, 750 kb and 3 mb but other sizes can also be considered.
7. Swap the ligation frequency of each fragment originating from cis-chromosome with another randomly chosen fragment originating from cis-chromosome.
8. Swap the ligation frequency of each fragment originating from trans-chromosome with another randomly chosen fragment originating from trans-chromosome.
9. Aggregate the swapped ligation frequency of each fragment and its neighbors, for example by a Gaussian kernel centered on each fragment, to compute a randomized aggregated ligation score for each fragment.
10. Repeat the swapping procedure many times (typically n=1000) to form a collection of aggregated permuted ligation scores for each fragment in the dataset.
11. Compute the mean and standard deviation of expected aggregated ligation score for each fragment in the dataset from the collection of aggregated permuted ligation scores.
12. Set the observed aggregated ligation score of fragments residing nearby the region of interest as zero. The area may be, for example, +1-10 mb away from the region of interest. This excludes the observed aggregated ligation scores that are likely to be elevated due to linear adjacency to the region of interest.
13. Compare the observed aggregated ligation score of each fragment to its mean and standard deviation of the expected aggregated ligation score, to calculate a score, such as a z-score and/or p-value if preferred.
14. Fragments with a score above a certain threshold, for example a z-score above 7, may be considered to be involved in genomic rearrangement with the region of interest.
15. In case multiple datasets were created in step 4 (using varied regions of interest), repeat steps 5-14 for at least some of the other datasets with the genomic region of interest that applies to that dataset. Combine the outcome of the different datasets to obtain more detailed information about the rearrangement location.

In the present disclosure, a method is described to process data from a proximity ligation assay in order to detect abnormalities, such as chromosomal rearrangements. The data that is used as the starting point for this analysis method may be a dataset obtained by performing a proximity ligation assay, sequencing the proximity ligated fragments of that proximity ligation assay, and mapping the sequenced proximity ligated fragments to a reference genome.

The starting point for the analysis may thus be a dataset that comprises a plurality of sequenced proximity ligated fragments, mapped to the reference genome. Moreover, a genomic region of interest may be selected according to the application at hand or according to any hypothesis that the user would like to assess.

In certain embodiments, the relationship between proximity score of cis DNA fragments and their linear chromosomal distance to the region of interest in the reference genome is taken into account to more rigorously estimate the expected aggregated ligation score of DNA fragments in the cis chromosome and search for cis-chromosomal rearrangements such as deletions or inversions or insertions, as further detailed below. To this end, for each DNA fragment originating from the cis chromosome, related DNA fragments are probabilistically defined based on their similar linear distance to the region of interest, or based on a non-linear distance function that decreases for further away DNA fragments from the region of interest (Geeven et al. 2018). During the permutation, related DNA fragments are chosen randomly to estimate the expected aggregated ligation score for each DNA fragment in the cis chromosome.

In certain embodiments, genomic insertion into the genomic region of interest (or into sequences proximal to the genomic region of interest) of a DNA sequence originating from elsewhere on the cis chromosome or from a trans-chromosome is detected by searching for DNA fragments from elsewhere on the cis chromosome or from a trans-chromosome with a proximity significance score above a certain threshold.

In certain embodiments, genomic deletion of a DNA sequence involving the genomic region of interest (or sequences proximal to the genomic region of interest) is recognized by initially correcting for the expected aggregated proximity score of DNA fragments in the cis chromosome, and then searching for genomic DNA fragments with a negative significance score below a certain threshold which is indicative for these DNA fragments being deleted. Alternatively, or in addition, the genomic deletion is recognized by searching for genomic DNA fragments with a significance score above a certain threshold, which is indicative for these DNA fragments being located on the opposite side of the deleted part on the cis-chromosome as compared to the genomic region of interest and as a consequence of the deletion brought in closer proximity to the genomic region of interest.

Similarly, genomic inversion of a DNA sequence involving part of the region of interest and sequences proximal to the genomic region of interest is recognized by initially correcting for the expected aggregated ligation score of DNA fragments in the cis chromosome, and then searching for genomic DNA fragments in the cis chromosome of the genomic region of interest that have a positive significance score above a certain threshold that represents the distal end of the inverted genomic region, and genomic DNA fragments in the cis chromosome of the genomic region of interest that have a negative significance score below a certain threshold that represents the proximal end of the inverted genomic region.

In certain embodiments, in order to independently confirm detected structural variations, the estimated significance score of a structural variation on a particular DNA fragment can facilitate the identification of additional evidence for the existence of structural variation, notably by facilitating the finding in the proximity (ligation) dataset of reads that represent at base-pair resolution the fusion of two sequences not neighboring each other in the reference genome.

In certain embodiments, haplotype-specific structural variations can be detected by linking the DNA fragments in the region of interest according to the co-occurring single nucleotide changes within the ligated DNA fragments originating from the region of interest. Using these links, haplotype-specific proximity ligation datasets are formed. Each dataset, is then processed following the disclosed techniques to identify haplotype-specific structural variations.

In certain embodiments, haplotype-specific structural variations can be detected by analyzing the pairs of reads containing the DNA fragments scored as being involved in a structural variation and the DNA fragments from the genomic region of interest they were found proximal to, each for allele-distinguishing genetic variation such that the structural variation can be haplotype resolved.

Some or all aspects of the invention may be suitable for being implemented in form of software, in particular a computer program product. The computer program product may comprise a computer program stored on a non-transitory computer-readable media. Also, the computer program may be represented by a signal, such as an optic signal or an electro-magnetic signal, carried by a transmission medium such as an optic fiber cable or the air. The computer program may partly or entirely have the form of source code, object code, or pseudo code, suitable for being executed by a computer system. For example, the code may be executable by one or more processors.

As described herein, proximity assays, such as proximity ligation assays, are suitable for identifying rearrangements and candidate rearrangement partners. The inventors have realized that the detection of a rearrangement with such assays does not always indicate though that the rearrangement occurs within the genomic region of interest. As a skilled person will appreciate, rearrangements outside of the genomic region of interest are likely not to have functional consequences in regards to the genomic region of interest. As discussed further herein, the inventors realized that the enrichment of proximity linked products comprising genomic fragments flanking the 5′ end and fragments flanking the 3′ end of the genomic region of interest improved the accuracy of identifying chromosomal rearrangements involving breakpoints within the genomic region of interest. Specifically, enrichment strategies may be designed with the aim of minimizing intrinsic noise which in turn supports the downstream analyses to better discern genuine chromosomal rearrangements within the genomic region of interest (“true positive calls”) from chromosomal rearrangements outside the region of interest (“false positive calls”). More importantly, enrichment strategies should be designed such that one can best discern chromosomal rearrangements having the chromosomal breakpoint inside the genomic region of interest from chromosomal rearrangements having the chromosomal breakpoint in cis (on the same chromosome) but outside the genomic region of interest allowing to discern between relevant and non-relevant events.

False positive calls for chromosomal rearrangements can occur for various reasons, one reason being occasional undesired probe or primer hybridization to off-target sequences elsewhere in the genome. As a consequence, off-target proximity ligation products will be enriched, sequenced and mapped and therefore can show accumulation of proximity ligation products on the chromosomal segment carrying the off-target hybridization sequence. Such accumulation of signal may falsely be recognized as having a chromosomal rearrangement (false positive call).

Multiple strategies have been developed to account for this undesired effect. One strategy is to use control individuals that are not expected to carry a rearrangement involving the chromosomal region of interest. Identification of same chromosomal rearrangements in control samples is a sufficient evidence to recognize these calls as false positive. In such instances, the corresponding chromosomal segments covering the rearrangement can be blacklisted. Another strategy to prevent false positive calls for rearrangements arising from off-target probe or primer hybridization and consequent enrichment of off-target chromosomal proximity products is to identify the individual probes or primers that are responsible for off-target hybridization and exclude them, physically or in silico, from the probe or primer panels targeting the chromosomal region of interest.

Another source of false positive calls arises from copy number variations that are present in the genome of the sample under study. Although the underlying biological reason is different from off-target probe or primer hybridization, the genomic segments of the genome that underwent increased copy number variation are likely to show accumulation of proximity linked products. Again, such accumulation of signal may falsely be recognized as a relevant chromosomal rearrangement (false positive call). To remedy this, one can analyze proximity linked datasets from other regions of interest defined on the same sample. To this end, presence of copy number variation can be recognized by querying if the same chromosomal rearrangement is identified from different regions of interest in in the same sample, but is not always sufficient.

As described above, proximity assays can readily detect a chromosomal rearrangement. However, the examples described herein demonstrate that such assays do not always discriminate between events with breakpoint junctions inside the genomic region of interest (relevant) and chromosomal breakpoint junctions outside the genomic region of interest (not relevant). Surprisingly, for many cases where the chromosomal breakpoint is located outside the genomic region of interest, significantly higher than expected nuclear proximity products accumulating on the fused genomic partner were identified leading to the event being detected and called ‘positive’. The examples further demonstrate that such false positive calls can even occur when breakpoints are mega bases away in cis (on the same chromosome) from the region of interest. For many applications, it is crucial to make a distinction between these two scenarios.

There are a large number of genes well-known to the skilled person that when mutated, e.g., as a result of rearrangements, are associated with a disorder, such as cancer. In order for a medical practitioner to accurately diagnose or prognose said disorder, it is important to know where the rearrangement occurs in relation to the genomic region of interest. For example, when searching for fusion genes creating oncogenic fusion gene products, it is preferred to map the chromosomal breakpoint to a location inside the gene. As another example, when searching for a chromosomal rearrangement that may place a proto-oncogene under the influence of novel transcription regulatory DNA sequences that alter its expression level to oncogenic activity levels, it is preferred to map the chromosomal rearrangement breakpoint to a chromosomal location sufficiently close to the proto-oncogene to expect its altered transcriptional regulation.

The inventors have realized that the prior art methods can be improved to provide increased reliability regarding the calling of true “positives”. One aspect of the present disclosure thus provides methods useful for confirming whether a sample (in particular a patient sample, such as a tumor cell sample) comprises a clinically relevant chromosomal rearrangement. The disclosure further provides methods for identifying chromosomal rearrangements that are indicative of a particular disease, prognosis, or predict response to treatment.

The disclosure provides methods for confirming the presence of a chromosomal breakpoint junction that fuses a candidate rearrangement partner to a position within a genomic region of interest. As used herein, confirming the presence of a chromosomal breakpoint junction also refers to detecting the presence of a chromosomal breakpoint junction that fuses a candidate rearrangement partner to a position within a genomic region of interest. Preferably, the methods comprise determining the genomic region of interest in a reference genome. In some embodiments, the genomic region of interest is between 100 bp to 1 Mb, such as from 1 kb to 10,00 kb.

In a preferred embodiment, the genomic region of interest refers to the DNA sequences encoding the open reading frame of a gene. A skilled person will readily appreciate that breakpoint fusions residing within an open reading frame are likely to affect the function of said gene. Depending on the nature of the rearrangement, the rearrangements may lead to, e.g., premature truncations of the protein encoded by the genomic region of interest, fusion proteins comprising a part of the protein encoded by the genomic region of interest and part of a protein encoded by the rearrangement partner, as well as novel proteins comprising at least a part of the protein encoded by the genomic region of interest together with out-of-frame sequences from the rearrangement partner that now code for “neo”-protein sequences.

In a preferred embodiment, the genomic region of interest refers to a gene. A skilled person will readily appreciate that breakpoint fusions residing within a gene sequence are likely to affect the function of said gene. In addition to the effects described above in regards to rearrangements occurring in the open reading frame, rearrangements can also affect, e.g., the expression and/or transcription of mRNA. For example, a chromosomal rearrangement may bring a gene under the influence of novel transcription regulatory DNA sequences that may alter the gene's expression level. The genomic interval spanning sequences with transcription regulatory potential will differ in size per gene. Considering the structural domain, or topologically associating domain (TAD) containing the target gene, as detected by chromosome conformation studies, preferably in the tissue or cell-type of interest may improve efficiency of the assay in detecting relevant chromosomal rearrangements. Structural domains or TADs are chromosomal segments within which sequences preferentially contact each other and they are flanked by boundaries that insulate genes from contacting and being regulated by transcription regulatory sequences outside the domains. Chromosomal breakpoints located outside structural domains are therefore unlikely to impact expression of the target gene. If structural domains or TADs are undefined, one can define the genomic region of interest, e.g., as the one mega base upstream and the one mega base downstream of the target gene's promoter, since very few transcription regulatory sequences can act over distances further than one mega base. A skilled person is also aware that transcription regulatory sequences may be further away from a gene when in the context of a gene desert (i.e., a genomic interval with no or very few genes surrounding the target gene). Gene deserts typically contain transcription regulatory sequences that can act over large distances on linearly isolated genes.

Preferably, a genomic region of interest is a subsequence of a gene or open reading frame in which rearrangements are known to occur to the person skilled in the art. For example, the genomic region of interest preferably refers to a breakpoint cluster region. Such clusters are well-known in the art. In particular, a skilled person is aware of potential breakpoint clusters associated with a particular disorder. In some embodiments, the methods are suitable for determining whether a rearrangement occurs within breakpoint clusters associated with a particular disorder. An example of a breakpoint cluster region is the 175 bp-long 3′ most exon in the region encoding the 3′ UTR of the BCL2 gene on chromosome 18 in humans, which accounts for 50% of all breaks at the BCL2 gene (Tsai & Lieber, BMC genomics (2010) 11:1). Another example of a breakpoint cluster region is the 7466 bp-long chromosomal region between and including exon 9 and exon 13 of the MLL gene on chromosome 11 in humans (Burmeister et al., Leukemia (2006) 20, 451-457).

The method comprises performing a proximity assay to generate a plurality of proximity linked products. In some embodiments, the assay is a proximity ligation assay to generate a plurality of proximity ligated molecules (see, e.g., FIG. 1). Such proximity ligation assays are described further herein. In an exemplary proximity ligation assay, crosslinked DNA (e.g., formaldehyde crosslinked) is digested with a restriction enzyme and re-ligated under conditions that favor proximity ligation between crosslinked DNA fragments in order to generate proximity ligated molecules. The crosslinking is preferably reversed after ligation.

In some embodiments the proximity ligation assay comprises

a) providing a sample of cross-linked DNA;

b) fragmenting the crosslinked DNA;

c) ligating the fragmented crosslinked DNA to obtain proximity ligated molecules;

d) reversing the crosslinking;

e) optionally fragmenting the DNA of step d), (e.g., with a restriction enzyme or sonication). In some embodiments, the method further comprises

f) ligating the fragmented DNA of step d) or e) to at least one adaptor and

g) amplifying the ligated DNA fragments of step d) or e) comprising the target nucleotide sequence using at least one primer which hybridizes to the target nucleotide sequence, or amplifying the ligated DNA fragments of step f) using at least one primer which hybridizes to the target nucleotide sequence and at least one primer which hybridizes to the at least one adaptor.

Preferably, the method comprises providing a sample of crosslinked DNA for the proximity assay.

In some embodiments, the method comprises enriching for proximity linked products that comprise genomic fragments comprising the genomic region of interest or sequences flanking the genomic region of interest. The skilled person is aware of a number of various targeted DNA enrichment strategies. Generally, such methods rely on the hybridization of an oligonucleotide (such as a probe or primer) to the sequence of interest.

In one embodiment, the method comprises enriching for proximity linked products that comprise genomic fragments comprising sequences flanking the 5′ end of the genomic region of interest and enriching for proximity linked products that comprise genomic fragments comprising sequences flanking the 3′ end of the genomic region of interest. The proximity linked products may be sequenced to produce sequencing reads the sequences of the genomic fragments that are in proximity to said genomic fragments comprising sequences flanking the 5′ or 3′end of the genomic region of interest may be mapped to a reference sequence. “Flanking sequences” refers to sequences which are adjacent to the region of interest. Flanking sequences may be directly or indirectly adjacent to the region of interest.

In one embodiment, the method comprises providing at least one oligonucleotide probe or primer that is at least partly complementary to sequences flanking the 5′ region of the genomic region of interest, and/or providing at least one oligonucleotide probe or primer that is at least partly complementary to sequences flanking the 3′ region of the genomic region of interest. In some embodiments, the probes and primers are complementary to unique target sequences in order to prevent hybridization to repetitive DNA. The oligonucleotide probes can be attached to a solid surface or contain a tag such as biotin that allows capture on a solid surface such as streptavidin beads. In some embodiments, adapter sequences may be ligated to the fragmented DNA. PCR amplification may then be used with one primer complementary to a sequence flanking the genomic region of interest and the other primer complementary to the adapter sequence. Alternatively, or in addition to, the adapter sequences may be used for generating sequencing reads. Probe and primer design is well known to a skilled person. Preferably, oligonucleotide probes and primers are complementary to a sequence between 1 bp to 1 Mbp upstream or downstream from the genomic region of interest. Alternatively, flanking may refer to genomic regions or sequences distant by 0.5% of the length of the chromosome at issue or less. In some embodiments, a panel of probes/primers flanking the genomic region of interest may be used.

The methods further comprise identifying, as a candidate rearrangement partner, at least one genomic fragment based on the proximity frequency of said genomic fragment with the genomic region of interest or sequences flanking the genomic region of interest. As described further herein, the methods may comprise enriching for proximity linked products that comprise i) at least part of the genomic region of interest and ii) genomic fragments being in proximity to the genomic region of interest. Preferably, the method enriches for at least one part of the genomic region of interest. While the presence of the breakpoint junction within the genomic region of interest is confirmed by enriching for proximity ligated molecules comprising sequences flanking the genomic region of interest, the identification of candidate rearrangement partners can be performed based on sequencing reads comprising either the genomic region of interest or sequences flanking the genomic region on interest.

In exemplary embodiments, proximity assays may be targeted to specific genomic regions of interest by the use of complementary oligonucleotide probes for the pulldown and enrichment of nuclear proximity products involving the genomic region of interest. Alternatively, chromosomal proximity assays may be targeted to specific genomic regions of interest by the use of complementary oligonucleotide primers (primers) for the linear or exponential amplification and enrichment of chromosomal proximity products involving the genomic region of interest. Following enrichment, proximity products are sequenced and the sequence reads are mapped to a reference genome. Chromosomal rearrangements are found based on the identification of genomic segments elsewhere in the genome showing a significantly higher than expected accumulation of nuclear proximity products involving the genomic region of interest.

Suitable methods for identifying a candidate rearrangement partner based on proximity frequency are known in the art and are described herein. For example, visual inspection of the contact profile of the genomic region of interest may be used (see, e.g., Simonis et al., 2009; de Vree et al., 2014; and WO2008084405). See, e.g., Harewood et al. for a method based on selecting the top 1% highly interacting intra-chromosomal regions (Genome Biology 2017 18: 125). See also the methods described in Diaz et al. 2018 and Dixon et al. 2018, described herein. Other methods include SALSA, GOTHiC, HiCcompare, HiFI, V4C, LACHESIS, HINT, bin3C. Mifsud describes a model (GOTHiC) to identify true interactions from proximity-ligation data and also reviews other well-known models for identifying rearrangements partners (PLOS ONE 2017 12(4): e0174744).

A preferred method for identifying candidate rearrangement partners is exemplified in FIG. 1-6 and is referred to herein as PLIER. In some embodiments, the method of identifying one or more candidate rearrangement partners includes

selecting a plurality of sequenced proximity linked DNA molecules that include a sequence that is mapped to the genomic region of interest;

assigning (101) an observed proximity score to each of a plurality of genomic fragments of a genome, the observed proximity score of each genomic fragment being indicative of a presence in the dataset of at least one sequencing read in proximity to the genomic region of interest and comprising a sequence corresponding to the genomic fragment;

assigning (102) an expected proximity score to each of at least one genomic fragment of the plurality of genomic fragments, based on the observed proximity scores of the plurality of genomic fragments, wherein the expected proximity score comprises an expected value of the proximity score of the at least one of the plurality of genomic fragments;

generating (103) an indication of a likelihood that said at least one genomic fragment of the plurality of genomic fragments is involved in a chromosomal rearrangement, based on the observed proximity score of said at least one genomic fragment of the plurality of genomic fragments and the expected proximity score of said at least one genomic fragment of the plurality of genomic fragments and identifying said genomic fragment as a candidate rearrangement partner. Preferred embodiments of this method are described further herein and FIG. 6 provides a particularly preferred embodiment of this method.

Once candidate rearrangement partners have been identified, the method comprising determining whether genomic fragments of the candidate rearrangement partner that are in proximity to said genomic fragments comprising sequences flanking the 5′ end of the genomic region of interest and genomic fragments of the candidate rearrangement partner that are in proximity to said genomic fragments comprising sequences flanking the 3′ end of the genomic region of interest are overlapping or linearly separated.

Genomic fragments in proximity to a first part of genomic region of interest or region flanking the region of interest will show either an “intermingled” or “divided” accumulation with genomic fragments in proximity to a second part of genomic region of interest or region flanking the region of interest. Fragments demonstrating intermingled accumulation are referred to herein as “overlapping” and fragments demonstrating divided accumulation are referred to as “linearly separated”. Preferably, the methods comprise determining whether genomic fragments of a candidate rearrangement partner in proximity to a first part of genomic region of interest or region flanking the region of interest and genomic fragments of a candidate rearrangement partner in proximity to a second part of genomic region of interest or region flanking the region of interest are, when mapped to a reference sequence of the candidate rearrangement partner, overlapping or linearly separated.

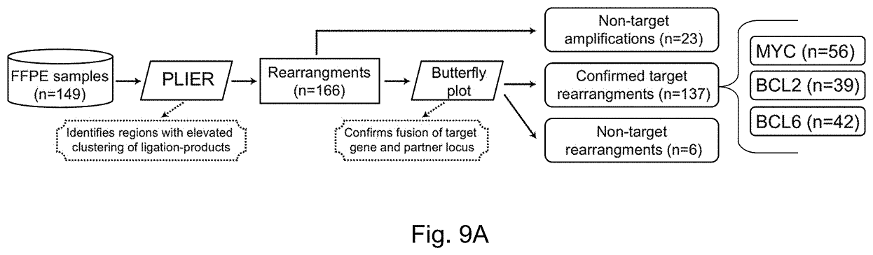

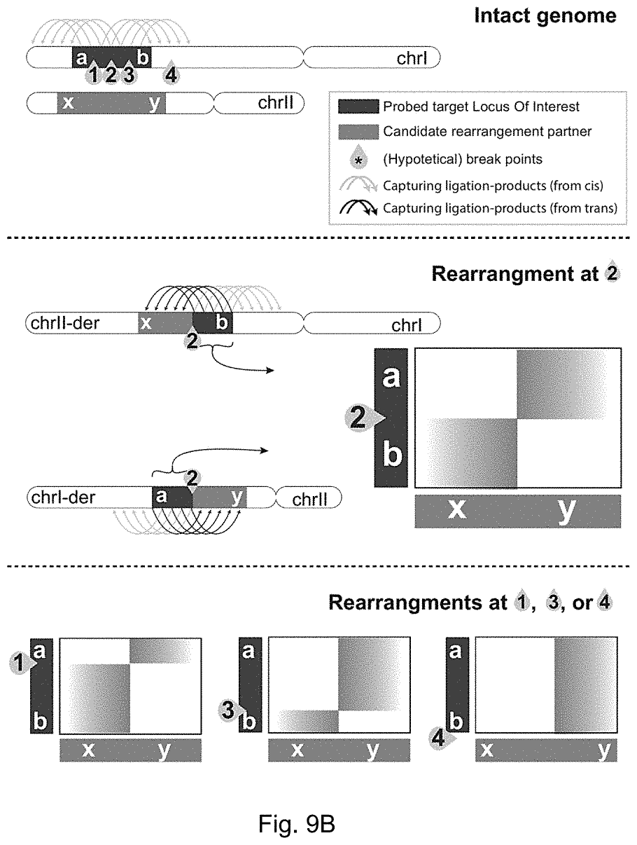

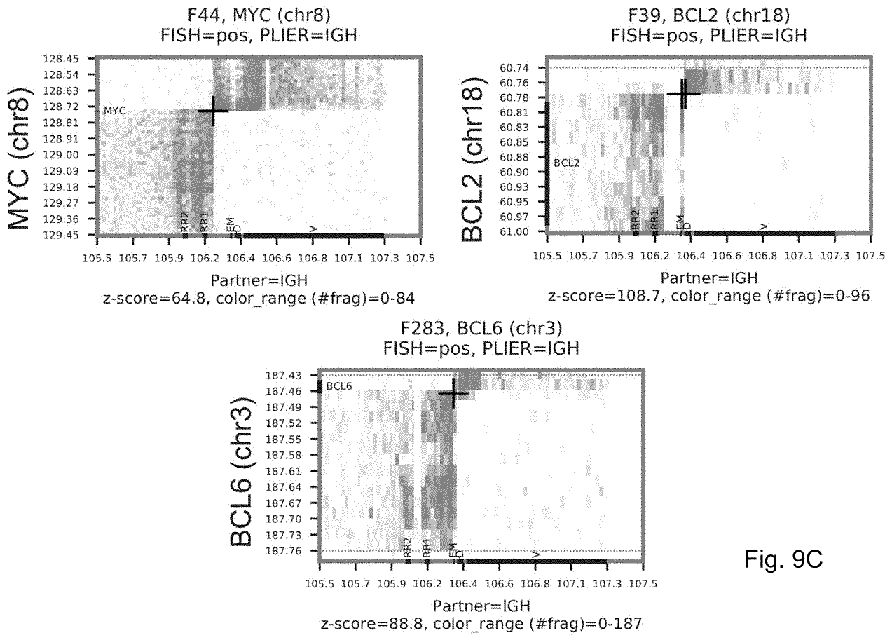

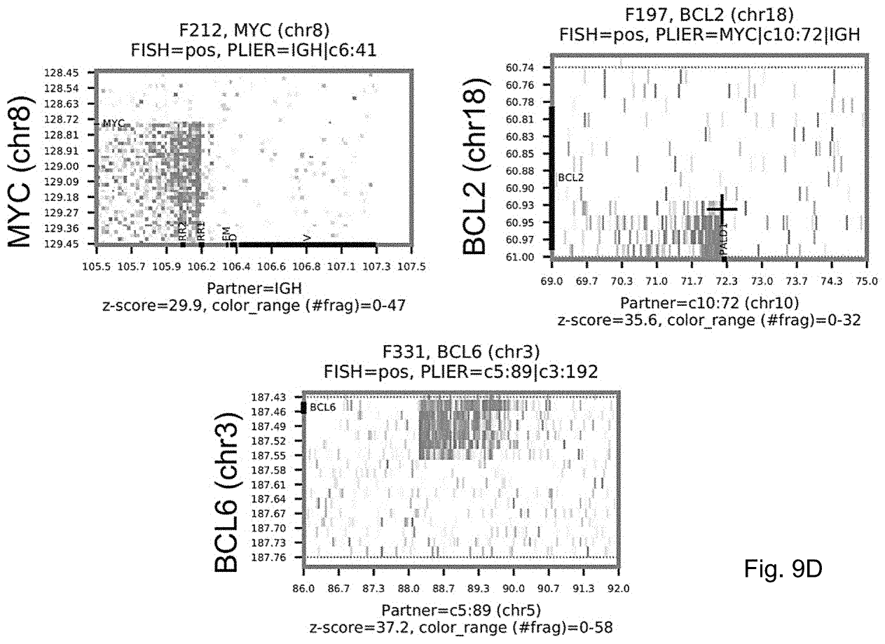

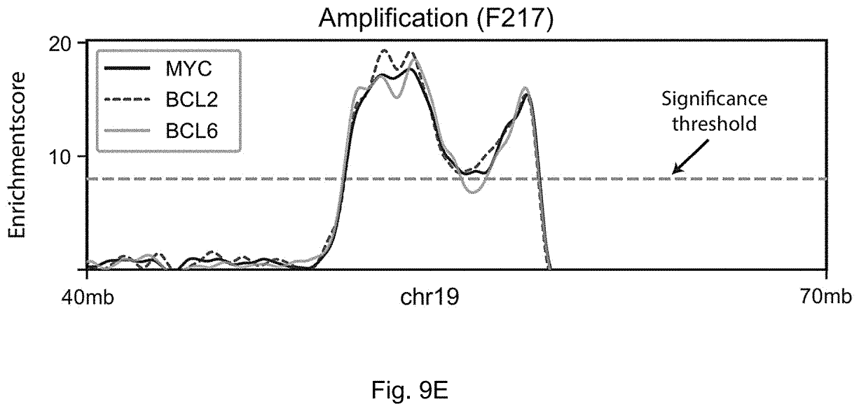

For example, the proximity products originating from the upstream and downstream sequences flanking the genomic region of interest can be analyzed to determine the distribution across the rearrangement partner. If the flanking genomic sequences show an overlapping (intermingled) accumulation of linked products on the linear reference template of the rearrangement partner, this indicates that the breakpoint is not located inside the genomic region of interest. If the flanking genomic sequences on the linear reference template of the rearrangement partner show a divided accumulation (also referred to herein as a “transition” or “linearly separated”), this indicates that the breakpoint is located inside the genomic region of interest. In regards to the rearrangement partner, the chromosomal breakpoint is positioned at the genomic segment that marks the transition of accumulation from proximity products originating from the upstream sequences flanking the genomic region of interest to proximity products originating from the downstream sequences flanking the genomic region of interest. If only one of the flanking regions (i.e., only 5′ flanking sequences or only 3′flanking sequences) contributes proximity products to the rearrangement partner, this indicates an unbalanced chromosomal rearrangement or a complex chromosomal rearrangement having a breakpoint inside the genomic region of interest and either a deletion of the other flanking sequences or its fusion to another partner in the genome (see, e.g., FIG. 9), as well as the insertion of foreign DNA.

In a preferred embodiment, the sequence location of genomic fragments (e.g., corresponding to a candidate rearrangement partner) in proximity to genomic fragments comprising sequences flanking the 3′ end of the genomic region of interest is compared to the sequence location of genomic fragments (e.g., corresponding to a candidate rearrangement partner) in proximity to genomic fragments comprising sequences flanking the 5′ end of the genomic region of interest. Linear separation of said candidate rearrangement partner genomic fragments is indicative of a chromosomal breakpoint junction within the genomic region of interest. In some embodiments, the method comprises analyzing whether enriched proximity linked products formed between the rearrangement partner and the targeted 5′ and 3′ sequences flanking the gene of interest, respectively, are separated on the linear chromosome template containing the rearrangement partner. Such linear separation is evidence for a chromosomal breakpoint inside the gene of interest.

One way to visualize overlapping and linear separation is to generate a matrix from sequence reads corresponding to genomic fragments where one axis represents the sequence location of genomic fragments corresponding to the genomic region of interest or sequences flanking the genomic region of interest and the other axis represents the sequence location of genomic fragments linked to the genomic region of interest or sequences flanking the genomic region of interest (e.g. a candidate rearrangement partner). The linked proximity products can be superimposed over the matrix such that each element within the matrix represents the number of times a linked product is found that comprises the corresponding genomic segment within or flanking the region of interest and a genomic segment linked to said corresponding genomic segment within or flanking the region of interest. See, e.g., FIG. 9B depicting rearrangement at position 4. The sequence of the candidate rearrangement partner overlaps at both positions “a” and “b” of the genomic region of interest. As is clear to a skilled person, overlapping candidate rearrangement partner sequences does not require that proximity ligated molecules comprising “a” and proximity ligated molecules comprising “b” must also include identical or physically overlapping rearrangement partner sequences. Rather a skilled person understands that there is an intermingling of such sequences. Compare this to linear separation described below.

As described above, one way to visualize linear separation is to generate a matrix. Linear separation is indicated if one or more coordinates on the axis representing the sequence location of the genomic region of interest and/or region flanking the genomic region of interest shows a transition in proximity frequency of the genomic segments from the candidate rearrangement partner. In particular, the proximity frequency of genomic segments from the candidate rearrangement partner in proximity to genomic fragments from the genomic region of interest and/or region flanking the genomic region of interest, which were enriched using the proximity assay disclosed herein, are compared.

In some embodiments, proximity linked products comprising the genomic region of interest are also enriched. Preferably, probes/primers are used to cover a significant portion of the genomic region of interest, such that proximity data is available for a significant portion of the genomic region of interest. If the matrix can be divided into four quadrants at a particular position based on maximal differences in frequencies between adjacent quadrants and minimal differences in frequencies within a quadrant, it indicates linear separation, which denotes a chromosomal break point. See, e.g., FIG. 9B depicting rearrangement at positions 1, 2, and 3 as well as the examples in FIG. 9C. These examples depict a likely reciprocal rearrangement.

Linear separation is also present when genomic fragments (e.g. corresponding to a candidate rearrangement partner) are in proximity to, e.g., sequences flanking the 5′region of the genomic region of interest but not to sequences flanking the 3′ genomic region of interest (or vice-versa). This form of linear separation can be visualized in a matrix by identifying one or more coordinates on the axis representing the sequence location of the genomic region of interest and/or region flanking the genomic region of interest that shows a transition in proximity frequency of the genomic segments from the candidate rearrangement partner. In the case of a non-reciprocal rearrangement, the transition is from a particular proximity frequency of the genomic segments from the candidate rearrangement partner to a (statistically significant) absence of candidate rearrangement partner sequences. In an exemplary embodiment, this form of linear separation can be visualized in a butterfly plot matrix by the presence of genomic fragments (e.g., corresponding to a candidate rearrangement partner) in a single quadrant and the (statistically significant) absence of candidate rearrangement partner sequences in the other three quadrants. See, e.g., the examples depicted in FIG. 9D.

In some embodiments, the method comprises assigning a score to the degree of intermingling (i.e., overlapping) of the proximity linked products. In some embodiments, the assigned score indicates whether the rearrangement is a reciprocal or non-reciprocal chromosomal rearrangement.

As demonstrated in the examples, enriching for proximity linked products that comprise genomic fragments comprising sequences flanking the 5′ end of the genomic region of interest and proximity linked products that comprise genomic fragments comprising sequences flanking the 3′ end of the genomic region of interest surprisingly allows for the confirmation of rearrangements resulting in breakpoint junctions within the genomic region of interest and reduces “false positives” (see FIG. 9A).

As described above, the methods may further comprise enriching for proximity linked products that comprise i) at least part of the genomic region of interest and ii) genomic fragments being in proximity to the genomic region of interest. In some embodiments, the method comprises providing a plurality of probes or primers that is at least partly complementary to the genomic region of interest. Each of the plurality of oligonucleotide probes/primers may be directed to a different or overlapping subsequence of the genomic region of interest. In some embodiments, the panel of probes/primers is designed to target the genomic region at intervals of at least one probe/primer every 100 kb, every 10 kb, or every 1 kb. Such methods are useful for determining the position of the chromosomal breakpoint junction fusing the candidate rearrangement partner to a position within the genomic region of interest, or rather for “fine-mapping” the breakpoint junction.

In such embodiments the methods further comprise sequencing said proximity linked DNA molecules comprising i) at least part of the genomic region of interest and ii) genomic fragments being in proximity to the genomic region of interest to produce genomic region of interest sequencing reads.

The methods may further comprise mapping the chromosomal breakpoint, wherein the mapping comprises detecting proximity ligated DNA molecules comprising at least part of the genomic region of interest and having linear separation of the rearrangement partner sequences. As is clear to a skilled person, the methods may include identifying proximity ligated molecules comprising genomic region of interest fragments that are closest in linear sequence to one another and having linear separation of the rearrangement partner sequences. This can be done by, for example, organizing proximity linked products (comprising at least part of the genomic region of interest and genomic fragments being in proximity to the genomic region of interest, e.g., a candidate rearrangement partner) according to their position of origin on the linear template of the genomic region of interest and analyzing, by means of for example sliding window approaches, how linear organization on the genomic region of interest is related to the linear location of their proximity linked products mapped to the rearrangement partner. The location that upon sliding across the genomic region of interest marks the transition from proximity linked products intermingling (i.e., overlapping) on the linear template of the rearrangement partner, to proximity linked products separated on the linear template of the rearrangement partner, demarcates the chromosomal breakpoint position inside the genomic region of interest.

In some embodiments, the mapping of the chromosomal breakpoint comprises generating a matrix for at least a subset of the sequencing reads, wherein one axis of the matrix represents the sequence location of the genomic region of interest and/or sequences flanking the genomic region of interest and the other axis represent the sequence location of the candidate rearrangement partner, wherein the matrix is generated by superimposing the sequencing reads over the matrix such that each element within the matrix represents the frequency of a proximity linked DNA molecule that comprises a genomic fragment of the genomic region of interest and a genomic fragment from the rearrangement partner. A preferred matrix is a butterfly plot. See, FIG. 9 for the mapping of breakpoint junctions in the BCL2 and MYC genes.

In some embodiments, the method comprising determining the sequence of the genomic region spanning the breakpoint, said method comprising identifying proximity ligated DNA molecules comprising i) breakpoint-proximal sequences of the genomic region of interest and ii) rearrangement partner sequences. One advantage of the methods described herein relates to the ability to filter ‘real’ fusion reads from ‘noise’ reads present in the sequencing data. Standard next-generation sequencing methods allow filtering steps primarily on differences in frequency (between real and noise) and/or prior knowledge on fusion partners. In some aspects of the disclosure, ‘real’ fusion reads can be separated from noise by first applying the PLIER algorithm that locates candidate rearrangement partners. Alternatively, or in addition to the PLIER algorithm, methods are provided using a plurality of probes/primer in order to further fine-map the location of the breakpoint. The creation of a matrix, such as a butterfly plot, assists in identifying the position of the breakpoint. The disclosed methods thus identify the proximity ligated molecules with the highest likelihood of comprising the genomic sequence comprising the breakpoint junction. This greatly reduces the background noise level. The identification of real fusion reads is also improved by discarding proximity ligated products that are fused at a restriction enzyme recognition site in the genome (+/−1 base pair), or rather at the restriction site used for fragmenting during the proximity ligation assay.

In some embodiments, the method further comprises determining the mutation (or rather sequence of a mutation) resulting from the chromosomal rearrangement.

The disclosure further provides a computer program product for detecting a chromosomal breakpoint fusing a rearrangement partner to a position within a genomic region of interest, said computer program product comprising computer-readable instructions that, when executed by a processor system, cause the processor system to:

generate a matrix for at least a subset of sequencing reads, wherein the sequencing reads correspond to the sequences of proximity linked products, said products comprising genomic fragments from the genomic region of interest or flanking the region of interest and wherein at least a subset of proximity linked products comprises a genomic fragment of a candidate rearrangement partner,

wherein one axis of the matrix represents the sequence location of the genomic region of interest and/or region flanking the genomic region of interest and the other axis represent the sequence location of the candidate rearrangement partner, wherein the matrix is generated by superimposing the sequencing reads over the matrix such that each element within the matrix represents the frequency of a proximity linked product that comprises a genomic segment of the genomic region of interest or flanking the region of interest and a genomic segment from the rearrangement partner, and

search the matrix to detect one or more coordinates on the axis representing the sequence location of the genomic region of interest and/or region flanking the genomic region of interest that shows a transition in proximity frequency of the genomic segments from the candidate rearrangement partner.

In some embodiments, the processor system searches the matrix to detect one or more elements that divides at least a part of the matrix into four quadrants, such that the differences in frequency between adjacent quadrants is maximized and the differences between opposing quadrants is minimized. Such embodiments are particularly useful in embodiments that also enrich for a plurality of proximity linked products that comprise different parts of the genomic region of interest. In some embodiments of the computer program product, the processor system compares the four quadrants identified and classifies the chromosomal breakpoint as resulting in a reciprocal rearrangement when two opposing quadrants exhibit minimal difference in frequency and the adjacent quadrants exhibit maximal differences in frequency or classifies the chromosomal breakpoint as resulting in a non-reciprocal rearrangement when a single quadrant exhibits the maximal difference in frequency compared to the other three quadrants. The computer program products described herein are useful for performing the methods as described herein.

In some embodiments, a computational method is used in the computer program product of methods described herein to automatically detect the breakpoint position. Standard template matching strategies in computer vision field (such as Kernel Search) are used to estimate the most likely position for splitting the matrix. In addition, by exploiting permutation strategies (i.e. shuffling ligation products across the matrix), the computation method estimates the significance of the detected pattern to reduce the error-rate of detected patterns. This approach is further enhanced if the computational method combines permutation strategies with smoothing strategies (such as Gaussian kernels) as well as scale-space modeling to reduce the intrinsic noise of pattern matching and significance estimation specially using a matrix that is often sparsely populated with observed proximity linked products.

### REFERENCES

Allahyar, A., Vermeulen, C., Bouwman, B. A. M., Krijger, P. H. L., Verstegen, M. J. A. M., Geeven, G., van Kranenburg, M., Pieterse, M., Strayer, R., Haarhuis, J. H. I., et al. (2018) Enhancer hubs and loop collisions identified from single-allele topologies. Nat. Genet. 50, 1151-1160.
Brant L, Georgomanolis T, Nikolic M, Brackley C A, Kolovos P, van Ijcken W, Grosveld F G, Marenduzzo D, Papantonis A. Exploiting native forces to capture chromosome conformation in mammalian cell nuclei. Mol Syst Biol. 2016 Dec. 9; 12(12):891.
Cairns, J., Freire-Pritchett, P., Wingett, S. W., Várnai, C., Dimond, A., Plagnol, V., Zerbino, D., Schoenfelder, S., Javierre, B. M., Osborne, C., et al. (2016). CHiCAGO: Robust detection of DNA looping interactions in Capture Hi-C data. Genome Biol. June 15; 17(1):127
Chesi, A., Wagley, Y., Johnson, M. E., Manduchi, E., Su, C., Lu, S., Leonard, M. E., Hodge, K. M., Pippin, J. A., Hankenson, K. D., et al. (2019). Genome-scale Capture C promoter interactions implicate effector genes at GWAS loci for bone mineral density. Nat. Commun. 10, 1260.
Choy, M. K., Javierre, B. M., Williams, S. G., Baross, S. L., Liu, Y., Wingett, S. W., Akbarov, A., Wallace, C., Freire-Pritchett, P., Rugg-Gunn, P. J., et al. (2018b). Promoter interactome of human embryonic stem cell-derived cardiomyocytes connects GWAS regions to cardiac gene networks. Nat. Commun. June 28; 9(1):2526.
Dao, L. T. M., Galindo-Albarrán, A. O., Castro-Mondragon, J. A., Andrieu-Soler, C., Medina-Rivera, A., Souaid, C., Charbonnier, G., Griffon, A., Vanhille, L., Stephen, T., et al. (2017). Genome-wide characterization of mammalian promoters with distal enhancer functions. Nat. Genet. 49, 1073-1081.
Dekker, J., Rippe, K., Dekker, M., and Kleckner, N. (2002) Capturing chromosome conformation. Science. 295, 1306-1311
Denker A, de Laat W. (2016). The second decade of 3C technologies: detailed insights into nuclear organization. Genes Dev. 30:1357-82.
de Vree P J P, de Wit E, Yilmaz M, van de Heijning M, Klous P, Verstegen M J A M, Wan Y, Teunissen H, Krijger P H L, Geeven G, Eijk P P, Sie D, Ylstra B, Hulsman L O M, van Dooren M F, van Zutven L J C M, van den Ouweland A, Verbeek S, van Dijk K W, Cornelissen M, Das A T, Berkhout B, Sikkema-Raddatz B, van den Berg E, van der Vlies P, Weening D, den Dunnen J T, Matusiak M, Lamkanfi M, Ligtenberg M J L, ter Brugge P, Jonkers J, Foekens J A, Martens J W, van der Luijt R, Ploos van Amstel H K, van Min M, Splinter E, de Laat W (2014). Targeted sequencing by proximity ligation for comprehensive variant detection and local haplotyping. Nature Biotechnology. October; 32(10):1019-25.
Dryden, N. H., Broome, L. R., Dudbridge, F., Johnson, N., Orr, N., Schoenfelder, S., . . . & Assiotis, I. (2014). Unbiased analysis of potential targets of breast cancer susceptibility loci by Capture Hi-C. Genome research, 24(11), 1854-1868.
Homminga, I., Pieters, R., Langerak, A. W., de Rooi, J. J., Stubbs, A., Verstegen, M., Vuerhard, M., Buijs-Gladdines, J., Kooi, C., Klous, P., van Vlierberghe, P., Ferrando, A. A., Cayuela, J. M., Verhaaf, B., Beverloo, H. B., Horstmann, M., de Haas, V., Wiekmeijer, A. S., Pike-Overzet, K., Staal, F. J., de Laat, W., Soulier, J., Sigaux, F., and Meijerink, J. P. (2011) Integrated transcript and genome analyses reveal NKX2-1 and MEF2C as potential oncogenes in T cell acute lymphoblastic leukemia. Cancer cell. 19, 484-497.
Hottentot Q P, van Min M, Splinter E, White S J. Targeted Locus Amplification and Next-Generation Sequencing. Methods Mol Biol. 2017; 1492:185-196.
Hughes, J. R., Roberts, N., McGowan, S., Hay, D., Giannoulatou, E., Lynch, M., De Gobbi, M., Taylor, S., Gibbons, R., and Higgs, D. R. (2014). Analysis of hundreds of cis-regulatory landscapes at high resolution in a single, high-throughput experiment. Nat. Genet. 46, 205-212.
Jäger, R., Migliorini, G., Henrion, M., Kandaswamy, R., Speedy, H. E., Heindl, A., Whiffin, N., Carnicer, M. J., Broome, L., Dryden, N., et al. (2015). Capture Hi-C identifies the chromatin interactome of colorectal cancer risk loci. Nat. Commun. February 19; 6:6178
Javierre, B. M., Sewitz, S., Cairns, J., Wingett, S M., Várnai, C., Thiecke, M. J., Freire-Pritchett, P., Spivakov, M., Fraser, P., Burren, O. S., et al. (2016). Lineage-Specific Genome Architecture Links Enhancers and Non-coding Disease Variants to Target Gene Promoters. Cell. November 17; 167(5): 1369-1384
Kwak, E. L., Bang, Y. J., Camidge, D. R., Shaw, A. T., Solomon, B., Maki, R. G., Ou, S. H., Dezube, B. J., Janne, P. A., Costa, D. B., Varella-Garcia, M., Kim, W. H., Lynch, T. J., Fidias, P., Stubbs, H., Engelman, J. A., Sequist, L. V., Tan, W., Gandhi, L., MinoKenudson, M., Wei, G. C., Shreeve, S. M., Ratain, M. J., Settleman, J., Christensen, J. G., Haber, D. A., Wilner, K., Salgia, R., Shapiro, G. I., Clark, J. W., and Iafrate, A. J. (2010) Anaplastic lymphoma kinase inhibition in non-small-cell lung cancer. The New England journal of medicine. 363, 1693-1703.
Li, G., Ruan, X., Auerbach, R. K., Sandhu, K. S., Zheng, M., Wang, P., Poh, H. M., Goh, Y., Lim, J., Zhang, J., et al. (2012). Extensive Promoter-Centered Chromatin Interactions Provide a Topological Basis for Transcription Regulation. Cell 148, 84-98.
Lieberman-Aiden, E., van Berkum, N. L., Williams, L., Imakaev, M., Ragoczy, T., Telling, A., Amit, I., Lajoie, B. R., Sabo, P. J., Dorschner, M. O., et al. (2009). Comprehensive mapping of long-range interactions reveals folding principles of the human genome. Science 326, 289-293.
Martin, P., McGovern, A., Orozco, G., Duffus, K., Yarwood, A., Schoenfelder, S., Cooper, N. J., Barton, A., Wallace, C., Fraser, P., et al. (2015). Capture Hi-C reveals novel candidate genes and complex long-range interactions with related autoimmune risk loci. Nat. Commun. November 30; 6:10069.
Mifsud, B., Tavares-Cadete, F., Young, A. N., Sugar, R., Schoenfelder, S., Ferreira, L., Wingett, S. W., Andrews, S., Grey, W., Ewels, P. A., et al. (2015). Mapping long-range promoter contacts in human cells with high-resolution capture Hi-C. Nat. Genet. 47, 598-606.
Montefiori, L. E., Sobreira, D. R., Sakabe, N. J., Aneas, I., Joslin, A. C., Hansen, G. T., Bozek, G., Moskowitz, I. P., McNally, E. M., and Nóbrega, M. A. (2018). A promoter interaction map for cardiovascular disease genetics. Elife. July 10; 7. pii: e35788
Mumbach, M. R., Satpathy, A. T., Boyle, E. A., Dai, C., Gowen, B. G., Cho, S. W., Nguyen, M. L., Rubin, A. J., Granja, J. M., Kazane, K. R., et al. (2017) Enhancer connectome in primary human cells identifies target genes of disease-associated DNA elements. Nat. Genet. 49, 1602-1612.
Orlando, G., Law, P. J., Cornish, A. J., Dobbins, S. E., Chubb, D., Broderick, P., Litchfield, K., Hariri, F., Pastinen, T., Osborne, C. S., et al. (2018). Promoter capture Hi-C-based identification of recurrent noncoding mutations in colorectal cancer. Nat. Genet. November; 49(11):1602-1612.
Pisapia, P., Lozano, M. D., Vigliar, E., Bellevicine, C., Pepe, F., Malapelle, U., and Troncone, G. (2017) ALK and ROS1 testing on lung cancer cytologic samples: Perspectives. Cancer. 125, 817-830.
Plenker, D., Riedel, M., Bragelmann, J., Dammert, M. A., Chauhan, R., Knowles, P. P., Lorenz, C., Keul, M., Buhrmann, M., Pagel, O., Tischler, V., Scheel, A. H., Schutte, D., Song, Y., Stark, J., Mrugalla, F., Alber, Y., Richters, A., Engel, J., Leenders, F., Heuckmann, J. M., Wolf, J., Diebold, J., Pall, G., Peifer, M., Aerts, M., Gevaert, K., Zahedi, R. P., Buettner, R., Shokat, K. M., McDonald, N. Q.,
Kast, S. M., Gautschi, O., Thomas, R. K., and Sos, M. L. (2017) Drugging the catalytically inactive state of RET kinase in RET-rearranged tumors. Sci Transl Med. 9, June 14; 9 (394).
Quinodoz, S. A. et al. Higher-Order Inter-chromosomal Hubs Shape 3D Genome Organization in the Nucleus. Cell 174, 744-757. e24 (2018).
Rao, S. S. P., Huntley, M. H., Durand, N. C., Stamenova, E. K., Bochkov, I. D., Robinson, J. T., Sanborn, A. L., Machol, I., Omer, A. D., Lander, E. S., et al. (2014). A 3D Map of the Human Genome at Kilobase Resolution Reveals Principles of Chromatin Looping. Cell 159, 1665-1680.
Schram, A. M., Chang, M. T., Jonsson, P., and Drilon, A. (2017) Fusions in solid tumours: diagnostic strategies, targeted therapy, and acquired resistance. Nature reviews. Clinical oncology. 14, 735-748
Schwartzman O, Mukamel Z, Oded-Elkayam N, Olivares-Chauvet P, Lubling Y, Landan G, Izraeli S, Tanay A. UMI-4C for quantitative and targeted chromosomal contact profiling. Nat Methods. 2016 August; 13(8):685-91.
Shaw, A. T., and Engelman, J. A. (2014) Ceritinib in ALK-rearranged non-small cell lung cancer. The New England journal of medicine. 370, 2537-2539.
Shaw, A. T., Ou, S. H., Bang, Y. J., Camidge, D. R., Solomon, B. J., Salgia, R., Riely, G. J., Varella-Garcia, M., Shapiro, G. I., Costa, D. B., Doebele, R. C., Le, L. P., Zheng, Z., Tan, W., Stephenson, P., Shreeve, S. M., Tye, L. M., Christensen, J. G., Wilner, K. D., Clark, J. W., and lafrate, A. J. (2014) Crizotinib in ROS1-rearranged non-small-cell lung cancer. The New England journal of medicine. 371, 1963-1971.
Simonis, M., Klous, P., Splinter, E., Moshkin, Y., Willemsen, R., de Wit, E., van Steensel, B., and de Laat, W. (2006). Nuclear organization of active and inactive chromatin domains uncovered by chromosome conformation capture-on-chip (4C). Nat. Genet. 38, 1348-1354.
van de Werken H, Landan G, Holwerda S, Hoichman M, Klous P, Chachik R, Splinter E, Valdes Quezada C, Öz Y, Bouwman B, Verstegen M, de Wit E, Tanay A, de Laat W. (2012). Robust 4C-seq data analysis to screen for regulatory DNA interactions. Nature Methods, 9:969-72.

The examples and embodiments described herein serve to illustrate rather than limit the invention. The person skilled in the art will be able to design alternative embodiments without departing from the spirit and scope of the present disclosure, as defined by the appended claims and their equivalents. Reference signs placed in parentheses in the claims shall not be interpreted to limit the scope of the claims. Items described as separate entities in the claims or the description may be implemented as a single hardware or software item combining the features of the items described.

### Examples

Structural variation (SV) in the genome is a recurring hallmark of cancer. Translocations (genomic rearrangements between chromosomes) in particular are found as recurrent drivers in many types of hematolymphoid malignancies. They are also increasingly appreciated in various types of solid tumors, such as lung- and prostate cancer and soft tissue sarcomas, serving as diagnostic, prognostic and even predictive parameters to guide treatment choice. Translocation analysis of specific sets of target genes is therefore increasingly implemented in routine diagnostic workflows for these malignancies. Diagnostic pathology practice is highly dependent on formalin-fixation and paraffin embedding (FFPE) procedures. The resulting FFPE specimen blocks provide a long-term preservation method and are particularly suitable for morphological assessment, including immunohistochemistry and in situ hybridization techniques (ISH). Currently, fluorescence in situ hybridization (FISH) is the “gold standard” for translocation detection in lymphoma FFPE samples. Although this method is commonly applied worldwide and successful in many instances, it has various limitations. FISH assessment is reliant on sufficient morphology. Therefore, crushing artifacts, poor fixation, extensive necrosis and apoptosis, that frequently impair morphology, often preclude reliable interpretation. Furthermore, even though FISH assays can be routinely performed in an automated fashion identical to immunohistochemistry, the analysis of the results and rearrangement detection is largely performed manually, which is labor intensive, error prone and expensive. Moreover, FISH assessment may be difficult, equivocal or subjective in case of uncommon breakpoints, polysomies or deletions that result in complex patterns of fluorescent signals^{1,2}. The routinely used break-apart FISH method fails to identify translocation partners, whereas fusion-FISH is only applicable in specific situations where the translocation partner is known, such as the MYC-IGH translocation. Knowing the exact composition of the rearrangement is imperative information that often delineates tumor progression behavior and its subclassification³. Finally, FISH analyses cannot be multiplexed.

More recently, Next-Generation Sequencing (NGS) DNA capture methods have been introduced for rearrangement detection in selected gene panels in FFPE samples, which makes it possible to detect breakpoints at base pair resolution and identify translocation partner genes⁴⁻⁷. However, such methods rely on capturing unambiguous fusion-reads, which can be challenging when non-unique sequences flank the breakpoint⁸. This is a common situation, especially in translocations in malignant lymphoma that typically involve immunoglobulin and T-cell receptor genes as translocation partners to oncogenes⁹. RNA-based detection methods are another approach for rearrangement detection in FFPE material and currently introduced in daily practice for those rearrangements that result in a chimeric or altered RNA product, as is typical for soft tissue tumors¹⁰⁻¹² RNA is less stable than DNA, which sometimes could affect performance of RNA-based diagnostic methods in FFPE specimens¹³. Furthermore, RNA-based detection methods cannot detect rearrangements in non-coding sequences that drive cancer through regulatory displacement effects. This is most often the case in malignant lymphoma, in which immunoglobulin- and T-cell receptor enhancer sequences mediate overexpression of further unaltered oncogenes. Taken together, there is still a clear need in daily diagnostic pathology practice for methodologies that more robustly detect and precisely characterize translocations in FFPE specimens.

Importantly, the formalin fixation and (unscheduled) DNA fragmentation in pathological tissue processing are obligatory steps in proximity-ligation (or ‘chromosome conformation capture’) methods. Originally invented to study chromosome folding¹⁴, proximity-ligation methods use formaldehyde-mediated fixation followed by in situ DNA fragmentation and ligation, to fuse DNA fragments that are most proximal within the cell nucleus. Then NGS and quantitative analyses of ligation products can provide a relative estimate for contact frequencies between pairs of sequences in the cell population and thereby enable the analysis of recurrent chromosome folding patterns. The most dominant factor that determines the contact frequency between a pair of DNA sequences is their linear adjacency on the same chromosome, whereby such contact frequency decays exponentially with increased linear separation between the two DNA sequences. Intriguingly, genomic rearrangements change the linear sequence of chromosomes and thereby alter DNA contact patterns that are generated in proximity-ligation methods. Based on this understanding, variants of proximity-ligation methods have been introduced as powerful technologies for the identification of genomic rearrangements¹⁵⁻²⁰. Proof-of-concept that proximity-ligation methods can also detect SVs in FFPE material was recently provided in a non-blind study that applied a Hi-C protocol (i.e. a genome-wide variant of proximity-ligation assays) to 15 FFPE tumor samples. In most cases, this method (called “Fix-C”) gave visually appreciable altered contact frequencies in genes previously scored to harbor rearrangement by FISH²¹. While potentially relevant to identify novel rearranged genes, such a genome-wide analysis requires expensive deep sequencing that is less relevant to clinical settings where the identification of rearrangements in selected genes with known clinical significance is required.

Here, we present FFPE-Targeted Locus Capture (FFPE-TLC), which uses in situ ligation of crosslinked DNA fragments, combined with oligonucleotide probe sets to selectively pull down, sequence and analyze the proximity-ligation products of genes with known clinical significance. FFPE-TLC was blindly applied to 149 lymphoma and control FFPE samples, obtained by resections or needle biopsies. Rearrangements were automatically scored using ‘PLIER’ (Proximity-Ligation based IdEntification of Rearrangements), a dedicated computational and statistical framework that processes FFPE-TLC sequenced datasets and identifies rearrangement partners of target genes based on their significantly enriched proximity-ligation products. Comparison of FISH, targeted NGS-capture and FFPE-TLC results shows that FFPE-TLC outperforms both methods in specificity, sensitivity and details provided on the detected rearrangements. Therefore, FFPE-TLC is a powerful new tool for SV detection in FFPE samples in malignant lymphoma and other translocation-mediated malignancies.

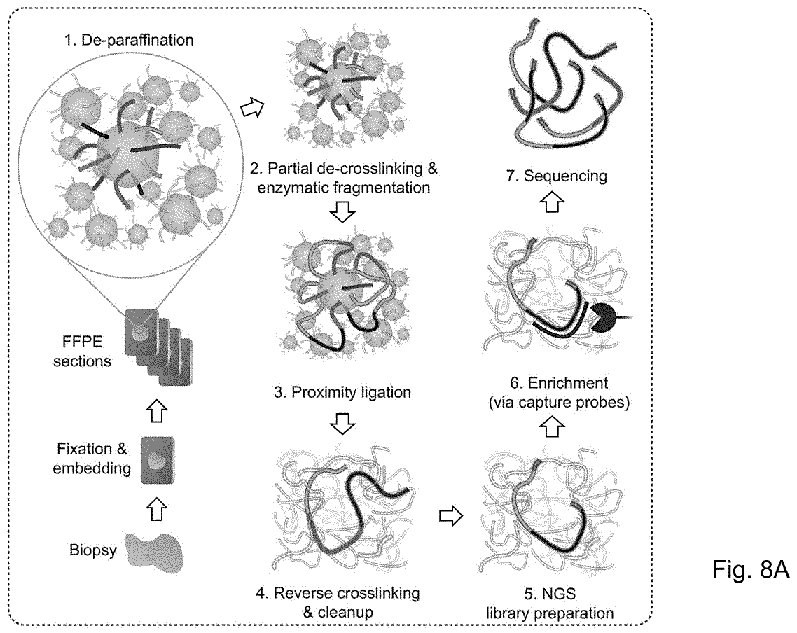

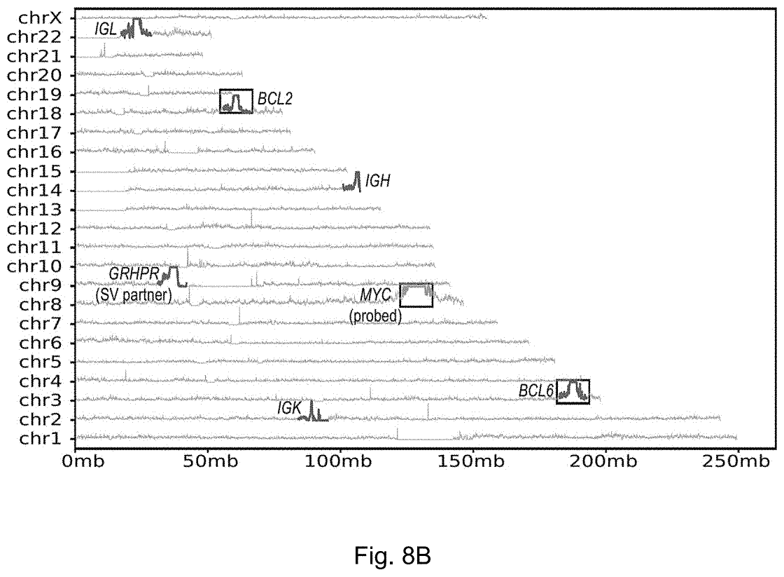

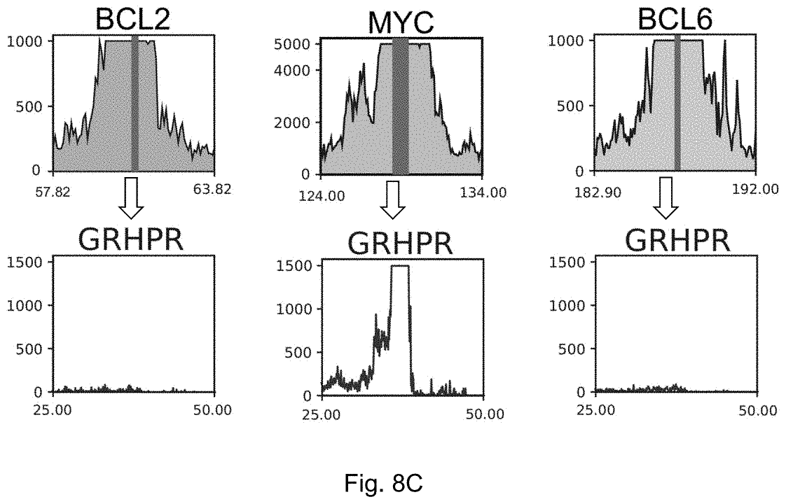

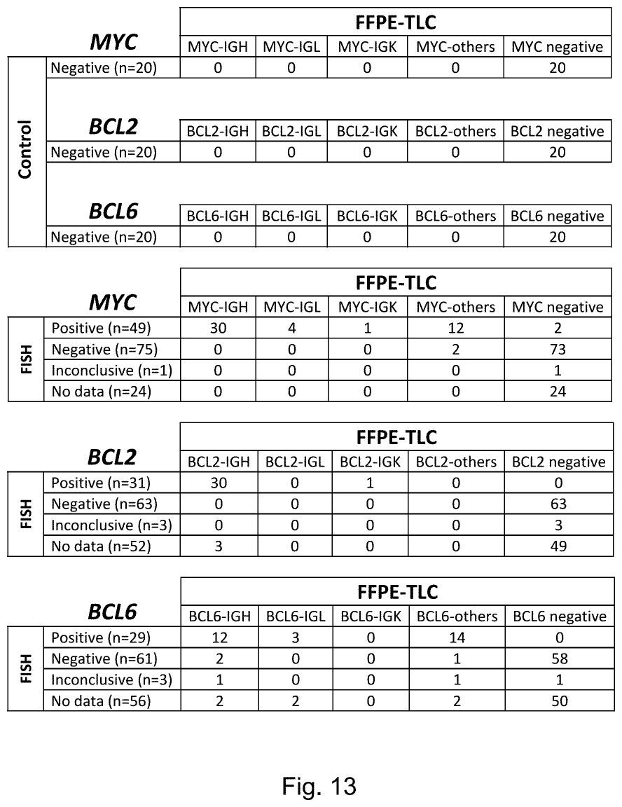

Briefly, in FFPE-TLC an FFPE scroll of a representative tumor sample is deparaffinized and mildly de-crosslinked to enable in situ DNA digestion by a restriction enzyme (NlaIII) that creates fragments with a median size of 141 bp. After in situ ligation and reverse crosslinking, standard protocols for (probe-based) hybridization capturing are followed (see Methods for details) and resulting libraries are sequenced in an Illumina sequencing machine (FIG. 8A and FIG. 13). In our current probe panel for lymphoma, we targeted the BCL2, BCL6, MYC genes and immunoglobulin loci IGH, IGK, IGL as well as other loci implicated in hematolymphoid malignancies. We applied FFPE-TLC to 129 lymphoma tumor samples selected for the presence or absence of rearrangements involving MYC, BCL2 or BCL6, as originally detected by FISH (FIG. 13). Additionally, 20 FFPE samples from reactive lymph nodes (mostly from breast cancer patients) were included that were not analyzed by FISH but were expected to be devoid of rearrangements in the six target genes. Samples were provided by five different medical centers in the Netherlands and differed in tissue block age, degree of DNA fragmentation and the presence of necrosis and/or crushing damage (data not shown). All 149 samples were anonymized and therefore, the presence or absence of rearrangements in any of the target genes were hidden from us in this (blind) study. To illustrate results, FIG. 8B shows a genome-wide coverage of sequences retrieved from a typical FFPE-TLC experiment. A closer inspection of sequences captured at and around the probe-targeted loci of MYC, BCL2 or BCL6 (FIG. 8C) highlights the added value of combining NGS capture with proximity-ligation for rearrangement detection: not only are the probe-complementary genomic sequences (in blue) retrieved efficiently by FFPE-TLC, it also strongly enriches mega bases of the flanking sequences (i.e. the proximity-ligation products, shown in FIG. 8C for MYC (pink), BCL2 (brown) and BCL6 (orange)). Since rearrangements with target loci juxtapose them to new flanking sequences, rearranged partner loci show an increased density of proximity-ligation sequences in FFPE-TLC and therefore can be uncovered. This phenomenon is depicted in FIG. 8B where MYC (in green) forms an unusually large number of proximity-ligation products with a locus containing the GRHPR gene (in red), indicative of tumor cells carrying this translocation²².

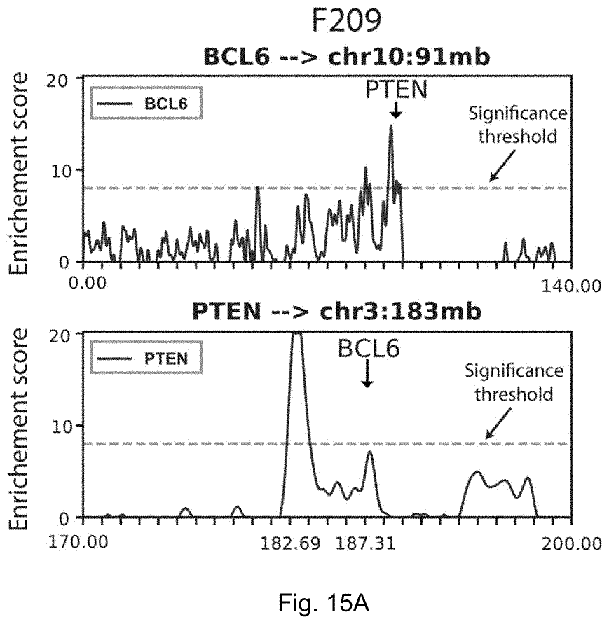

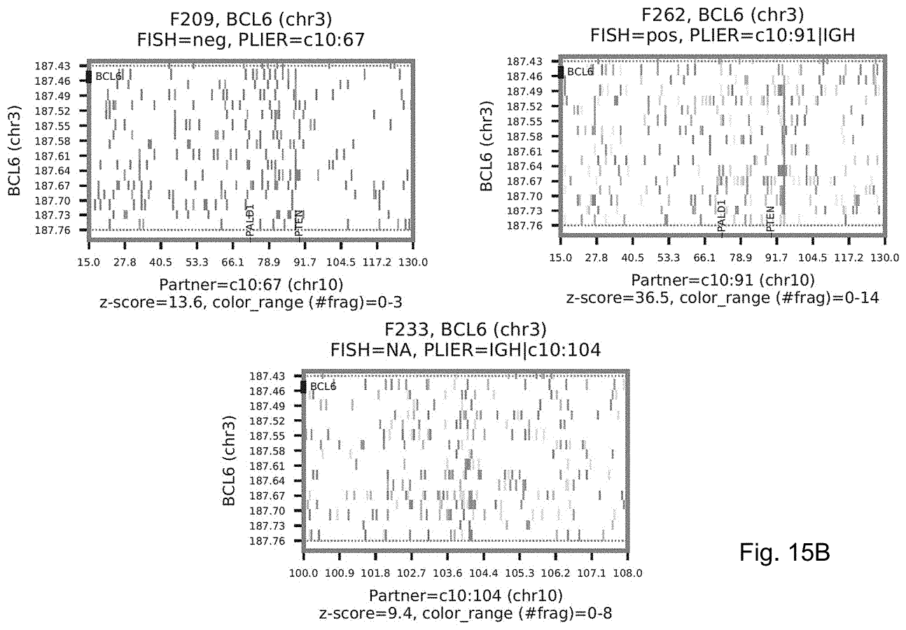

To objectively identify rearrangement partner genes in FFPE-TLC datasets in an automated fashion we developed a computational pipeline called PLIER (Proximity-Ligation based IdEntification of Rearrangements). In brief, PLIER initially demultiplexes sequenced FFPE-TLC samples into multiple FFPE-TLC datasets where each dataset consists of proximity-ligation products that are captured by a specific targeted gene (e.g. MYC). Then, for a given FFPE-TLC dataset (of a target gene), PLIER evaluates the density of proximity-ligation products across the genome to assign and compare an observed and expected proximity score to genomic intervals and calculate an enrichment score (see Methods and FIG. 15 for details). Genomic intervals with significantly elevated enrichment score are prime candidate rearrangement partners of the targeted gene. We initially identified the optimal parameters for PLIER through a comprehensive optimization procedure (see Methods for details on the optimization procedure). We then applied PLIER to all 149 samples to search for rearrangements involving the three clinically relevant targeted genes MYC, BCL2 and BCL6. An overview of the identified rearrangements and their comparison with FISH diagnostics is provided in FIG. 13. Across 20 control samples, FFPE-TLC detected no rearrangements, demonstrating the robust capability of PLIER in masking the intrinsic topological and methodological noise that inevitably is present in (FFPE) proximity-ligation datasets, while able to detect rearrangements involving MYC, BCL2 and BCL6 across the lymphoma samples.

In total, PLIER identified 137 rearrangements involving MYC, BCL2 and BLC6: 56 MYC rearrangements (in 49 lymphoma samples), 39 BCL2 rearrangements (in 34 samples) and 42 BCL6 rearrangements (in 40 samples) (FIG. 9A). To unambiguously assess whether PLIER-identified genomic regions were true rearrangements of the interrogated target genes, we closely inspected the distributions of their proximity-ligation products along the linear sequences of each presumed partner, in so-called butterfly plots²³. If engaged in a reciprocal translocation, each locus should reveal a “breakpoint” location separating its upstream sequences that preferentially form proximity-ligation products with one side of the partner locus, from its downstream sequences that preferentially contact and ligate the other part of the partner locus (FIG. 9B). FIG. 9C shows three examples of reciprocal rearrangements uncovered by butterfly plots, involving MYC, BCL2 and BCL6, respectively. Rearrangements can also be non-reciprocal, such that only one part of a target locus fuses to a given partner. FIG. 9D shows butterfly plots of these more complex rearrangements of MYC, BCL2 and BCL6. Across all analyzed samples, MYC was found to be involved in 41 reciprocal translocations (26 with IGH and 15 with non-IG loci) and 15 more complex rearrangements (4 with IGH), BCL2 in 34 reciprocal translocations (33 with IGH and 1 with IGK) and 5 more complex rearrangements, and BCL6 in 37 reciprocal translocations (16 with IGH, 5 with IGL and 16 with non-IG loci) and 5 more complex rearrangements.

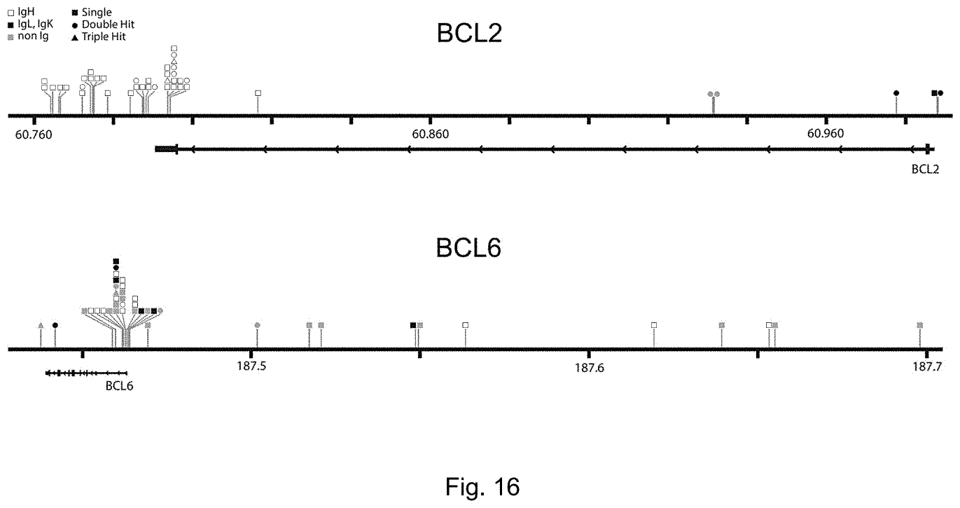

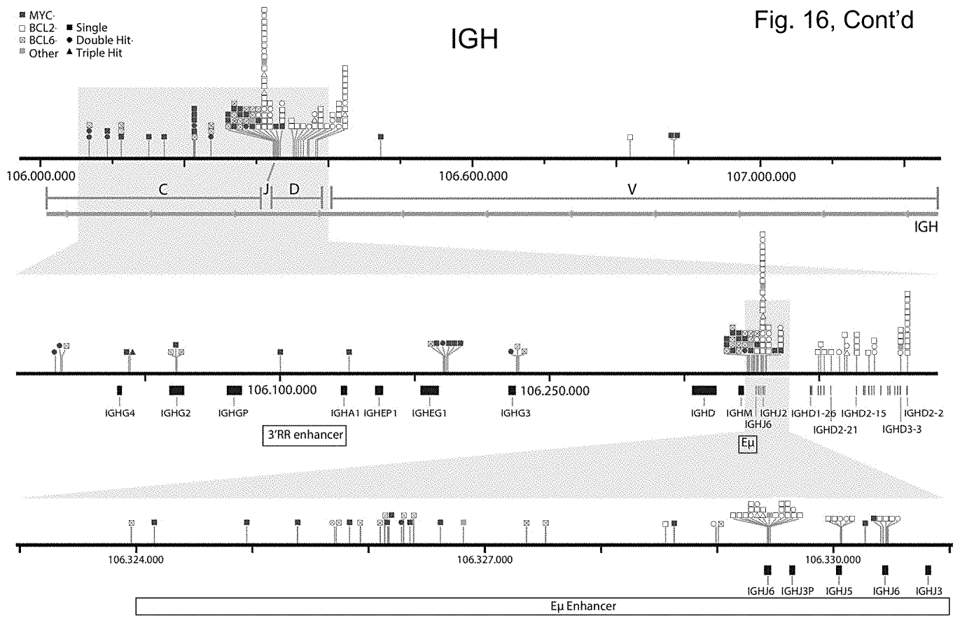

In addition to the 137 rearrangements with breakpoints in the MYC, BCL2 or BLC6 locus, PLIER was expected to also detect two bystander categories of genomic rearrangements that also can yield significant enrichment in proximity-ligation products. The first were amplified genomic regions (copy number variations); they could be distinguished from true positive rearrangements since PLIER scored them with all target genes (FIG. 9E). PLIER discovered 23 amplifications throughout the genome across all analyzed lymphoma samples. The second bystander category scored by PLIER were genomic rearrangements involving the chromosome that contained the target gene, but with breakpoints outside the probe-targeted region. As a consequence, such rearrangement showed no linear transition in proximity-ligation signals between the identified rearrangement and the target locus in butterfly plots (see FIG. 9B). Six of these rearrangements were found and for two cases (F209 and F262) we confirmed a rearrangement involving chromosome 3 but with a breakpoint mega bases away from the BCL6 locus (FIG. 16). Bystander rearrangements scored by PLIER were considered irrelevant for the gene of interest and were therefore classified as negative.

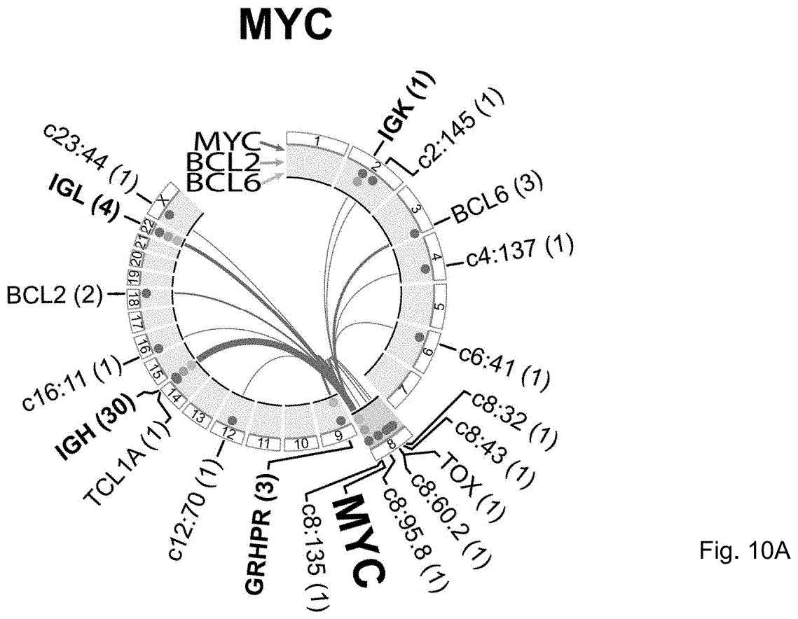

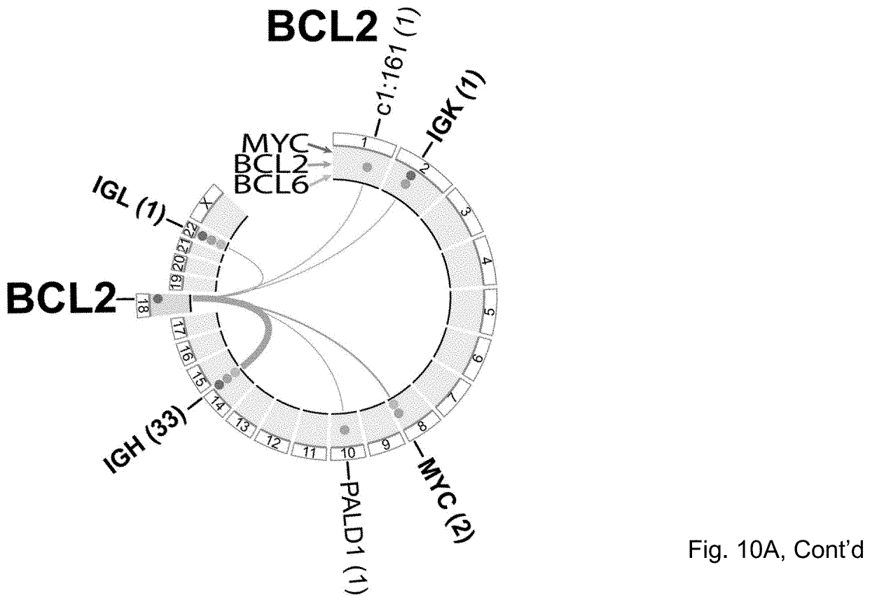

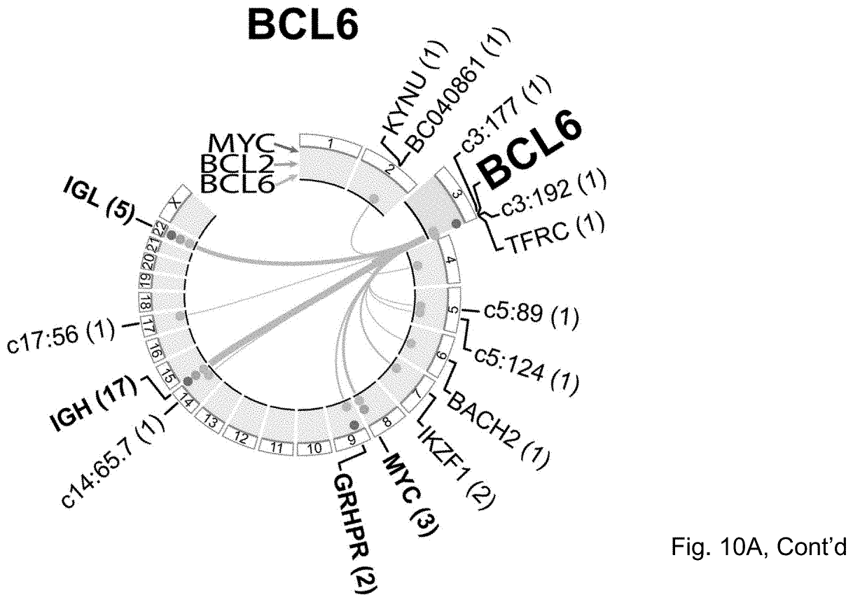

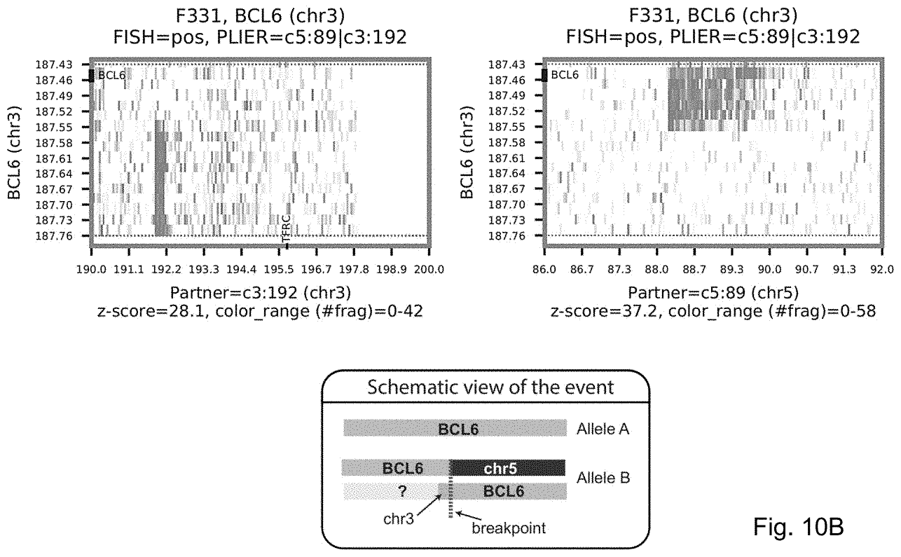

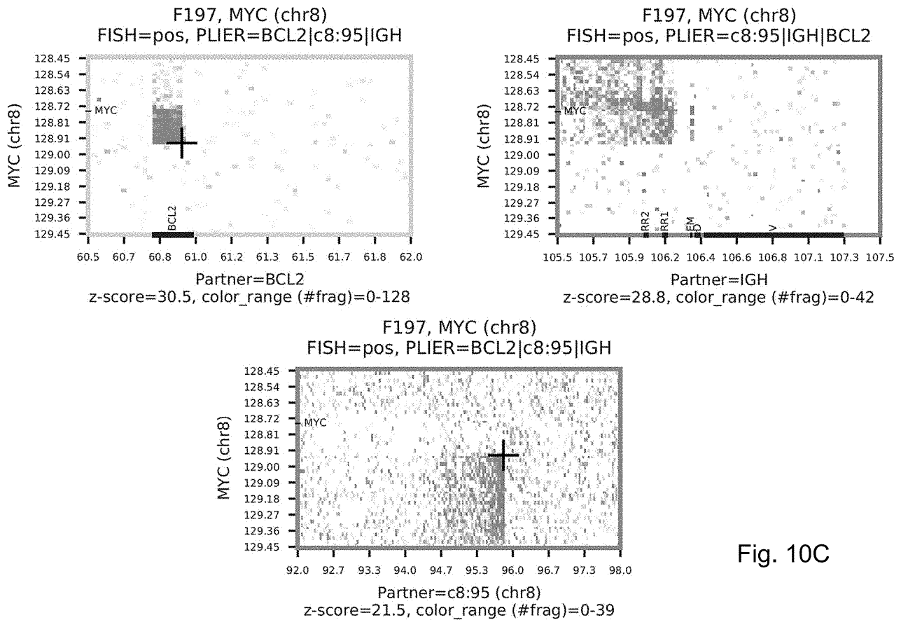

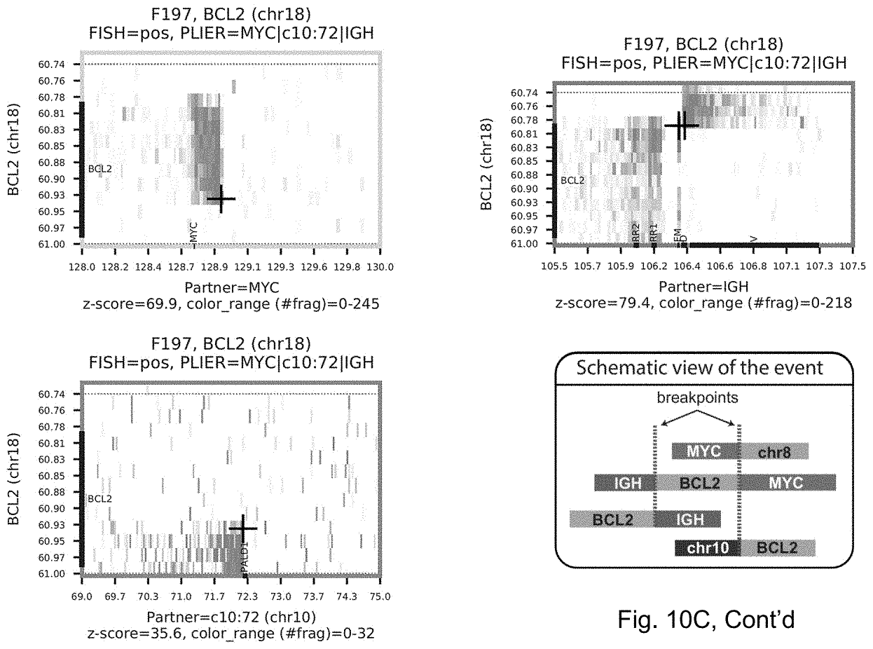

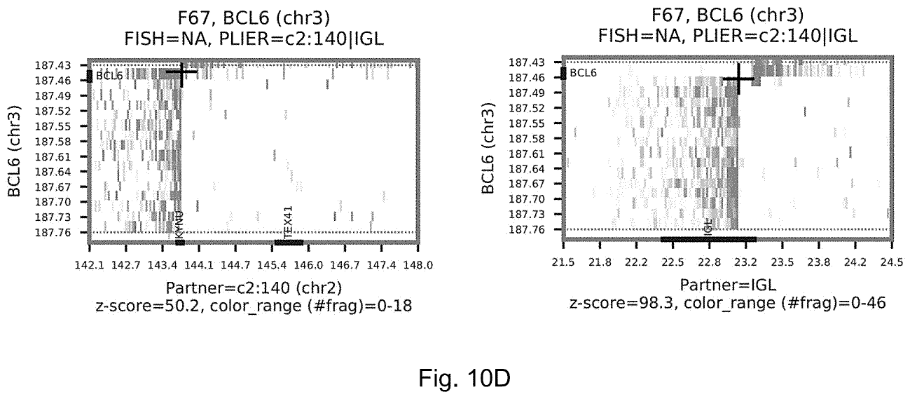

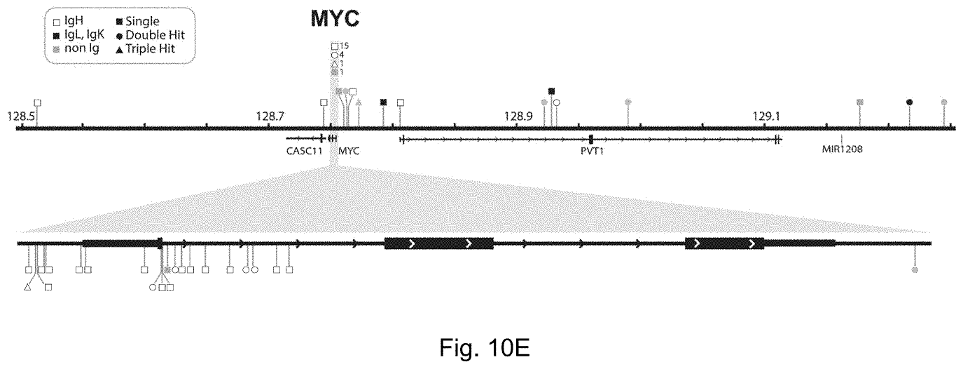

FIG. 10A provides a graphical overview of the rearrangement partners identified in this study using Circos plots²⁴. In our collection of samples, we found 3 samples positive for a translocation in MYC and BCL2 and BCL6 (i.e. triple hit), 19 samples positive for a translocation in both MYC and BCL2 or BCL6 (double hit), and 8 samples carrying a rearrangement in both BCL2 and BCL6. In 5 tumors, MYC was either directly fused to the BCL6 (F72, F190, F194) locus, or involved in a complex 3-way fusion with IGH and BLC2 (F197, F274). Apart from the immunoglobulin loci, we found several other recurrent rearrangement partners, including the KYNUITEX41 locus (F67, F188, with BCL6 and F201 with MYC), TBL1XR1 (F49, F273, F329, with BCL6), IKZF1 (F210, F281, with BCL6) and the TOX locus (F74, F271, with MYC). Strikingly, GRHPR was found 5 times as a rearrangement partner of BCL6 (F77, F199) and MYC (F202, F209, F269) (FIG. 10A). In cases such as F197 (MYC) and F331 (BCL6) we found strong indications for a non-reciprocal translocation event that fuses the different parts of the target locus to different genomic partners (FIG. 10B). In other instances, there was evidence for allelic three-way rearrangements, often involving the IGH locus, MYC (F50, F212, F274), BCL2 (F193, F274, F282) or BCL6 (F77) and a third partner (FIG. 10C, for examples). Further, in rare cases such as F67 (BCL6) (FIG. 10D), F202 (MYC) and F197 (BCL2) both alleles of the targeted locus independently appeared to be involved in rearrangements.

Using FFPE-TLC and PLIER, we were readily able to retrieve 90 breakpoint-spanning fusion-reads for the 137 identified SVs involving BCL2, BCL6 or MYC. Mapping the breakpoints to the target genes as well as to the IGH locus allowed inspection of recurrent breakpoint clusters in MYC, BLC2, BCL6 and IGH, as described previously^{5,25} (FIG. 10E and FIG. 15).

Even though probe design at IG loci was not optimal (as probes centered only on the enhancer regions), PLIER identified most (79 out of 91) rearrangements with MYC, BCL2 and BCL6 also reciprocally, when targeting the IG genes. Additionally, many rearrangements were found joining the IG loci with other genes, most of which have been described as rearrangement partners: IGH-PAX5/GRHPR (F21)^{22, 26} IGH-FOXP1 (F41)²⁷, IGH-PRDM6 (F43), IGH-CPT1A (F58)²⁸, IGL-BACH2 (F223)²⁹ and IGH-ACSF3 (F278)³⁰. Such cases warrant further investigation, particularly since they were found in samples not carrying other known drivers of lymphoma.

For validation and to explore an alternative proximity-ligation method, we processed 47 FFPE samples with 4C-seq³¹. In 4C-seq, inverse PCR instead of hybridization capture is used to enrich proximity-ligation products that are formed with selected sites of interest³². For this study, a multiplex 4C PCR was used with 14 primer sets distributed over the MYC, BCL2 and BCL6 locus and 7 primer sets targeting the IGH, IGL and IGK loci (total 21 primer sets). A modified version of PLIER was used to support the FFPE-4C type of data and score rearrangement partners (see Methods). Across all tested samples results were concordant between FFPE-TLC and FFPE-4C, with two exceptions (F54 and F67) where FFPE-4C failed to detect the rearrangement. Both were older samples, dating from 2007 and 2009, respectively, with severe DNA fragmentation. This suggested that FFPE-TLC is more tolerant to poor sample quality than FFPE-4C, which could be expected given that 4C additionally requires the circularization of (small) proximity-ligation products.

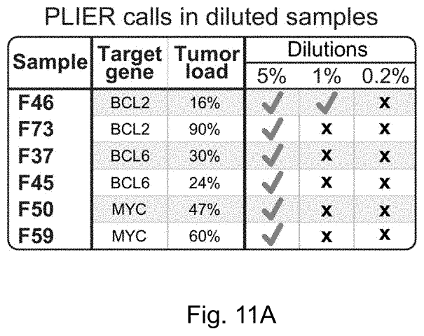

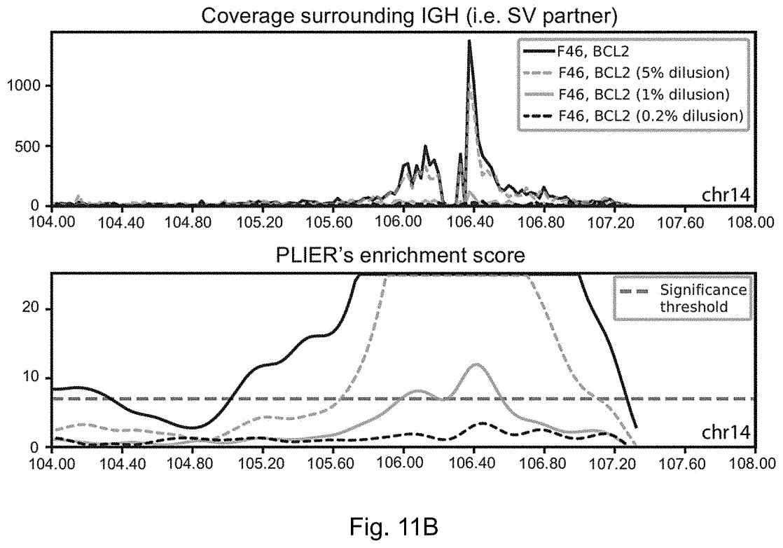

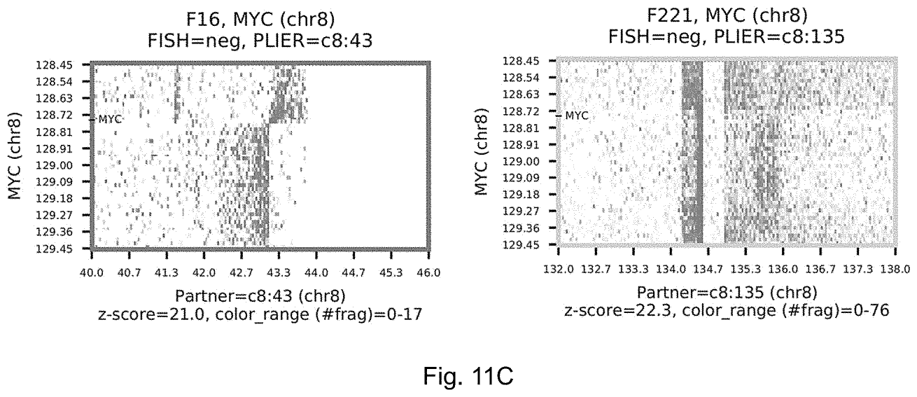

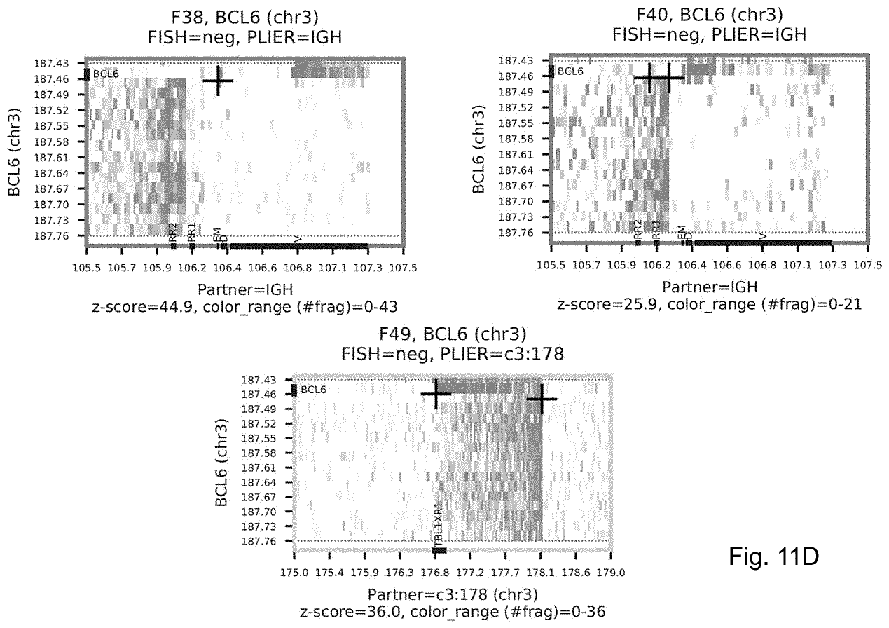

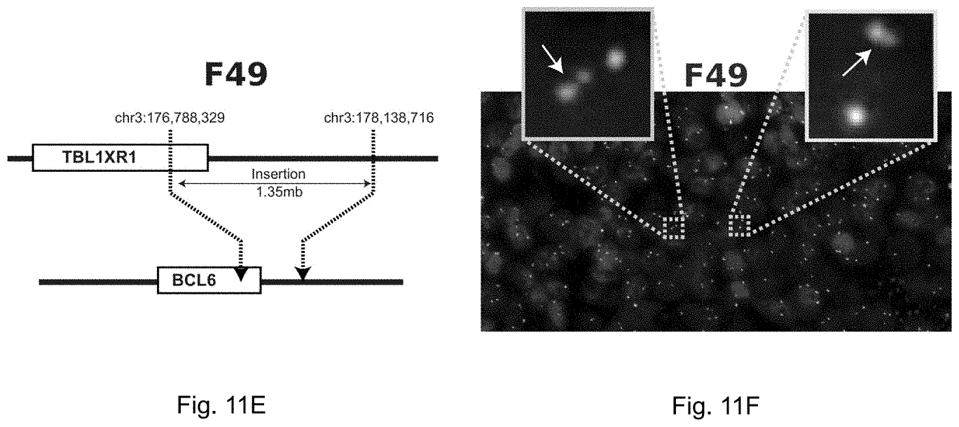

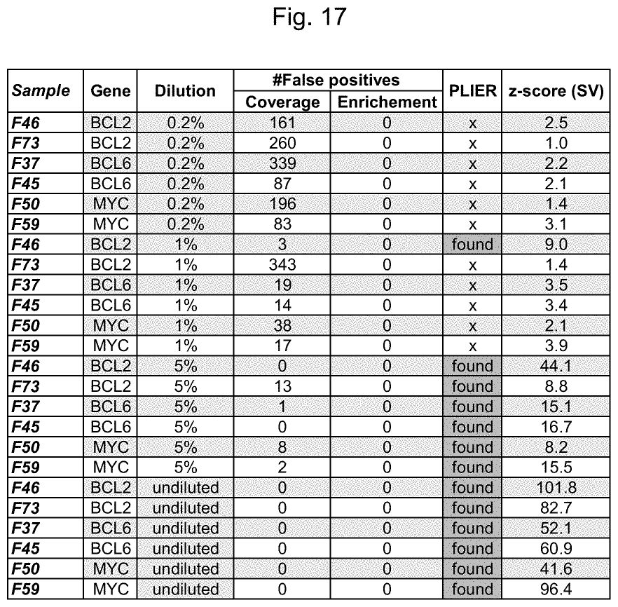

A major aim of our studies was to compare FFPE-TLC to FISH as a diagnostic method for rearrangement detection in FFPE specimens. Given background scoring results in negative control tissue, FISH is generally considered negative in diagnostic practice if aberrant signals occur in less than 10-20% of cells (the exact cut-off can differ per gene and per diagnostic center). The sensitivity of FFPE-TLC relies on PLIER's ability to identify candidate rearrangement partners. To more systematically investigate PLIER performance and sensitivity, we took six FFPE samples carrying FISH-validated rearrangements in MYC (2×), BCL2 (2×) and BCL6 (2×) with known percentages of FISH-positive cells, and diluted each sample (prior to probe pulldown) with control material not carrying the rearrangement, to percentages of 5%, 1% and 0.2%. We found that PLIER made no false-positive calls in any of the samples and confidently scored the actual rearrangement partner in all samples having 5% or more positive cells (see FIG. 11A-B and FIG. 17). This suggested that FFPE-TLC offers superior sensitivity as compared to FISH. However, the clinical implications of low translocation percentages caused by low tumor cell percentage or by tumor heterogeneity needs to be determined.

We compared the original FISH results to our FFPE-TLC results. Out of the 49 samples scored MYC positive by FFPE-TLC, 47 samples were also classified as such by FISH (FIG. 13). The MYC rearrangements missed by FISH were both in cis, with partners on the same chromosome 8 (F16 and F221: here FISH detected multiple signals) (FIG. 11C). For BCL2, 31 out of the 34 samples that we scored positive had also previously been reported by FISH: the three newly identified rearrangements, each carrying a BCL2-IGH translocation, had not been analyzed by FISH. For BCL6, 29 out of the 40 tumors with a BCL6 rearrangement had also been scored as such by FISH. Three BCL6 rearrangements (F38, F40, F49) were not detected by FISH (FIG. 11D), in two of instances because of below threshold percentages of cells with a rearrangement (10% (F38) and 6% (F40)). In the third case (F49), FFPE-TLC detected a 1.35 Mb insertion of the TBL1XR1 locus into the BCL6 locus (FIG. 11E). With hindsight, some split of signals could be observed in the FISH image (FIG. 11F) that originally was considered irrelevant. Two FFPE-TLC identified BCL6 rearrangements (one of which with IGH) were previously considered inconclusive by FISH because of single fluorescent signals (F25, F261). Six newly identified BCL6 rearrangements (2×IGH, 2×IGL) had not been analyzed by FISH (FIG. 13). Vice versa, all rearrangements scored by FISH were confirmed by FFPE-TLC, except for two (F217 and F322, both described as having a complex karyotype). Whether FFPE-TLC or FISH was wrong here could not be determined, unfortunately. In summary, all 149 samples analyzed FFPE-TLC showed very high concordance with FISH. It missed two rearrangements apparently scored by FISH but also identified and characterized two MYC rearrangements and five BCL6 rearrangements that were not scored by FISH. Moreover, FFPE-TLC's capacity to analyze multiple genes in parallel for their involvement in rearrangements, enabled discovering 9 cases of BCL2 and BCL6 rearrangements in samples that had not been tested for these rearrangements by FISH. In four cases, this discovery changed the original tumor classification of the samples. Sample F16 was reclassified from “no hit” to “double-hit” (DH) for MYC and BCL2 rearrangements, sample F67 from single (MYC) hit to a MYC-BCL6 DH tumor (with partners IGH and IGL), sample F194 from single (MYC) hit to MYC-BCL2-BCL6 triple hit (TH, although MYC and BCL6 fused together) and sample F209 from DH to TH.

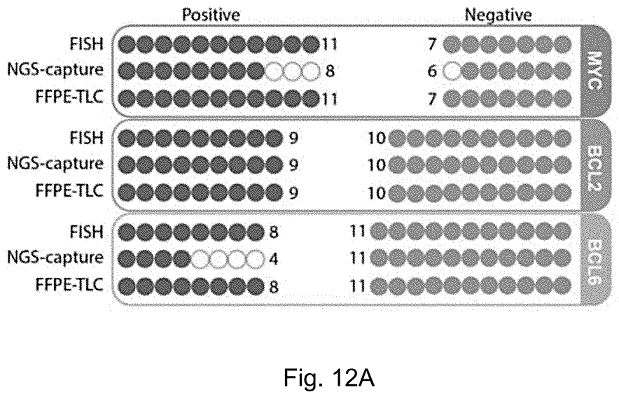

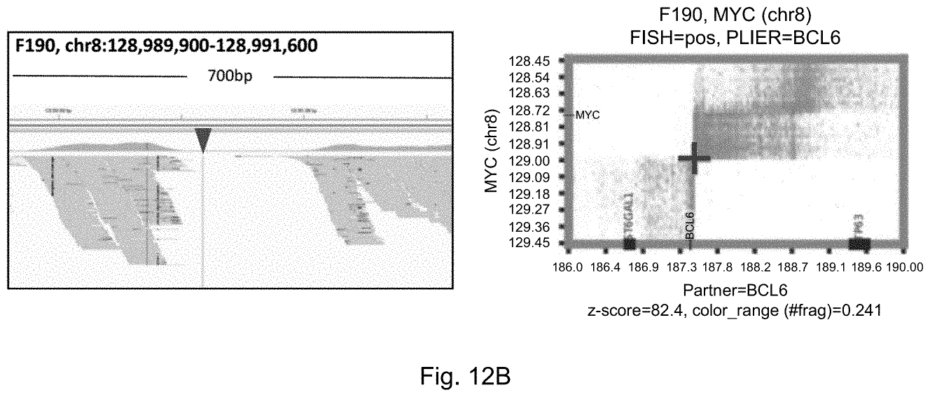

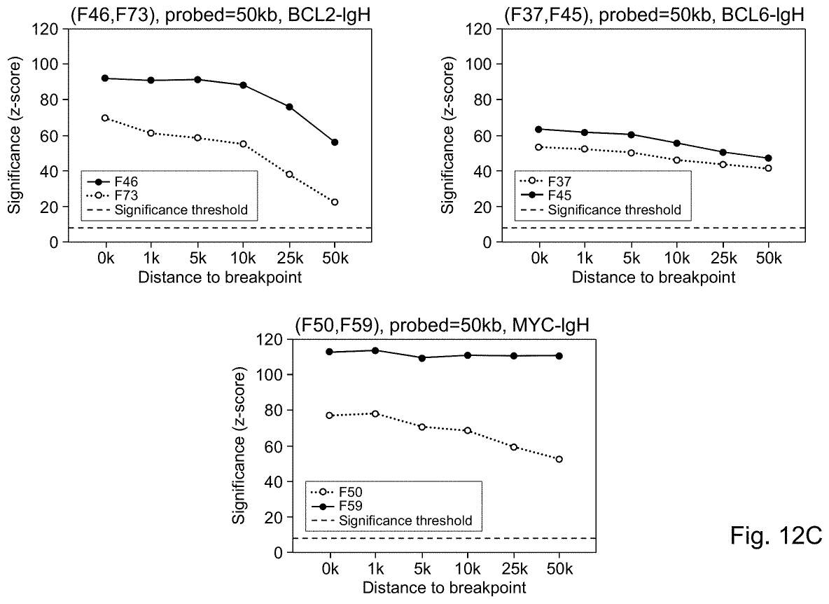

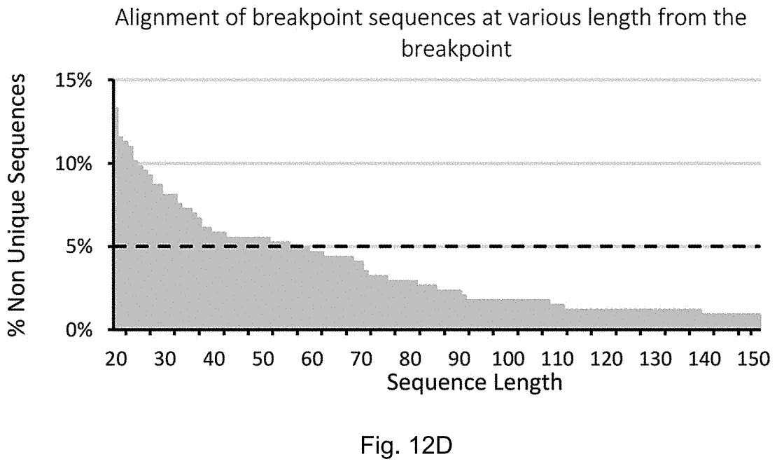

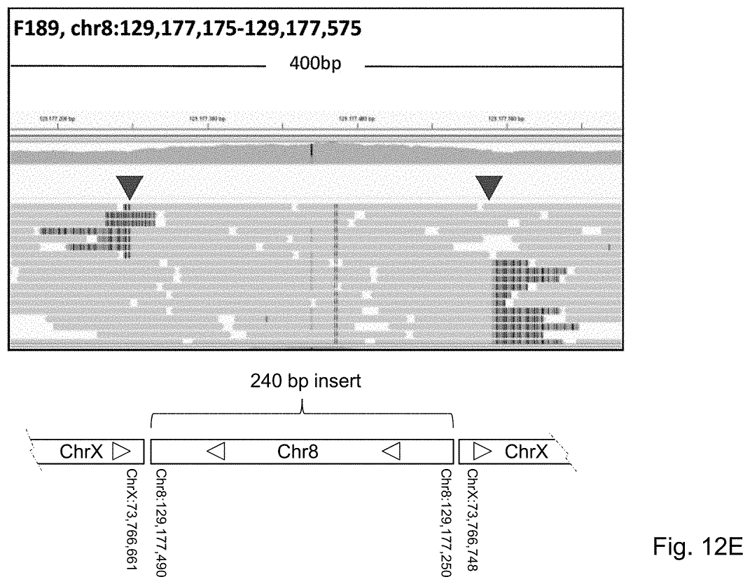

We also wished to compare FFPE-TLC to the targeted DNA capture-based sequencing methods (Capture-NGS) for the detection and analysis of structural variants in FFPE specimens⁵⁻⁷. For this, we compared Capture-NGS and FFPE-TLC performance on 19 FFPE samples that were part of a larger cohort of >200 FFPE samples previously analyzed by Capture-NGS. The selected samples included a subset in which the Capture-NGS results were discordant with the original FISH diagnoses. FIG. 12A shows the outcome of this comparison. Six out of six FFPE lymphoma samples in which Capture-NGS had failed to identify a total of seven FISH-reported translocations were confirmed by FFPE-TLC to carry the seven reported translocations (samples F190 (MYC and BCL6), F197 and F198 (MYC), F193 (BCL2), F188, F191, F192 (all BCL6)). In an effort to uncover the underlying reasons for which Capture-NGS had missed these rearrangements, we found that in three cases the actual breakpoint was outside the Capture-NGS probe targeted regions (F188, F197, F192). In one case (F190) FFPE-TLC demonstrated that the MYC and BCL6 rearrangements identified by FISH were actually a single MYC-BCL6 translocation. Capture-NGS failed to find a breakpoint fusion-read and therefore missed this rearrangement because the BCL6 breakpoint located outside the probe targeted region while the MYC breakpoint located in a repetitive sequence that could not be covered by probes (FIG. 12B). Thus, in cases where breakpoints occurred outside the probe-covered region, Capture-NGS failed to identify the rearrangement, whereas FFPE-TLC, as discussed, has no problem detecting such rearrangements. To illustrate this further, we reanalyzed datasets of six samples carrying a FISH-confirmed rearrangement with either BCL2 (2×), BCL6 (2×) or MYC (2×), but filtered the reads to exclusively consider captures made by a 50 kb interval placed at increasing distance from the mapped breakpoint: in all instances PLIER found the rearrangement with a very high confidence (FIG. 12C). In three other cases (F191, F192, F198) capture-NGS was not able to identify the rearrangement partner as it broke and fused at a non-unique sequence. To further assess the difficulty that NGS strategies may have in identifying rearrangements based on breakpoint fusion-read mapping, we analyzed the mappability of all breakpoint-flanking sequences found in this study, across different read lengths. FIG. 12D shows that around 5% of identified rearrangements would not be uniquely mappable and therefore missed even when reading 50 nucleotides into the partner sequence. Oppositely, there was one case for which capture NGS identified fusion-reads suggesting a MYC translocation, which was unconfirmed by FISH and by MYC immunohistochemistry, and where FFPE-TLC also not scored the translocation (F189). Detailed further analysis by PCR and sequencing revealed that this was a small insertion placing 240 base pair of chromosome 8 into chromosome X, but not affecting the MYC locus (FIG. 12E).

In conclusion, FFPE-TLC outperforms regular capture-NGS methods in the detection of chromosomal rearrangements. Capture-NGS relies on breakpoint fusion-read identification for the detection of rearrangements, which is severely hampered when breaks occur outside the probe-covered region and/or in repetitive DNA. FFPE-TLC, as we show, accurately finds these rearrangements because it analyzes the proximity-ligation pairs between a target gene and its rearrangement partner.

Discussion

We present here FFPE-TLC, a proximity-ligation based method for targeted identification of chromosomal rearrangements in clinically relevant genes in FFPE tumor samples. As an assay to be applied in the diagnostic setting, FFPE-TLC offers important advantages over FISH, the current gold standard for targeted rearrangement detection in lymphoma FFPE samples. Firstly, unlike FFPE-TLC, FISH is highly dependent on good quality tissue and cell morphology, which may be negatively impacted by necrosis, apoptosis and crush artifacts in resection specimens and by very limited material from core needle biopsy samples. We included core needle biopsy samples in this study, which showed that even very small samples yielded good quality FFPE-TLC results. Secondly, FISH results may give inconclusive results or lead to subjective interpretation in cases where aberrant numbers of FISH signals are seen per cell; FFPE-TLC offers the great benefit of objectively scoring rearrangements involving the selected target gene loci, based on a data analysis algorithm, PLIER. Thirdly, FFPE-TLC results provide much more detailed information on the rearrangement: not only does the method score whether or not the clinically relevant genes are intact or rearranged, as does FISH, it additionally identifies the rearrangement partner, the position of the breaks in relation to the genes involved, and, often, the fusion-read that describes the rearrangement at base pair resolution. Collecting this detailed information in relation to disease progression and treatment response is anticipated to improve diagnosis, prognosis and treatment of cancer patients. Translocation information at base pair level also provides an individualized tumor marker to enable the design of tumor-specific personalized assays for minimal residual disease testing. Finally, FFPE-TLC is more sensitive: to avoid false positive calling, FISH assessment generally uses a 10-20% cut point of aberrant signals as set by a normal control reference and caused by “cutting off” signals from 10-20 μm diameter tumor cells in 3-5 μm sections. FFPE-TLC reliably detects rearrangements even if present in only 5% of the cells, which makes it also an interesting method to apply to fusion gene detection in solid tumors.

Regular NGS-capture methods are also used to identify SVs, find fusion partners and provide detailed information on the rearrangement breakpoint, but also compared to these methods FFPE-TLC offers important advantages, particularly because it is not strictly reliant on successful pulldown and recognition of fusion reads. Rather, FFPE-TLC measures accumulated proximity-ligation events between chromosomal intervals flanking the breakpoint to identify a rearrangement. This, as we show, enables robust detection of rearrangements missed by regular NGS-capture methods, for example in cases when probes are not positioned close enough to the breakpoint for pulling down the fusion read, or when non-unique sequences flanking the breakpoint compromise fusion-read recognition.

A critical aspect of our study was the development of PLIER, our computational/statistical pipeline to objectively interrogate a FFPE-TLC dataset for rearrangement partners. Currently utilized fusion-read finders that process data produced from targeted NGS approaches often require a certain level of manual data curation, precluding fully automated and parallel data processing. In FFPE-TLC, PLIER enables automated identification of chromosomal rearrangements, from processing of sequenced FFPE-TLC libraries to the delivery of simple tables that include identified rearrangements. PLIER searches within each test sample for chromosomal intervals with significantly enriched densities of independently ligated fragments, without the need for comparison to a reference (or control) dataset. It thereby accounts for differences in the intrinsic signal to noise levels across samples, which is essential given the relatively large range of DNA quality from FFPE samples from different tissues, different hospitals and different archival storage times and conditions. Initially trained on a curated dataset of 6 samples and then applied to the full dataset of all samples, PLIER demonstrates to be very robust against varied levels of noise, and at the same time sensitive in detecting rearrangements across all 149 samples in our study.

The large number of rearrangements in malignant lymphomas that were uncovered in this study warrant consideration in light of the World Health Organization (WHO) classification of lymphomas. Currently, aggressive B-cell lymphomas with a combined MYC- and BCL2 and/or BCL6 translocations (so-called double-hit or triple-hit, DH/TH lymphomas) are classified as a separate entity, irrespective of morphological features. The rationale for this is not only found in the aim for “biologically meaningful classification”, but also in the characteristic poor clinical outcome that justifies a more intensified first-line treatment. More recently, in a very large series of such lymphomas, the Lunenburg Lymphoma Biomarker Consortium could show that this poor outcome is actually restricted to DH/TH lymphomas with an IG-partner to the MYC rearrangement, while all other contexts (MYC-single hit, non-IG partners) have a similar outcome to DLBCL without a MYC rearrangement. As a consequence, in the near future pathologists will be required to provide translocation status in aggressive B-cell lymphomas at this level of detail to support treatment decisions. Using FISH, 4 separate assays (BCL2,-BA (break-apart), BCL6-BA, MYC-BA, MYC-IGH-F (fusion)) are needed to diagnose DH/TH lymphomas, while still missing those cases that carry a MYC-IGL translocation since no commercial probes are available for MYC-IGL fusion FISH. Using FFPE-TLC, also this translocation context is diagnosed reliably in a single assay, which obviously improves time- and cost effectiveness. We identified 4 cases with MYC-IGL and one with MYC-IGK, of which one DH case (F264) in which clinical consequences would be immediate. We noted three cases of MYC-BCL6 fusion (F072, F190, F194) and two cases fusing MYC, BCL2 and IGH (F197, F274) that by FISH would not be identified as such and interpreted as a DH context in four cases and TH context in one. It is unknown, however, if a single translocation event activates both translocation partner genes and results in similar biological impact as two separate events. Similarly, both MYC and BCL6 are frequently translocated to genes with a likely biological impact on malignant B-cell behavior (e.g. TBL1XR1, CIITA, IKZF1, MEF2C, TCL1). Nevertheless, until now the impact of such fusion partners could not be studied in clinical settings.

In conclusion, FFPE-TLC combined with PLIER for objective rearrangement calling offers clear advantages over regular NGS-capture approaches and over FISH for the molecular diagnosis of lymphoma FFPE specimens. Future prospective studies should demonstrate how FFPE-TLC performs for other cancer types, like soft tissue sarcoma, prostate cancer and non-small cell lung carcinoma (NSCLC), which also frequently carry clinically relevant chromosomal rearrangements.

### REFERENCES

1. Muñoz-Mármol, A. M. et al. MYC status determination in aggressive B-cell lymphoma: the impact of FISH probe selection. Histopathology 63, 418-424 (2013).
2. Scott, D. W. et al. High-grade B-cell lymphoma with MYC and BCL2 and/or BCL6 rearrangements with diffuse large B-cell lymphoma morphology. Blood 131, 2060-2064 (2018).
3. Copie-Bergman, C. et al. MYC-IG rearrangements are negative predictors of survival in DLBCL patients treated with immunochemotherapy: a GELA/LYSA study. Blood 126, 2466-2474 (2015).
4. Cassidy, D. P. et al. Comparison Between Integrated Genomic DNA/RNA Profiling and Fluorescence In Situ Hybridization in the Detection of MYC, BCL-2, and BCL-6 Gene Rearrangements in Large B-Cell Lymphomas. American journal of clinical pathology 153, 353-359 (2020).
5. Chong, L. C. et al. High-resolution architecture and partner genes of MYC rearrangements in lymphoma with DLBCL morphology. Blood advances 2, 2755-2765 (2018).
6. McConnell, L. et al. A novel next generation sequencing approach to improve sarcoma diagnosis. Modern Pathology 33, 1350-1359 (2020).
7. Mendeville, M. et al. Aggressive genomic features in clinically indolent primary HHV8-negative effusion-based lymphoma. Blood 133, 377-380 (2019).
8. Lawson, A. R. et al. RAF gene fusion breakpoints in pediatric brain tumors are characterized by significant enrichment of sequence microhomology. Genome research 21, 505-514 (2011).
9. Hasty, P. & Montagna, C. Chromosomal Rearrangements in Cancer: Detection and potential causal mechanisms. Mol Cell Oncol 1 (2014).
10. Solomon, J. P., Benayed, R., Hechtman, J. F. & Ladanyi, M. Identifying patients with NTRK fusion cancer. Annals of oncology: official journal of the European Society for Medical Oncology 30, viii16-viii22 (2019).
11. Tachon, G. et al. Targeted RNA-sequencing assays: a step forward compared to FISH and IHC techniques? Cancer medicine 8, 7556-7566 (2019).
12. Zhu, G. et al. Diagnosis of known sarcoma fusions and novel fusion partners by targeted RNA sequencing with identification of a recurrent ACTB-FOSB fusion in pseudomyogenic hemangioendothelioma. Modern pathology: an official journal of the United States and Canadian Academy of Pathology, Inc 32, 609-620 (2019).
13. Pruis, M. A. et al. Highly accurate DNA-based detection and treatment results of <em>MET</em> exon 14 skipping mutations in lung cancer. Lung Cancer 140, 46-54 (2020).
14. Dekker, J., Rippe, K., Dekker, M. & Kleckner, N. Capturing chromosome conformation. Science (New York, N. Y.) 295, 1306-1311 (2002).
15. Chakraborty, A. & Ay, F. Identification of copy number variations and translocations in cancer cells from Hi-C data. Bioinformatics (Oxford, England) 34, 338-345 (2018).
16. de Vree, P. J. et al. Targeted sequencing by proximity ligation for comprehensive variant detection and local haplotyping. Nature biotechnology 32, 1019-1025 (2014).
17. Diaz, N. et al. Chromatin conformation analysis of primary patient tissue using a low input Hi-C method. Nature communications 9, 4938 (2018).
18. Dixon, J. R. et al. Integrative detection and analysis of structural variation in cancer genomes. Nature genetics 50, 1388-1398 (2018).
19. Harewood, L. et al. Hi-C as a tool for precise detection and characterisation of chromosomal rearrangements and copy number variation in human tumours. Genome biology 18, 125 (2017).
20. Simonis, M. et al. High-resolution identification of balanced and complex chromosomal rearrangements by 4C technology. Nature methods 6, 837-842 (2009).
21. Troll, C. J. et al. Structural Variation Detection by Proximity Ligation from Formalin-Fixed, Paraffin-Embedded Tumor Tissue. The Journal of molecular diagnostics: JMD 21, 375-383 (2019).
22. Akasaka, T., Lossos, I. S. & Levy, R. BCL6 gene translocation in follicular lymphoma: a harbinger of eventual transformation to diffuse aggressive lymphoma. Blood 102, 1443-1448 (2003).
23. Wang, S. et al. HiNT: a computational method for detecting copy number variations and translocations from Hi-C data. Genome biology 21, 73 (2020).
24. Krzywinski, M. I. et al. Circos: An information aesthetic for comparative genomics. Genome research (2009).
25. Joos, S. et al. Variable breakpoints in Burkitt lymphoma cells with chromosomal t(8; 14) translocation separate c-myc and the IgH locus up to several hundred kb. Human Molecular Genetics 1, 625-632 (1992).
26. Ohno, H. et al. Diffuse large B-cell lymphoma carrying t(9; 14)(p13; q32)/PAX5-immunoglobulin heavy chain gene is characterized by nuclear positivity of MUM1 and PAX5 by immunohistochemistry. Hematological Oncology 38, 171-180 (2020).
27. Gascoyne, D. M. & Banham, A. H. The significance of FOXP1 in diffuse large B-cell lymphoma. Leukemia & Lymphoma 58, 1037-1051 (2017).
28. Shi, J. et al. High Expression of <em>CPT1A</em> Predicts Adverse Outcomes: A Potential
Therapeutic Target for Acute Myeloid Leukemia. EBioMedicine 14, 55-64 (2016).
29. Ichikawa, S. et al. Association between BACH2 expression and clinical prognosis in diffuse large B-cell lymphoma. Cancer Science 105, 437-444 (2014).
30. Salaverria, I. et al. The CBFA2T3/ACSF3 locus is recurrently involved in IGH chromosomal translocation t(14; 16)(q32; q24) in pediatric B-cell lymphoma with germinal center phenotype. Genes, Chromosomes and Cancer 51, 338-343 (2012).
31. van de Werken, H. J. G. et al. Robust 4C-seq data analysis to screen for regulatory DNA interactions. Nature methods 9, 969-972 (2012).
32. Krijger, P. H. L., Geeven, G., Bianchi, V., Hilvering, C. R. E. & de Laat, W. 4C-seq from beginning to end: A detailed protocol for sample preparation and data analysis. Methods 170, 17-32 (2020).
33. Li, H. Aligning sequence reads, clone sequences and assembly contigs with BWA-MEM. arXiv preprint arXiv: 1303.3997 (2013).
34. Geeven, G., Teunissen, H., de Laat, W. & de Wit, E. peakC: a flexible, non-parametric peak calling package for 4C and Capture-C data. Nucleic Acids Research 46, e91-e91 (2018).
35. Collette, A. Python and HDFS: unlocking scientific data. (“O'Reilly Media, Inc.”, 2013).
36. de Ridder, J., Uren, A., Kool, J., Reinders, M. & Wessels, L. Detecting Statistically Significant Common Insertion Sites in Retroviral Insertional Mutagenesis Screens. PLOS Computational Biology 2, e166 (2006).

Materials and Methods

Patient samples: This retrospective study used a set of 129 archival B-cell Non-Hodgkin lymphoma tissue samples, which were selected by the respective sites, and may therefore not represent an entirely random selection of samples in the respective sites. The corresponding lymphoma patients had been diagnosed between 2007 and 2019 at the University Medical Centre Utrecht, Amsterdam University Medical Centre—location VUMC, Laboratorium Pathologie Oost-Nederland, Leiden University Medical Centre and University Medical Centre Groningen and their affiliated hospitals. They had been mostly diagnosed as DLBCL, but also Burkitt, follicular and marginal zone lymphomas and some other diagnoses were included. 20 Non-lymphoma control samples were also analyzed, mostly reactive lymph node samples and tonsillectomy specimens. Formalin-fixed and paraffin-embedded (FFPE) tissue samples were obtained using standard diagnostic procedures. Per patient, 1 or more 10 μm scrolls or 4 μm unstained sections of the FFPE tissue blocks were provided for FFPE-TLC analysis in tubes or on slides. The study was performed in accordance with the local institutional board requirements and all relevant ethical and privacy regulations were followed during this study.

Molecular analysis: All patient samples had been analyzed with routine FISH with break-apart probes and fusion-probes in selected cases, in the majority of cases for all 3 genes BCL2 (Cytocell LPS028; Vysis Abbott 05N51-020; IGH/BCL2 Dual Fusion Vysis Abbott 05J71-001), BCL6 (Cytocell LPH 035; Vysis Abbott 01N23-020) and MYC (Cytocell LPS 027; Vysis Abbott 05J91-001; IGH/MYC/CEP 8 Dual Fusion Vysis Abbott 04N10-020). A subset of 19 samples had also been analyzed with a Capture-NGS method as developed by the Amsterdam University Medical Centre—location VUMC team. A detailed description of this approach is provided in the Supplementary

### Materials & Methods.

FFPE-TLC library preparation: In brief, single FFPE sections were supplied by the medical centers in this study as scrolls in 1.5 ml vials or on slides. If a slide was provided, the contained material in the slide was scraped and transferred to a 1.5 ml vial. Excessive paraffin was removed by a 3-minute 80° C. heat treatment, followed by a centrifugation step after which the tissue was disrupted and homogenized by sonication using a M220 Focused-ultrasonicator (Covaris). Samples were primed for enzymatic digestion through incubation with 0.3% SDS for 2 hours at 80° C., then digested with NlaIII (a 4 base pair cutter restriction enzyme; NEB) at 37° C. for 1 hour, and finally ligated at room temperature for 2 hours with T4 DNA ligase (Roche). Next, a complete reverse crosslinking was done by an overnight incubation at 80° C. and the DNA was purified using isopropanol precipitation and magnetic bead separation. Following elution, 100 ng of the prepared material was fragmented to 200-300 bp (M220 Focused-ultrasonicator, Covaris) and subjected to NGS library prep (Roche Kapa Hyperprep, Kapa Unique Dual indexed adapter kit). A total of 16-20 independently prepared libraries were equimolar pooled with a total mass of 2 μg and subjected to hybridization with the capture probe pool, wash steps and PCR amplification using the Roche Hypercap reagents and workflow according to the manufacturer's instructions. Paired-end sequencing was done on an Illumina Novaseq 6000 sequencing machine. All proximity-ligation libraries were sequenced deeper than deemed necessary. The samples with lowest coverage were sequenced to a read depth of around 20M, which invariably was sufficient for rearrangement detection.

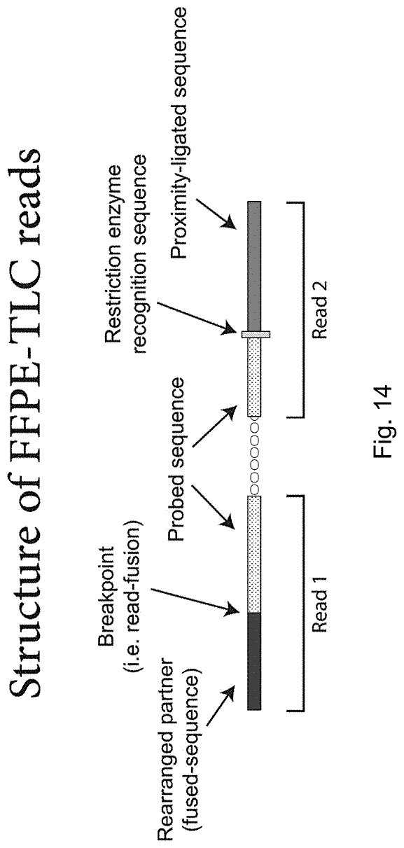

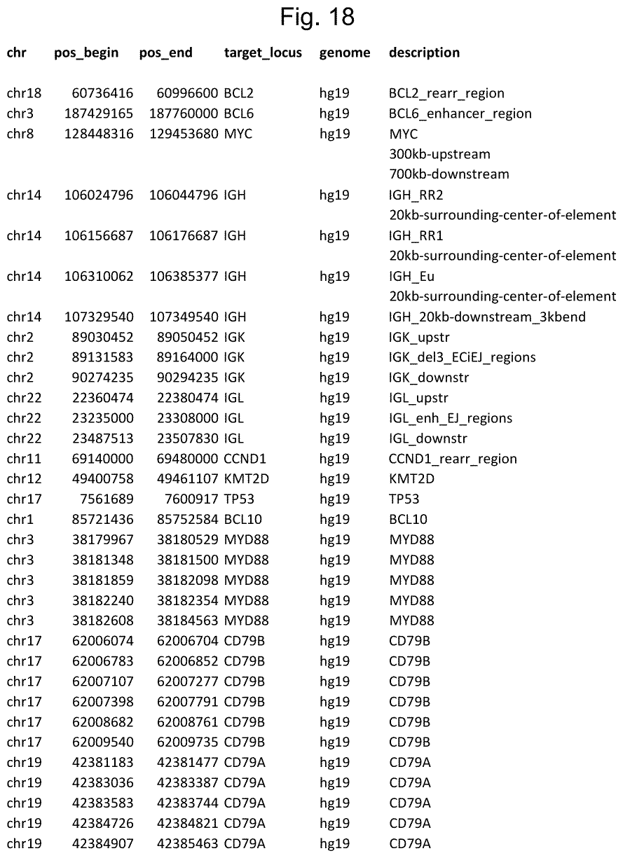

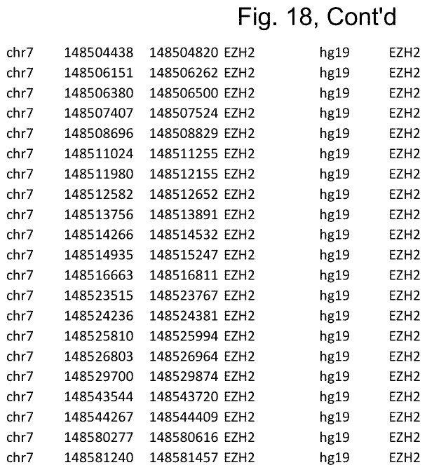

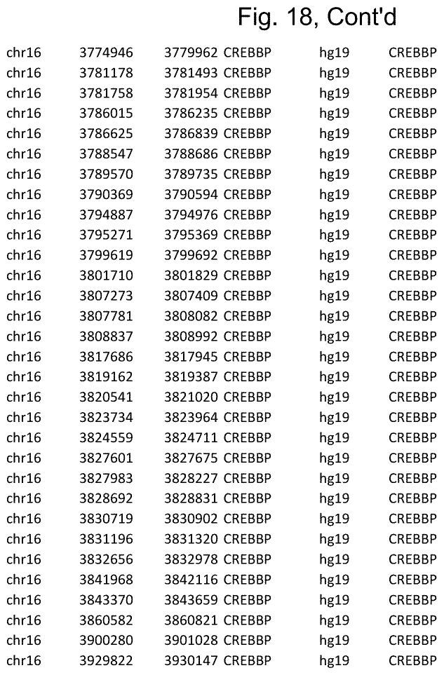

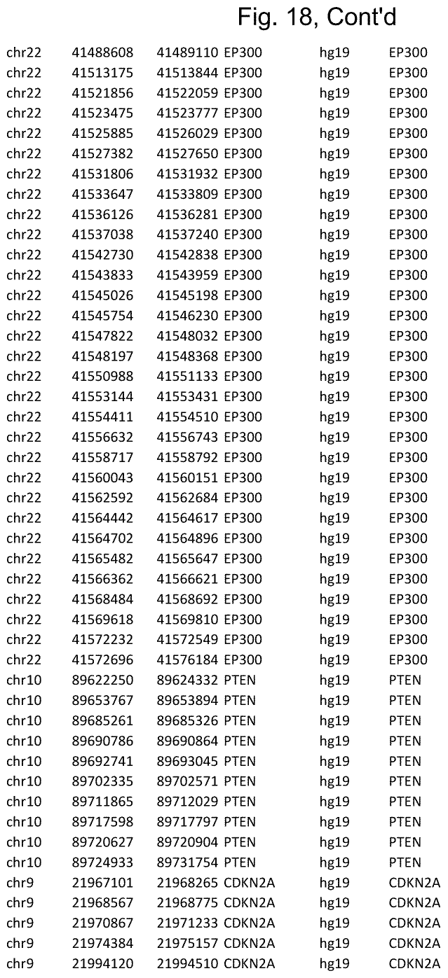

FFPE-TLC data processing: Sequenced reads from individual samples (i.e. patients) were mapped to the human genome (hg19) using BWA-MEM (settings: -SP -k12 -A2 -B3) in paired-end mode³³. BWA-MEM aligner allowed “split-mapping” in which a single read can be mapped into multiple fragments (i.e. separate regions) in the genome. This was essential to map FFPE-TLC data, as each sequenced read in FFPE-TLC may contain multiple fragments mapping to varied locations in the genome (see FIG. 14). Any fragments with mapping quality (MQ) above zero were considered as mapped, as is commonly done for proximity-ligation data processing^{32, 34}. Reads were assigned to their related target gene or “viewpoint” (i.e. a probe set such as MYC, BCL2, etc.) based on their fragment's overlap with the viewpoint's coordinates (FIG. 18 for probe set coordinates). A read was discarded if it did not overlap with any viewpoint. In cases with fragments within a read that had overlap with multiple viewpoints, the read was assigned to the viewpoint with the largest overlap. As a result of this procedure, for each combination of sample and viewpoint, an independent FFPE-TLC alignment file (BAM) was produced.

The reference genome was split in silico into “segments” based on the recognition sequence of NlaIII restriction enzyme (CATG) where each segment starts and ends with an NlaIII recognition site. Mapped fragments were then overlaid on the segments. Due to rare alignment errors, more than one fragment within a read can overlap a segment. In such a case, only one fragment was counted for that particular segment and extra overlapping fragments on that read were ignored. We used HDFS format³⁵ to store FFPE-TLC datasets which is a cross-platform and cross-language file storage standard and therefore delivers convenience to future users of FFPE-TLC.

Rearrangement identification: See de Ridder et al.³⁶, which aims to identify more than expected enrichment of a signal (i.e. coverage) across the genome. In a given FFPE-TLC dataset, PLIER initially splits the reference genome into equally spaced genomic intervals (e.g. 5 kb or 75 kb bins) and then calculates for every interval a “proximity frequency” that is defined by the number of segments within that genomic interval that are covered by at least one fragment (i.e. a proximity-ligation product), see FIG. 6 for a schematic overview on the entire procedure. “Proximity scores” are then calculated by Gaussian smoothing of proximity frequencies across each chromosome to remove very local and abrupt increase (or decrease) in proximity frequencies that are most likely spurious. Next, an expected (or average) proximity score and a corresponding standard deviation are estimated for genomic intervals with similar properties (e.g. genomic intervals present on trans chromosomes) by in silico shuffling of observed proximity frequencies across the genome followed by a Gaussian smoothing across each chromosome. Finally, a z-score is calculated for every genomic interval using its observed proximity score and the related expected and standard deviation of proximity scores. Finally, by combining z-scores calculated from multiple scales (i.e. interval widths such as 5 kb and 75 kb), a scale-invariant enrichment score is calculated (see Enrichment score estimation and Parameter optimization for PLIER sections for details). This scale-invariant enrichment score is used to recognize genomic intervals with elevated clustering of observed ligation products.

For genomic intervals present on cis chromosomes, we first corrected the known elevated proximity frequencies of genomic intervals adjacent to the targeted loci. To this end, for a given FFPE-TLC dataset we initially excluded the probed area as well as the surrounding +/−250 kb area. Then, we performed a Gaussian smoothing (σ=0.75, span=31 intervals) on proximity frequencies on both sides of the probed area until the chromosome ends. Next, inspired by peakC³⁴, we performed an Isotonic-regression on the smoothed proximity frequencies. For each cis-interval we considered the difference between its smoothed proximity frequency and the corresponding Isotonic-regression prediction value as its proximity score. This procedure ensures that the known elevation of proximity scores in genomic intervals adjacent to the targeted (or probed) loci is accounted for. Finally, enrichment scores for cis intervals were calculated following a shuffling procedure similar to trans intervals (described above). We discarded cis-rearrangements identified in the +/−3 mb region around the viewpoint (i.e. closer than 3 mb to the viewpoint measured across the linear chromosome) to make sure the true 3D interactions between the viewpoint and its vicinity is not considered as rearrangement.

It is worth noting that the above statistical approach works well when a FFPE-TLC dataset is not sparse and is at least minimally populated with independent ligation products (i.e. coverage on diverse genomic segments in the genome). However, a sparse FFPE-TLC can arise from a library prepared with poor sample (tissue) quality, DNA extraction, low digestion or ligation efficiency or other difficulties in library preparation. In such cases, only a minimal number of genomic intervals in the genome will have a proximity score above zero. As a result, the utilized permutation strategy (i.e. random shuffling of intervals) will underestimate the true expected proximity score and therefore many intervals with proximity score above zero will be falsely considered as enriched. To remedy this issue, we considered a complementary permutation approach in which we only swapped the genomic intervals with proximity frequency above zero (instead of random shuffling of all intervals) and then calculated the corresponding z-scores by comparing the observed and expected proximity scores that are calculated using the swapping permutation strategy. For each genomic interval we took the minimum z-score between the shuffling and swapping permutations as the final z-score for that particular genomic interval. This addition limited the number of false-positive calls even in a sparse FFPE-TLC dataset and makes PLIER suitable for FFPE-4C experiments as well. In all permutations, we repeated the shuffling or swapping 1000 times to estimate the corresponding expected and standard deviation of proximity scores.

It is important to note that in this approach, we do not correct for known biases such as GC content, mappability, segment or restriction site density (i.e. number of restriction sites per interval) or a number of other known factors that could influence captured proximity frequencies. Owing to PLIERs flexibility, these parameters can be considered in the background estimation by only swapping (or shuffling) intervals that have similar chromatin compartment, GC content, restriction site density, etc. Nonetheless, our preliminary analyses did not show a considerable improvement when these parameters were corrected for in the background estimation and therefore, we opted for simplicity of the model which in turn reduces the computational demand of PLIER. This decision was especially important because we aimed to produce a light-weight pipeline that is suitable to be implemented in a clinical setting with minimal computational requirements. PLIER's source code will be available for download from Github at https://github.com/deLaatLab/PLIER.

Enrichment score estimation: For a given sample (e.g. a patient) and viewpoint (e.g. BCL2) and genomic interval width (e.g. 5 kb), we initially selected genomic intervals that showed z-score above 5.0 and merged the neighbor selected intervals if they were closer than 1 mb. We took the 90-percentile z-score values of the merged intervals as their integrated z-score. To estimate the “scale-invariant” enrichment score from multiple interval widths (e.g. 5 kb and 75 kb), we grouped merged intervals that were closer than 10 mb and took the z-score value of the intervals with largest scale (75 kb in this case) as the final enrichment score. Each collection of merged intervals across scales is referred to as a “call” in this study.

Parameter optimization for PLIER (i.e. training phase): To identify PLIER's optimal parameters, we used a collection of six FFPE-TLC samples, three lymphoma (“positive”) and three control (“negative”) samples. Specifically, three lymphoma samples (i.e. F73, F37 and F50) were included which, based on FISH (the gold standard), were expected to have a single rearrangement in BCL2, BCL6 or MYC, respectively while lacking rearrangement in the other two genes. The other three “negative” datasets (i.e. F29, F30 and F33) were control datasets for which no rearrangements were expected in any of the three genes. We limited the optimization to BCL2, BCL6 and MYC genes as we only had clinical/diagnosis FISH data for these genes. We also included dilution (i.e. 5%, 1% and 0.2%) experiments of the three lymphoma samples (i.e. F73, F37 and F50) in the optimization procedure. Taken together, we had 12 positive cases (the 3 original patients, plus 3 additional dilution samples for each patient) for which PLIER should identify a rearrangement (i.e. “true positives” set) and 33 negative cases (3 control samples each with three genes, plus the two non-rearranged genes in 12 lymphoma samples) for which PLIER should not identify any rearrangement across the genome (i.e. “true negative” set). Apart from the correctly identified rearrangements, any extra rearrangement found in the positive cases across the genome were also considered as “false-positive” rearrangements. As performance measure we used Area Under Precision Recall (AUC-PR) instead of Area Under the Curve as we potentially had more negative cases than positive cases (i.e. unbalanced class frequencies).

For an effective performance of PLIER's statistical framework, several parameters need to be optimally defined. We performed a massive parameter sweep using High Performance Computing (HPC) of University Medical Center Utrecht to identify the optimal parameters for PLIER. These parameters include: Gaussian smoothing degree (σ=0.1, 0.25, 0.5, 0.75, 1.0, 1.5, 2.0, 2.5, 3.0, 3.5, 4.0), number of genomic intervals that Gaussian kernel spans (#step=11, 21, 31, 41, 51, 61) and genomic interval widths (width=5 kb, 10 kb, 25 kb, 50 kb, 62 kb, 75 kb, 100 kb). For interval widths, we also tested if combining multiple interval widths (i.e. scale-invariant enrichment scores) would perform better. Additionally, to identify how the z-score of merged intervals (i.e. the intervals within 1 mb neighborhood of each other) should be integrated, we considered experimenting with maximum, 90 percentile and median operators.

After the parameter sweep, we identified the followings as optimal parameters of PLIER: Gaussian smoothing σ=0.75, Gaussian kernel span #step=31, interval widths=5 kb+75 kb (i.e. both z-score should be above 5.0) and 90 percentile of z-scores of neighbor (<1 mb) intervals being merged as their final z-score. Finally, a significance threshold needed to be estimated to consider a call to be significantly enriched. By setting the maximum False Discovery Rate (FDR) as 1%, we reached significance of 8.0 as the optimal significance threshold for enrichment scores of trans-intervals. Due to computational constraints and limited availability of diagnostic data, we only optimized PLIER parameters for trans-intervals of BCL2, BCL6 and MYC. We then used these parameters (without further optimization) for trans-intervals of other genes in the study (i.e. IGH, IGL and IGK). For cis-intervals of all genes in our study, we again used the aforementioned parameters, with the exception of the significance threshold. For these calls we took a conservative approach of much higher significance threshold (i.e. >16.0). Each output call from PLIER consisted of two genomic coordinates that indicate the boundary in which the scale-invariant enrichment score was above the significance threshold.

Amplification detection: Although FFPE-TLC is not designed to identify amplifications, repeated rearrangements identified by PLIER from different probe sets but in the same sample and region can be indications of amplification events in that region. To leverage this prospect, we focused on the three primary genes in our study (i.e. MYC, BCL2 and BCL6) for which relatively large areas were probed (see FIG. 18 for details). For each sample, we asked if a particular rearrangement (i.e. in the same region) is reported from more than one gene. An example of such amplification identified by PLIER is depicted in FIG. 9E. Of note, lymphoma samples could potentially harbor double hit rearrangements (e.g. BCL2 and MYC) specifically to the IGH area. To avoid calling such a rearrangement as amplification events, we excluded calls to the IGH area from amplification detection analysis.

Blacklisted areas: We noted that our IGL and IGK probe sets tend to repeatedly identify specific regions in the genome. We observed such calls even in our control samples for which no rearrangements were expected to be present. Specifically, our IGL probe set frequently identified chr9:131.5-132.5 mb and our IGK probe set frequently identified chr22:22-24 mb region of the human (hg19) genome. It is worth noting that the chr22:22-24mb area harbors the IGL gene and therefore such calls could potentially be interesting to investigate further. However, we noted that the corresponding IGL viewpoints did not identify IGK reciprocally. Consequently, we considered the elevation of enrichment scores to be due to a high sequence similarity between IGL and IGK that is likely to cause misalignments during the mapping procedure. Taken together, we considered both areas as off-target bindings of IGK and IGL probes, respectively and ignored any rearrangements identified by these two probe sets in these areas.

Fusion-read identification: To identify fusion-reads in a given FFPE-TLC dataset (e.g. MYC), we collected split-alignments (i.e. individual read sequences that mapped to multiple areas in the genome). Then, the split-alignments that referred to enzymatic digestion in FFPE-TLC were filtered out by discarding the split-alignments that fused at a restriction enzyme recognition site in the genome (+/−1 base pair). The split-alignments that occurred at the rearranged coordinates (identified by PLIER) were manually checked in IGV to confirm the existence of read-fusions.

Fusion-read mappability: The identified breakpoint coordinates from the fusion reads were used in the mappability analysis to extract the corresponding sequences from the reference genome. In total 347 sequences of 151 bp (equal to the sequencing read length) upstream and downstream of the breakpoints were extracted from the reference genome. These 347 sequences were aligned using blastn (settings: -perc_identity 80 -dust no -evalue 0.1) at different sequence lengths from 20 to 151, using a step size of 1 bp. The blast results were parsed to count the sequences with exact hits at each length; if exactly one hit, the sequence is considered unique, if multiple hits the sequence is considered non-unique. The fraction of non-unique sequences was plotted in a bar graph.

Confirmation of the 240 bp chr8 insertion into chrX in sample F189: A 2×20 cycles nested PCR was performed on control DNA and DNA isolated from sample F189 (Nebnext Q5 mix, NEB) using two primers for the initial PCR flanking the insertion on chrX (Fwd: ATTTTGATCGGCTTAGACCA, Rev: GGTTGATCAAAGCCAGTC) and 2 primers for the nested PCR (Fwd: GTCCAGCTTTGTCCTGTATT, Rev: GTCATGGCTGGTCAAGATAG. PCR products were separated on agarose gel, showing the expected sized product with insertion had been formed only for sample F189 (data not shown). For further confirmation the primary PCR products were amplified in the same nested PCR but now including Illumina sequencing adapters and an index sequence (Fwd: GTGACTGGAGTTCAGACGTGTGCTCTTCCGATCTGTCCAGCTTTGTCCTGTATT, Rev: ACACTCTTTCCCTACACGACGCTCTTCCGATCTGTCATGGCTGGTCAAGATAG) and subjected to sequencing (Illumina MiniSeq). Data availability: All sequencing data used in this study were mapped to the reference genome (hg19) and are available through the European Genome-phenome Archive.

Supplementary Materials & Methods: Capture-NGS DNA isolation, library preparation and sequencing: DNA was extracted from 3-10×10 μm FFPE sections using the QIAamp DNA FFPE Tissue Kit (Qiagen, Hilden, Germany) according to manufacturer's protocol. Peripheral blood DNA was extracted using the QIAamp Blood Mini Kit (Qiagen, Hilden, Germany) according to the manufacturer's spin protocol. Isolated DNA was quantified using a Qubit 2.0 Fluorometer using the QubitBR kit (Thermo Fisher Scientific, Carlsbad Calif., USA) and 250-800 ng in a total volume of 130 μl was fragmented with a Covaris S2 or ME220 (Covaris Inc, Woburn Mass., USA) for 6 minutes at 200 cycles per burst to an average size of 180-220 bp for the Covaris S2 and for 3 minutes at 1000 cycles per burst to an average size of 250-300 bp. DNA concentrations and the fragmentation profile/size distribution were determined with a 2100 bioanalyzer using the Agilent DNA 1000 kit (Agilent Technologies, Santa Clara, Calif.). 250 ng of 180-220 or 250-300 bp fragmented DNA was used to create NGS libraries with the KAPA library preparation kit (KAPA Biosystems, Wilmington Mass., USA). In short, the DNA ends were repaired (20° C. for 30 minutes) and single A-tails were ligated (30° C. for 30 minutes). Subsequently, uniquely indexed adapters (Roche Nimblegen, Madison Wis., USA; IDT, Coralville Iowa, USA) were ligated overnight (16° C.) after which size selection was performed to retain fragments between 250-450 bp. DNA was amplified for seven polymerase chain reaction (PCR) cycles. An aliquot of the created DNA libraries was subjected to targeted capture. A capture panel was designed with NimbleGen design software (Roche). The capture panel covers exons of ˜350 genes (˜1.5 Mb) for mutation analysis and multiple chromosomal regions (including genes, introns and intergenic regions; ˜1.5 Mb) for translocation analysis (Roche order ID 0200204534, ID 43712, and ID 1000002633). Capture was performed according to NimbleGen EZ SeqCap library protocol V5.1 (Roche Nimblegen, Madison Wis., USA). Per capture, DNA of eight libraries were equimolarly pooled together in one tube to a total of 1 μg DNA. Probe hybridization was performed overnight at 47° C. Pools were amplified for 14 PCR cycles. Three pools were equimolar pooled and loaded together on one sequence lane and sequenced 125 bp or 150 bp paired-end on a HiSeq 2500 or 4000 respectively.

Alignment of sequence reads: NGS reads were de-multiplexed with Bcl2fastq (Illumina). Adapters and poor quality bases were trimmed with SeqPurge (-min len 20; v0.1-104). Reads were aligned against the human reference genome (hg19) with BWA mem (-M -R; v0.7.12) (Heng 2013). Read realignment with ABRA (v0.96)(Mose et al. 2014) was used to improve alignment accuracy. The aligned bamfiles were sorted on query name with Sambamba (v0.5.6), and duplicate reads were flagged with Picardtools MarkDuplicates (v2.4.1), using the setting ASSUME SORT ORDER=queryname. This setting is required to mark duplicate secondary alignments in addition to duplicate primary alignments. (Tarasov et al. 2015; ‘Picard tools’). Next, reads were sorted by coordinate (Sambamba) for compatibility with the rest of the data analysis pipeline.

Structural variant analysis: The part of the pipeline for structural variant analysis, including translocations, inversions, deletions, insertions and duplications, was created in the workflow management system Snakemake (Köster and Rahmann 2012). To obtain high sensitivity and specificity 4 translocation detection algorithms were combined: BreaKmer (v.0.0.4)(Abo et al. 2015), GRIDS S (v.1.4.2)(Cameron et al. 2017), NovoBreak (v.1.1.3)(Chong et al. 2017) and Wham (v.1.7.0)(Kronenberg et al. 2015). These were selected based upon the following criteria. 1. Possibility to detect translocations 2. Works with paired end Illumina sequencing data with short insert size. 3. Usable on targeted sequencing data 4. Documentation available 5 Maintained till at least 2017. BreaKmer, GRIDSS and novoBreak were executed with default settings. Wham was executed with mapping quality of 10 (-p) and base quality of 5 (-q). For compatibility with BreaKmer, chromosome-prefixes were removed from the bamfile. BreaKmer requires a target bed file containing the regions of interest for translocation detection, to reduce assembly time and to obtain higher accuracy, the translocation targets were divided in regions of 5 kb in the target bed file.

To be able to combine the output of these tools, the output was converted in R (v.3.4.1) to be comparable between tools, and gene annotation was added. To remove noise, filters were applied. In subsequent order the following SVs were removed from the data:

SVs with both breakpoints off-target, further than 300 bp outside the capture probe location.
Duplicate SVs with exactly the same breakpoints detected with the same tool.
SVs not meeting set thresholds for the tool. For BreaKmer, at least 4 split reads and 3 discordant reads, for Wham at least 8 reads (sum of discordant and split reads), for GRIDSS a quality score above 450 and for novoBreak an average coverage of at least 4 high mapping quality translocation reads. SV output of the four tools were combined and SVs detected by only one tool were removed. Hence, only SVs recognized by at least 2 tools were included. Therefore, breakpoints that lie within a 10 bp margin were considered to be the same SV.

Blacklist: Examination of the results showed multiple often recurrent SVs. Manual inspection of these events in the integrative genome viewer (IGV) taught us that those SVs were artifacts of different origins. Part of the artifactual SVs were a consequence of highly repetitive regions in the genome, others were introduced by partly homologous regions. Furthermore, some common germ-line SVs, especially small indels, were detected in the data. To remove those problematic regions from the output, a blacklist was created based on a panel of 25 non-tumor samples, (12 blood samples, 4 FFPE hyperplasia lymph node, 6 FFPE reactive lymph node and 3 FFPE epithelial tissues). For these 25 samples SV detection was performed following the exact same DNA, isolation, preparation and sequencing as well as the four selected detection tools with the same settings. Common breakpoint locations detected in at least 2 non-tumor samples within a margin of 10 bp were added to the blacklist using Bed-tools multi-inter (v0.2.17). Blacklisted areas less than 50 bp apart were merged to one region with Bedtools merge. SVs with one of the breakpoints within the blacklisted regions were removed from the SV detection output. Remaining SVs were manually inspected in IGV.

1. A method of detecting a chromosomal rearrangement involving a genomic region of interest, using a dataset of DNA reads, the dataset comprising DNA reads representing genomic fragments being in nuclear proximity to the genomic region of interest, the method comprising
assigning an observed proximity score to each of a plurality of genomic fragments of a genome, the observed proximity score of each genomic fragment being indicative of a presence in the dataset of at least one DNA read in nuclear proximity to the genomic region of interest and comprising a sequence corresponding to the genomic fragment;
assigning an expected proximity score to each of at least one genomic fragment of the plurality of genomic fragments, based on the observed proximity scores of the plurality of genomic fragments, wherein the expected proximity score comprises an expected value of the proximity score of the at least one of the plurality of genomic fragments; and
generating an indication of a likelihood that said at least one genomic fragment of the plurality of genomic fragments is involved in a chromosomal rearrangement, based on the observed proximity score of said at least one genomic fragment of the plurality of genomic fragments and the expected proximity score of said at least one genomic fragment of the plurality of genomic fragments.

2. The method of claim 1, wherein the assigning the expected proximity score to said at least one genomic fragment comprises:
determining a plurality of related proximity scores based on the observed proximity scores of a plurality of related genomic fragments, wherein the related genomic fragments are related to said at least one genomic fragment according to a set of selection criteria; and
determining the expected proximity score of said at least one genomic fragment based on the plurality of related proximity scores.

3. The method of claim 2, wherein the determining the plurality of related proximity scores comprises:
generating a plurality of permutations of the observed proximity scores, thereby identifying a corresponding plurality of permuted observed proximity scores of each of the genomic fragments, wherein generating a permutation comprises swapping the observed proximity scores of randomly chosen genomic fragments that are related to each other according to the set of selection criteria.

4. The method of claim 3, wherein
determining each related proximity score of said at least one genomic fragment further comprises aggregating the permuted observed proximity scores of a permutation by aggregating the permuted observed proximity scores of the genomic fragments in a genomic neighborhood of said at least one genomic fragment within the permutation to obtain an aggregated permuted observed proximity score of the genomic fragment for each permutation.

5. The method of claim 4,
further comprising aggregating the observed proximity scores of the genomic fragments in the genomic neighborhood of said at least one genomic fragment, to obtain an aggregated observed proximity score of said at least one genomic fragment,
wherein the generating the indication of whether said at least one genomic fragment of the plurality of genomic fragments is involved in a chromosomal rearrangement is performed based on the aggregated observed proximity score of the at least one genomic fragment and the expected proximity score of the at least one genomic fragment.

6. The method of claim 5,
further comprising aggregating the observed proximity scores of the genomic fragments in the genomic neighborhood of each genomic fragment, to obtain an aggregated observed proximity score of each genomic fragment,
wherein the permutations are generated based on the aggregated observed proximity score of each genomic fragment, and
wherein the generating the indication of whether said at least one genomic fragment of the plurality of genomic fragments is involved in a chromosomal rearrangement is performed based on the aggregated observed proximity score of the at least one genomic fragment and the expected proximity score of the at least one genomic fragment.

7. The method of claim 5, wherein the steps of aggregating the proximity scores, assigning the expected proximity score, and generating the indication of a likelihood that said at least one genomic fragment of the plurality of genomic fragments is involved in a chromosomal rearrangement are iterated for a plurality of different scales, wherein in each iteration a size of the genomic neighborhood is based on the scale.

8. The method of claim 1,
wherein determining the expected proximity score of said at least one genomic fragment comprises combining the plurality of related proximity scores of said at least one genomic fragment to determine for example an average and/or a standard deviation.

9. The method of claim 1, wherein the assigning the observed proximity score to each of the plurality of genomic fragments comprises:
assigning an observed proximity frequency to a plurality of genomic fragments of a genome, the observed proximity frequency being indicative of a presence in the dataset of at least one DNA read of the corresponding genomic fragment; and
computing each observed proximity score by combining the observed proximity frequencies in a genomic neighborhood of each genomic fragment, for example by binning the observed proximity frequencies, preferably wherein the observed proximity frequency comprises a binary value indicating whether the DNA read corresponding to the genomic fragment is present in the dataset or a value indicative of a number of DNA reads corresponding to the genomic fragment in the dataset.

10. The method of claim 1, wherein the providing the dataset of DNA reads comprises
a. determining the genomic region of interest in the reference genome;
b. performing a proximity ligation assay to generate a plurality of proximity ligated fragments;
c. sequencing the proximity ligated fragments;
d. mapping the sequenced proximity ligated fragments to a reference genome;
e. selecting a plurality of the sequenced proximity ligated fragments that include a sequence that is mapped to the genomic region of interest; and
f. detecting genomic fragments that are ligated to the genomic region of interest in at least one of the selected sequenced proximity ligated fragments.

11. The method according to claim 2, wherein the set of selection criteria for identifying the plurality of related genomic fragments that are related to the genomic fragment comprises at least one of:
a. whether a candidate related genomic fragment localizes in the reference genome in cis to the same chromosome that also harbors the genomic region of interest;
b. whether the candidate related genomic fragment localizes in the reference genome in cis to a specific part of the same chromosome that also harbors the genomic region of interest; and
c. whether the candidate related genomic fragment localizes in the reference genome in trans to a chromosome that does not harbor the genomic region of interest.

12. The method according to claim 2, wherein the set of selection criteria for identifying the plurality of related genomic fragments that are related to the genomic fragment comprises at least one of:
i. whether the candidate related genomic fragment localizes to a genomic part of a same active or inactive three-dimensional nuclear compartment (for example the A or B compartment) as the genomic region of interest, as determined by nuclear proximity assays.
ii. whether the candidate related genomic fragment localizes to a genomic part that has a same or a similar epigenetic chromatin profile as the genomic region of interest, as determined for example by an epigenetic profiling method that analyzes the genomic distribution of a given histone modification;
iii. whether the candidate related genomic fragment localizes to a genomic part that has a similar transcriptional activity as the genomic region of interest, as determined by a transcriptional profiling method;
iv. whether the candidate related genomic fragment localizes to a genomic part that has a similar replication timing as the genomic region of interest, as determined by a replication timing profiling method;
v. whether the candidate related genomic fragment localizes to a genomic part that has a related density of experimentally created fragments as the genomic region of interest; and
vi. whether the candidate related genomic fragment localizes to a genomic part that has a related density of non-mappable fragments or fragment ends as the genomic region of interest.

13. The method of claim 1, wherein the set of selection criteria for identifying the plurality of related genomic fragments comprises a requirement that the proximity score of the candidate related genomic fragment has a value indicative of a non-zero number of DNA reads, preferably wherein the generating the indication of the likelihood that said at least one genomic fragment is related to a chromosomal rearrangement comprises
generating a first indication of the likelihood that said at least one genomic fragment is related to a chromosomal rearrangement using a set of selection criteria excluding the requirement that the proximity score of the candidate related genomic fragment has a value indicative of a non-zero number of DNA reads;
generating a second indication of the likelihood that said at least one genomic fragment is related to a chromosomal rearrangement using the set of selection criteria including the requirement that the proximity score of the candidate related genomic fragment has a value indicative of a non-zero number of DNA reads; and
generating a third indication of the likelihood that said at least one genomic fragment is related to a chromosomal rearrangement, based on the first indication and the second indication.

14. A computer program product comprising computer-readable instructions that, when executed by a processor system, cause the processor system to:
assign an observed proximity score to each of a plurality of genomic fragments of a genome, the observed proximity score of a genomic fragment being indicative of a presence in a dataset of at least one DNA read corresponding to the genomic fragment, wherein the dataset comprises DNA reads, the DNA reads representing genomic fragments being in nuclear proximity to a genomic region of interest;
assign an expected proximity score to each of at least one genomic fragment of the plurality of genomic fragments, based on the observed proximity scores of the plurality of genomic fragments, wherein the expected proximity score is an expected value of the proximity score of the at least one of the plurality of genomic fragments; and
generate an indication of a likelihood that said at least one genomic fragment of the plurality of genomic fragments is involved in a chromosomal rearrangement, based on the observed proximity score of said at least one genomic fragment of the plurality of genomic fragments and the expected proximity score of said at least one genomic fragment of the plurality of genomic fragments.

15. (canceled)

16. A method for confirming the presence of a chromosomal breakpoint junction, fusing a candidate rearrangement partner to a position within a genomic region of interest, said method comprising:
a. performing a proximity assay on a DNA comprising sample to generate a plurality of proximity linked products;
b. enriching for proximity linked products that comprise genomic fragments comprising sequences flanking the 5′ end of the genomic region of interest,
wherein said proximity linked products further comprise genomic fragments being in proximity to said genomic fragments comprising sequences flanking the 5′ end of the genomic region of interest;
sequencing said proximity linked products to produce sequencing reads,
mapping to a reference sequence the sequences of the genomic fragments that are in proximity to said genomic fragments comprising sequences flanking the 5′ end of the genomic region of interest;
c. enriching for proximity linked products that comprise genomic fragments comprising sequences flanking the 3′ end of the genomic region of interest,
wherein said proximity linked products further comprise genomic fragments being in proximity to said genomic fragments comprising sequences flanking the 3′ end of the genomic region of interest;
sequencing said proximity linked products to produce sequencing reads,
mapping to a reference sequence the sequences of the genomic fragments that are in proximity to said genomic fragments comprising sequences flanking the 3′ end of the genomic region of interest;
d. identifying, as a candidate rearrangement partner, at least one genomic fragment based on the proximity frequency of said genomic fragment with the genomic region of interest or genomic fragments comprising sequences flanking the genomic region of interest,
e. determining whether genomic fragments of the candidate rearrangement partner that are in proximity to said genomic fragments comprising sequences flanking the 5′ end of the genomic region of interest and genomic fragments of the candidate rearrangement partner that are in proximity to said genomic fragments comprising sequences flanking the 3′ end of the genomic region of interest are overlapping or linearly separated,
wherein linear separation of said candidate rearrangement partner genomic fragments is indicative of a chromosomal breakpoint junction within the genomic region of interest.

17. The method of claim 16, wherein the proximity assay is a proximity ligation assay that generates a plurality of proximity ligated products.

18. The method of claim 16, wherein step b) comprises performing oligonucleotide probe hybridization or primer-based amplification to enrich for proximity linked products that comprise genomic fragments comprising sequences flanking the 5′ end of the genomic region of interest and/or step c) comprises performing oligonucleotide probe hybridization or primer-based amplification to enrich for proximity linked products that comprise genomic fragments comprising sequences flanking the 3′ end of the genomic region of interest, preferably
wherein step b) comprises providing at least one oligonucleotide probe or primer that is at least partly complementary to sequences flanking the 5′ region of the genomic region of interest, and/or
wherein step c) comprises providing at least one oligonucleotide probe or primer that is at least partly complementary to sequences flanking the 3′ region of the genomic region of interest.

19. The method of claim 16, further comprising determining the position of the chromosomal breakpoint junction fusing the candidate rearrangement partner to a position within the genomic region of interest, said method comprising:
enriching for proximity linked products that comprise i) at least part of the genomic region of interest and ii) genomic fragments being in proximity to the genomic region of interest sequencing said proximity linked products and mapping the chromosomal breakpoint, wherein the mapping comprises detecting I) proximity linked products comprising at least a first part of the genomic region of interest and genomic fragments of a rearrangement partner and II) proximity linked products comprising at least a second part of the genomic region of interest and genomic fragments of a rearrangement partner, wherein the rearrangement partner genomic fragments from I) and II) are linearly separated, preferably comprising performing oligonucleotide probe hybridization or primer-based amplification to enrich for proximity linked products that comprise i) at least part of the genomic region of interest and ii) genomic fragments being in proximity to the genomic region of interest.

20. The method of claim 16, comprising generating a matrix for at least a subset of the sequencing reads, wherein one axis of the matrix represents the sequence location of the genomic region of interest and/or the region flanking the genomic region of interest and the other axis represent the sequence location of the candidate rearrangement partner, wherein the matrix is generated by superimposing the sequencing reads over the matrix such that each element within the matrix represents the frequency of a proximity linked product identified that comprises a genomic fragment of the genomic region of interest or flanking the region of interest and a genomic fragment from the rearrangement partner, preferably wherein the matrix is a butterfly plot.

21. The method of claim 16, further comprising determining the sequence of a genomic region spanning the breakpoint, said method comprising
identifying proximity linked products comprising i) breakpoint-proximal genomic fragments of the genomic region of interest and ii) rearrangement partner genomic fragments.

22. The method of claim 16, wherein step d) comprises
assigning an observed proximity score to each of a plurality of genomic fragments of a genome, the observed proximity score of each genomic fragment being indicative of a presence in the dataset of at least one sequencing read in proximity to the genomic region of interest and comprising a sequence corresponding to the genomic fragment;
assigning an expected proximity score to each of at least one genomic fragment of the plurality of genomic fragments, based on the observed proximity scores of the plurality of genomic fragments, wherein the expected proximity score comprises an expected value of the proximity score of the at least one of the plurality of genomic fragments; and
generating an indication of a likelihood that said at least one genomic fragment of the plurality of genomic fragments is involved in a chromosomal rearrangement, based on the observed proximity score of said at least one genomic fragment of the plurality of genomic fragments and the expected proximity score of said at least one genomic fragment of the plurality of genomic fragments and identifying said genomic fragment as a candidate rearrangement partner.

23. A method for confirming the presence of a chromosomal breakpoint junction, fusing a candidate rearrangement partner to a position within a genomic region of interest, said method comprising:
defining a genomic region of interest;
performing a proximity assay on a DNA comprising sample to generate a plurality of proximity linked products;
enriching for proximity linked products that comprise genomic fragments comprising sequences flanking the 5′ end of the genomic region of interest,
wherein said proximity linked products further comprise genomic fragments being in proximity to said genomic fragments comprising sequences flanking the 5′ end of the genomic region of interest;
sequencing said proximity linked products to produce sequencing reads,
mapping to a reference sequence the sequences of the genomic fragments that are in proximity to said genomic fragments comprising sequences flanking the 5′ end of the genomic region of interest;
enriching for proximity linked products that comprise genomic fragments comprising sequences flanking the 3′ end of the genomic region of interest,
wherein said proximity linked products further comprise genomic fragments being in proximity to said genomic fragments comprising sequences flanking the 3′ end of the genomic region of interest;
sequencing said proximity linked products to produce sequencing reads,
mapping to a reference sequence the sequences of the genomic fragments that are in proximity to said genomic fragments comprising sequences flanking the 3′ end of the genomic region of interest;
enriching for proximity linked products that comprise i) at least part of the genomic region of interest and ii) genomic fragments being in proximity to the genomic region of interest;
sequencing said proximity linked products to produce sequencing reads,
mapping to a reference sequence the sequences of the genomic fragments that are in proximity to the genomic region of interest;
identifying, as a candidate rearrangement partner, at least one genomic fragment based on the proximity frequency of said genomic fragment with the genomic region of interest or genomic fragments comprising sequences flanking the genomic region of interest, preferably by assigning an observed proximity score to each of a plurality of genomic fragments of a genome, the observed proximity score of each genomic fragment being indicative of a presence in the dataset of at least one sequencing read in proximity to the genomic region of interest and comprising a sequence corresponding to the genomic fragment;
assigning an expected proximity score to each of at least one genomic fragment of the plurality of genomic fragments, based on the observed proximity scores of the plurality of genomic fragments, wherein the expected proximity score comprises an expected value of the proximity score of the at least one of the plurality of genomic fragments; and
generating an indication of a likelihood that said at least one genomic fragment of the plurality of genomic fragments is involved in a chromosomal rearrangement, based on the observed proximity score of said at least one genomic fragment of the plurality of genomic fragments and the expected proximity score of said at least one genomic fragment of the plurality of genomic fragments and identifying said genomic fragment as a candidate rearrangement partner;
determining whether genomic fragments of the candidate rearrangement partner that are in proximity to said genomic fragments comprising sequences flanking the 5′ end of the genomic region of interest and genomic fragments of the candidate rearrangement partner that are in proximity to said genomic fragments comprising sequences flanking the 3′ end of the genomic region of interest are overlapping or linearly separated,
wherein linear separation of said candidate rearrangement partner genomic fragments is indicative of a chromosomal breakpoint junction within the genomic region of interest;
mapping the location of the chromosomal breakpoint, comprising detecting I) proximity linked products comprising at least a first part of the genomic region of interest and genomic fragments of a rearrangement partner and II) proximity linked products comprising at least a second part of the genomic region of interest and genomic fragments of a rearrangement partner, wherein the rearrangement partner genomic fragments from I) and II) are linearly separated.

24. A computer program product for detecting a chromosomal breakpoint fusing a rearrangement partner to a position within a genomic region of interest, said computer program product comprising computer-readable instructions that, when executed by a processor system, cause the processor system to:
generate a matrix for at least a subset of sequencing reads, wherein the sequencing reads correspond to the sequences of proximity linked products, said products comprising genomic fragments from the genomic region of interest or flanking the region of interest and wherein at least a subset of proximity linked products comprises a genomic fragment of a candidate rearrangement partner,
wherein one axis of the matrix represents the sequence location of the genomic region of interest and/or region flanking the genomic region of interest and the other axis represent the sequence location of the candidate rearrangement partner, wherein the matrix is generated by superimposing the sequencing reads over the matrix such that each element within the matrix represents the frequency of a proximity linked product that comprises a genomic segment of the genomic region of interest or flanking the region of interest and a genomic segment from the rearrangement partner, and
search the matrix to detect one or more coordinates on the axis representing the sequence location of the genomic region of interest and/or region flanking the genomic region of interest that shows a transition in proximity frequency of the genomic segments from the candidate rearrangement partner.

25. The computer program product of claim 24, wherein the processor system searches the matrix to detect one or more coordinates on the axis representing the sequence location of the genomic region of interest and/or region flanking the genomic region of interest that divides at least a part of the matrix into four quadrants, such that the differences in frequency between adjacent quadrants is maximized and the differences between opposing quadrants is minimized, preferably wherein the processor system
compares the four quadrants identified and
classifies the chromosomal breakpoint as resulting in a reciprocal rearrangement when two opposing quadrants exhibit minimal difference in frequency and the adjacent quadrants exhibit maximal differences in frequency or classifies the chromosomal breakpoint as resulting in a non-reciprocal rearrangement when a single quadrant exhibits the maximal difference in frequency compared to the other three quadrants.

26. The method according to claim 16 any one of claims 15-23 comprising detecting a chromosomal breakpoint fusing a rearrangement partner to a position within a genomic region of interest using the computer program product of claim 24 any one of claims 24-25.
